(19) United States
(12) Patent Application Publication
(10) Pub. No.: US 2008/0269754 A1
(43) Pub. Date: Oct. 30, 2008

(54) Preparation Tools and Methods of Using the Same

(75) Inventors: Gregory E. Lutz, Princeton, NJ (US); Hans Hull, Princeton, NJ (US); Jimmy Lin, Somerville, NJ (US)

Correspondence Address: EDELL, SHAPIRO & FINNAN, LLC, 1901 RESEARCH BOULEVARD, SUITE 400, ROCKVILLE, MD, 20850, US

(73) Assignee: Orthobond, Inc., Monmouth Junction, NJ (US)

(21) Appl. No.: 12/042,979

(22) Filed: Mar. 5, 2008

Related U.S. Application Data
(60) Provisional application No. 60/893,355, filed on Mar. 6, 2007.
(60) Provisional application No. 60/910,228, filed on Apr. 5, 2007.
(60) Provisional application No. 60/977,639, filed on Oct. 4, 2007.
(60) Provisional application No. 61/021,609, filed on Jan. 16, 2008.

(51) Int. Cl. A61B 17/00; A61B 17/32

(52) U.S. Cl. 606/79; 606/167; 606/170

(57) ABSTRACT

Various devices and methods for accessing and preparing treatment sites within the intervertebral disc space for subsequent negligible-incision surgical (NIS) or percutaneous procedures to treat disc degeneration and disc related back pain are disclosed. Also disclosed is a method for performing a percutaneous spine procedure including preparing a treatment site within the intervertebral disc space for subsequent delivery of a biomaterial to treat disc degeneration and disc related back pain.

### REFERENCE TO RELATED PATENT APPLICATIONS

This patent application claims the benefit of U.S. Provisional Application No. 60/893,355, filed Mar. 6, 2007, entitled “Preparation Tools and Methods of Using the Same,” having Attorney Docket No. 1526.0004P, and the benefit of U.S. Provisional Application No. 60/910,228, filed Apr. 5, 2007, entitled “A Method For a Percutaneous Spine Procedure,” having Attorney Docket No. 2917.0002P, and the benefit of U.S. Provisional Application No. 60/977,639, filed Oct. 4, 2007, entitled “Preparation Tools and Methods of Using the Same,” having Attorney Docket No. 1526.0005P, and the benefit of U.S. Provisional Application No. 61/021,609, filed Jan. 16, 2008, entitled “Preparation Tools and Methods of Using the Same,” having Attorney Docket No. 1526.0006P, the disclosures of each of which is incorporated by reference herein in its entirety.

### FIELD OF THE INVENTION

The present invention relates generally to instrumentation systems and methods for accessing and preparing treatment sites within the intervertebral disc space or spinal facet joint for subsequent negligible-incision surgical (NIS) or percutaneous procedures to treat disc degeneration, disc related back pain and facet joint osteoarthritis, such as, for example, arthrodesis, discectomy, nucleotomy, annular repair or the like.

### BACKGROUND OF THE INVENTION

The human spine or spinal column 12 in a human body 10 (see FIG. 1A) is a segmented, semi-constrained, weight bearing musculo-skeletal structure capable of simultaneous flexion and rotation. The spine 12 is a stacked series of motion segments or vertebrae 14. The vertebrae 14 in the spine 12 are often classified into four sections: cervical, thoracic, lumbar and sacral. The cervical spine comprises the seven vertebral segments 20 of the neck. The thoracic spine has the twelve vertebrae 22 below the cervical spine. Below the thoracic spine are the five lumber vertebrae 24 and then the five sacral vertebrae. The sacral vertebrae are fused into a structure called the sacrum 16. The coccyx 18 is also illustrated. The motion segments of the spine are held together and surrounded by ligaments, strong fibrous soft-tissues that firmly attach bones to bones.

The major structural components of each spinal motion segment, shown in (FIGS. 1A-1C), are the vertebral body 28, the posterior structures and facet joints and the intervertebral disc 36. The vertebral body 28 is oval cylindrical segment of bone with an outer layer of dense cortical bone 30 and an interior of spongey, vascularized cancellous bone 34. The superior and inferior surfaces 50 and 52 of the vertebral body 28 where the intervertebral disc 36 connects to the vertebral body 28 are called “end plates.” Under normal conditions, the endplate is a layered, concave surface made up of a layer of cartilage on top of a layer of cortical bone 30. The end plate cortical bone layer is thickest towards the perimeter of the vertebral body and progressively thins towards its center. The surface of end plates are vascularized and innervated. The posterior vertebral structures form the vertebral canal which protects the spinal cord. These structures include the facet joints, lamina, pedicles, spinous process, transverse process, superior and inferior articular processes, and the mammilary processes.

Referring to FIGS. 1B and 1C, an opening, called the vertebral foramen 38, is located on the posterior (i.e., back) side of each vertebra 14. The spinal ganglion 41 passes through the foramen 38. The spinal cord 40 passes through the spinal canal 39. The vertebral arch surrounds the spinal canal 39. The pedicle 44 of the vertebral arch 42 adjoins the vertebral body 28. The spinous process 46 extends from the posterior of the vertebral arch, as do the left and right transverse processes 48.

As illustrated in FIGS. 1C, 2A, and 2B, the intervertebral disc 36 lies in the space below the inferior end plate 50 of one vertebrae 28 and above the superior end plate 52 of the next vertebrae 28. This space is called the “intervertebral disc space” or “disc space.” In FIGS. 2A and 2B, the anterior (A) and posterior (P) orientations of the functional spine unit are illustrated. Also shown are an intervertebral disc 36, the left 58 and right 60 transverse processes, and the spinous process 62. The intervertebral disc 36 is a pad of fibrocartilage made up of two concentric structures, the annulus fibrosus 54 and the nucleus pulposus 56. The annulus fibrosis 54, a series of concentric rings of fibrocartilage tissue called lamellae, forms the perimeter of the disc and outer layers of the disc 36. The annulus 54 surrounds the nucleus populous 56, a proteoglycan and water gel held together loosely by an irregular network of fine collagen type II and elastin fibers. A young healthy disc 36 behaves like a water bed, with the high water content of the nucleus 56 and inner annulus 54 enabling the tissue to act like a fluid and distribute mechanical and rotational load.

With age, intervertebral discs undergo a process called disc degeneration resulting in structural and biochemical changes to the disc and vertebral end plates, often resulting in disc related pain. Injury and genetic factors contribute to the degenerative process. As they degenerate, discs lose fluid and stiffen. Additionally, the nucleus 56 progressively dehydrates becoming less fluid, more viscous and less able to effectively distribute load. The annulus 54 tends to thicken, desiccate and become more rigid, reducing its ability to elastically deform under and distribute mechanical load. These changes increase the susceptibility of the annulus 54 to fracture and fissures and the likelihood of disc herniation. Changes to the end plates include sclerosis, calcification, formation of osteophytes, nerve inflammation and deformation of the end plate surface which tends to flatten.

FIG. 2B is a sectional view through the midline of two adjacent vertebral bodies 70 (superior) and 72 (inferior). Intervertebral disc space 75 is formed between the two vertebral bodies 70 and 72 and contains intervertebral disc 36, which supports and cushions the vertebral bodies 70 and 72 and permits movement of the two vertebral bodies 70 and 72 with respect to each other and other adjacent functional spine units.

Intervertebral disc 36 is comprised of the annulus 54 which normally surrounds and constrains the nucleus 56 to be wholly within the borders of the intervertebral disc space 75. The vertebrae also include facet joints 74 and the superior 76 and inferior 77 pedicle that form the neural foramen 78.

As illustrated, vertebral body 70 includes an inferior endplate 50 that defines a portion of the disc space 75. Similarly, vertebral body 72 includes a superior endplate 52 that defines another portion of the disc space 75. The endplates 50 and 52 function in part to maintain the cancellous bone material 71 and 73 within the bodies 70 and 72, respectively.

Chronic back pain from degenerative disc disease (DDD) is a common cause of disability that results in decreased productivity, lost work time and significant health care costs. Treatments for DDD range from conservative care, e.g. heat, rest, pain relief medications, rehabilitation exercises and anti-inflammatory epidural injections, to more invasive surgical treatments such as nucleus removal (nucleotomy), disc removal (discectomy), various spinal arthroplasties, vertebral fusion (spinal arthrodesis) and implantation of so called motion preserving or dynamic stabilization implants.

Despite the array of treatments, outcomes are often unsatisfactory because therapeutic procedures may not lead to pain relief. This may be due in part to the multiple sources of DDD related pain which can be caused by one or more of the following: bulging of the annulus or PLL with subsequent nerve impingement; tears, fissures or cracks in the outer, innervated layers of the annulus; motion induced leakage of nuclear material through the annulus and subsequent irritation of surrounding tissue in response to the foreign body reaction, facet pain, end plate inflammation pain.

Sufferers of DDD who have failed conservative treatment have few choices other than to live with their pain or undergo surgery. Surgical treatment has significant drawbacks including damage to healthy spinal anatomy, blood loss, risk of complications such as infection, lengthy recovery times and increased adjacent segment disease progression. Increasingly, efforts have been made to develop minimally invasive surgical treatments for DDD to minimize the drawbacks of surgery. Minimally invasive surgery, in contrast to conventional or open surgery, involves insertion of a surgical device through a smaller incision, often using a tube or cannula.

Despite these advances, the incisions required for minimally invasive surgical treatments of DDD still require cutting and/or removal of healthy anatomy to access the disc space. These structures, including the lamina, spinal ligaments, muscles and fascia contribute to spinal stability and function. There remains a need for tools and methods to treat degenerative disc disease, disc related pain, facet pain and facet osteoarthritis that conserve anatomy and do not require incisions, but allow access to, preparation of and delivery of treatment to the site of pathology and/or source of pain.

### BRIEF SUMMARY OF THE INVENTION

The present invention provides tools and methods for negligible-incision surgical (NIS) treatment of the intervertebral disc, degenerative disc disease (DDD), and associated pathologies including disc related pain as well as osteoarthritis and facet pain. As used herein, the term “negligible-incision surgery” is defined as the treatment of diseases and conditions by manual or operative procedures with tools of sufficiently small size that they may directly inserted into anatomy without a separate or prior incision of the muscle, tendons or ligaments. A small or “negligible” incision of the skin or dermis may facilitate the insertion of tools into the body and is within the meaning the term “negligible-incision.” NIS treatment of DDD requires tools that are small enough to be inserted into the intervertebral disc space through a percutaneous cannula or needle or that can be directly inserted into the disc space. NIS treatment of facet joints requires tools that are small enough to be inserted between the superior and inferior articulating surfaces of the facet joint. The term “negligible-incision surgical manner” relates to negligible-incision surgery.

The many benefits of negligible-incision surgery include minimal blood loss, tissue and muscle trauma, preservation of the anatomical structure of the spine, reduced neurological and infection risk, reduced procedure time and hospitalization period, pain reduction and increased functionality. NIS tools may be used to treat DDD via a variety of surgical approaches including postero-lateral, anterior, and trans-lateral. When used with a posterolaterial vertebral approach, the tool may be sized to fit a 10 gauge (outer diameter (“OD”) of 3.4 millimeters), 12 gauge (outer diameter (“OD”) of 2.769 millimeters), 14 gauge (OD of 2.108 millimeters), 16 gauge (OD of 1.651 millimeters), 18 gauge (OD of 1.27 millimeters) or smaller needle. NIS tools may be used to treat the facets via a variety of surgical approaches including the posterior and postero-lateral approaches. Because of the intrafacet joint space is generally smaller than the intravertebral disc space, the tool may be sized to fit a 16 gauge (OD of 1.651 millimeters), 18 gauge (OD of 1.27 millimeters), 20 gauge (OD of 0.95 millimeters), 22 gauge (OD of 0.7 millimeters) or smaller needle. The tools can be sized to fit other sized needles between a 10 gauge needle and 22 gauge needle. The NIS tools of the present invention may be used by surgeons and other qualified interventional medical professionals in an operating room or other appropriate setting to perform NIS procedures.

In contrast to minimally invasive surgical (MIS) tools for treatment of the spine, e.g. the METRx™ and TANGENT™ surgical systems available from Medtronic, the NIS tools of the present invention enable direct access to the intervertebral disc space without a surgical incision of the fascia or muscles and with preservation of the anatomical structure of the spine. The NIS tools disclosed as part of the present invention may be used to treat advanced stages of disc degeneration, e.g. degenerated discs of grades III, IV and V. The intervertebral space typically loses height at advanced stages of disc degeneration increasing the difficulty of accessing the disc space with surgical tools without distraction of the end plates and associated trauma to the surrounding tissue. At times, loss of disc height due to DDD allows the superior and inferior end plates to come into contact causing inflammation and pain.

Advantageously, the NIS tools disclosed as part of the present invention allow access to and treatment of the disc space and end plates even in advanced cases of disc degeneration in which the disc has lost significant height. In certain embodiments, the tools of the present invention allow the physician to feel the anatomy in and around the disc space enabling the physician to judge the extent of the disease and nature of the treatment required.

The present invention also comprises methods of use of the disclosed tools to promote and/or facilitate NIS fusion of adjacent vertebrae or facet joint. Vertebral arthrodesis or fusion is a common treatment for DDD. This method may be used to fuse vertebrae or facet joints in the cervical, thoracic, lumbar and sacralilliac spine. According to one method of the present invention, a physician seeking to fuse two adjacent vertebrae of a patient via NIS, percutaneously creates a pathway to the perimeter of the disc space or the facet joint. Said pathway is initiated via insertion of a needle or cannula rather than via an incision. The needle or catheter may be inserted under imaging or tactile guidance. Examples of such imaging guidance include radiographic guidance such as with a fluoroscope, CT scan, X-Ray, or MRI, visual guidance such as with an endoscope, laparoscope, fiber optic or other camera. Insertion under tactile guidance would be via contact with known anatomy during insertion.

Following creation of a pathway to the disc space or facet joint, a tool of the present invention is inserted into the disc space or facet joint. After insertion, the tool is manipulated by the physician, either manually, via hand actuation or with a powered actuating means to engage the disc material and the superior and/or inferior end plate or the superior and/or inferior facet joint articulating surface. The tool may be manipulated to disrupt the disc material, disrupt or remove the fibrocartilage layer of the end plates and/or facet joint articulating surfaces and create a roughened and bleeding surface on the end plates and/or facet joint articulating surfaces. The tool, if steerable, may be manipulated to maximize the surface area of the end plates engaged by the tool.

The disrupted disc material and debris from the end plates and/or facet joints may optionally be removed via standard irrigation and aspiration techniques known to one of skill in the art. Also optionally, osteoconductive, osteoinductive materials and carrier materials or both may be injected into the disc space along the previously created percutaneous pathway. Following preparation of the disc space, the tool and then the insertion device are removed.

In an alternative embodiment of the method of use of the disclosed tools to promote or facilitate NIS fusion of adjacent vertebrae or facet surfaces, the tool is inserted across the disc space under guidance. Upon reaching the point of maximum safe insertion, the physician applies a clip, stop or other device known to one of skill in the art, to the shaft of the tool to prevent its insertion beyond the maximum safe depth.

In an alternative embodiment of the method of use of the disclosed tools to promote or facilitate NIS fusion of adjacent vertebrae or fusion of a facet joint, whole or concentrated autologous or allograft materials, either alone or in combination with other agents may be injected at the treatment site along the previously created percutaneous pathway.

The present invention provides in another aspect, a method of performing a percutaneous spine procedure on a patient. The method includes inserting a delivery device into a spinal column of the patient. The method includes further establishing a percutaneous pathway using the delivery device that leads from a skin exit location to a disc space defined by at least one vertebral endplate or to a facet joint space defined by at least one facet articulating surface. The method also provides for introducing a preparation device through the delivery device into the disc space or facet joint. The preparation device has a support portion and a cutting portion with the cutting portion of the preparation device being selectively disposable in a delivery configuration and in a deployed configuration relative to the support portion. Further, the method includes preparing the disc space or facet joint by engaging the cutting portion of the preparation device with at least one vertebral endplate. The method also includes delivering a biomaterial or autologous material through the delivery device to the prepared disc space or facet joint to facilitate forming at least a partial arthrodesis between two adjacent endplates. Further, additional features and advantages are realized through the techniques of the present invention. Other embodiments and aspects of invention are described in detail herein and are considered a part of the claimed invention.

### BRIEF DESCRIPTION OF THE DRAWINGS

The subject matter which is regarded as the invention is particularly pointed out and distinctly claimed in the claims at the conclusion of the specification. The foregoing and other objects, features, and advantages of the invention are apparent from the following detailed description taken in conjunction with the accompanying drawings in which:

FIG. 1A is a side view of a spinal column of a human, in accordance with an aspect of the present invention;

FIG. 1B is coronal view of a lumbar vertebra, partially cut away and in section, taken along line “1B-1B” in FIG. 1A, in accordance with an aspect of the present invention;

FIG. 1C is a vertical section view of lumbar vertebrae, in accordance with an aspect of the present invention;

FIGS. 2A and 2B are plan and partial cross-sectional side views of an exemplary disc space, in accordance with an aspect of the present invention;

FIGS. 3 and 4 are schematic diagrams illustrating an exemplary use of a tool according to the invention, in accordance with an aspect of the present invention;

FIG. 5A is a block diagram showing some of the components of an embodiment of a tool according to the invention, in accordance with an aspect of the present invention;

FIG. 5B is a block diagram showing some of the components of another embodiment of a tool according to the invention, in accordance with an aspect of the present invention;

FIGS. 6A-6C are various embodiments of controlling portions for a tool according to the invention, in accordance with an aspect of the present invention;

FIG. 7 is a side view of an embodiment of a tool according to the invention, in accordance with an aspect of the present invention;

FIG. 8 is a side view of the tool of FIG. 7 and an embodiment of a delivery device, in accordance with an aspect of the present invention;

FIG. 9 is a schematic view of another embodiment of a tool according to the invention, in accordance with an aspect of the present invention;

FIG. 10-11 are views of another embodiment of a tool according to the invention, in accordance with an aspect of the present invention;

FIGS. 12-14 are side views of another embodiment of a tool according to the invention, in accordance with an aspect of the present invention;

FIGS. 15-16 are a side view and an end view, respectively, of the tool illustrated in FIGS. 12-14 in a deployed configuration, in accordance with an aspect of the present invention;

FIGS. 17-21 are side views and top views of another embodiment of a tool according to the invention, in accordance with an aspect of the present invention;

FIGS. 22-23 are a side view and an end view, respectively, of the tool illustrated in FIGS. 17-21 in a deployed configuration, in accordance with an aspect of the present invention;

FIGS. 24-25 are side views of another embodiment of a tool according to the invention, in accordance with an aspect of the present invention;

FIGS. 26-28 are a side view, an end view, and a bottom view, respectively, of the tool of FIGS. 24-25 in a deployed configuration, in accordance with an aspect of the present invention;

FIGS. 29-30 are side views of another embodiment of a tool according to the invention, in accordance with an aspect of the present invention;

FIGS. 31-33 are a side view, an end view, and a bottom view, respectively, of the tool of FIGS. 29-31 in a deployed configuration, in accordance with an aspect of the present invention;

FIGS. 34-35 are a side view and a top view, respectively, of another embodiment of a tool according to the invention, in accordance with an aspect of the present invention;

FIG. 36 is a side view of the tool of FIGS. 34-35 in a deployed configuration, in accordance with an aspect of the present invention;

FIGS. 37-38 are a side view and a top view, respectively, of another embodiment of a tool according to the invention, in accordance with an aspect of the present invention;

FIG. 39 is a side view of another embodiment of a tool according to the invention, in accordance with an aspect of the present invention;

FIG. 40 is a side view of the tool of FIG. 39 in a deployed configuration, in accordance with an aspect of the present invention;

FIG. 41 is a close-up side view of a portion of the tool of FIG. 40, in accordance with an aspect of the present invention;

FIG. 42 is a side view of another embodiment of a tool according to the invention, in accordance with an aspect of the present invention;

FIGS. 43 and 44 are a side view and an end view, respectively, of the tool of FIG. 42 in a deployed configuration, in accordance with an aspect of the present invention;

FIG. 45 is a side view of another embodiment of a tool according to the invention, in accordance with an aspect of the present invention;

FIGS. 46 and 47 are a side view and an end view, respectively, of the tool of FIG. 45 in a deployed configuration, in accordance with an aspect of the present invention;

FIGS. 48-49 are a top view and a side view of another embodiment of a tool according to the invention, in accordance with an aspect of the present invention;

FIGS. 50-51 are a side view and an end view of the tool of FIGS. 48-49, in accordance with an aspect of the present invention;

FIG. 52 is a perspective view of another embodiment of a tool according to the invention, in accordance with an aspect of the present invention;

FIG. 53 is a close-up perspective view of a portion of the tool of FIG. 52, in accordance with an aspect of the present invention;

FIG. 54 is a cross-sectional end view taken along the lines “54“−”54” in FIG. 53, in accordance with an aspect of the present invention;

FIG. 55 is a side view of a portion of the tool of FIG. 52, in accordance with an aspect of the present invention;

FIG. 56 is a perspective view of another embodiment of a tool according to the invention, in accordance with an aspect of the present invention;

FIG. 57 is a close-up perspective view of a portion of the tool of FIG. 56, in accordance with an aspect of the present invention;

FIG. 58 is a side view of a portion of the tool of FIG. 56, in accordance with an aspect of the present invention;

FIG. 59 is a partial cross-sectional side view of another embodiment of a tool according to the invention in a delivery configuration, in accordance with an aspect of the present invention;

FIG. 60 is a partial cross-sectional side view of the tool of FIG. 59 in a deployed configuration, in accordance with an aspect of the present invention;

FIGS. 61-63 are a perspective view, a front view, and a side view, respectively, of an exemplary cutting element of the tool of FIG. 59, in accordance with an aspect of the present invention;

FIG. 64 is a partial cross-sectional side view of another embodiment of a tool according to the invention in a delivery configuration, in accordance with an aspect of the present invention;

FIG. 65 is a partial cross-sectional side view of the tool of FIG. 64 in a deployed configuration, in accordance with an aspect of the present invention;

FIG. 66 is a perspective view of an exemplary cutting element of the tool of FIG. 64, in accordance with an aspect of the present invention;

FIG. 67 is an end view of an arrangement of some cutting elements of the tool of FIG. 64 in a deployed configuration, in accordance with an aspect of the present invention;

FIG. 68 is an end view of an alternative arrangement of some cutting elements of the tool of FIG. 64 in a deployed configuration, in accordance with an aspect of the present invention;

FIG. 69 is a partial cross-section view of another embodiment of a tool according to the invention in a delivery configuration, in accordance with an aspect of the present invention;

FIG. 70 is a partial cross-section view of the tool of FIG. 69 illustrated in a deployed configuration, in accordance with an aspect of the present invention;

FIG. 71 is a view of a rod of the tool of FIG. 69, in accordance with an aspect of the present invention;

FIG. 72 is a perspective view of an exemplary embodiment of a cutting element of the tool of FIG. 69, in accordance with an aspect of the present invention;

FIG. 73-74 are views of another embodiment of a tool according to the invention in different configurations, in accordance with an aspect of the present invention;

FIG. 75-77 illustrate additional embodiments of a tool according to the invention, in accordance with an aspect of the present invention;

FIG. 78 is a perspective view of an embodiment of a delivery device, in accordance with an aspect of the present invention;

FIG. 79 is an end view of the delivery device illustrated in FIG. 78, in accordance with an aspect of the present invention;

FIG. 80 is a partial cross-sectional side view of an embodiment of a site preparation tool in a delivery configuration, in accordance with an aspect of the present invention;

FIG. 81 is a side view of the site preparation tool illustrated in FIG. 80 in a deployed configuration, in accordance with an aspect of the present invention;

FIG. 82 is a side view of the site preparation tool illustrated in FIG. 80 in another deployed configuration.

FIG. 83 is a side view of an embodiment of a preparation device in a delivery configuration, in accordance with an aspect of the present invention;

FIG. 84 is a side view of an embodiment of the preparation device illustrated in FIG. 83 in a deployed configuration, in accordance with an aspect of the present invention;

FIG. 85 is an exploded perspective view of the preparation device illustrated in FIG. 83, in accordance with an aspect of the present invention;

FIG. 86 is an end view of an embodiment of cutting elements in a delivery configuration, in accordance with an aspect of the present invention;

FIG. 87 is an end view of the cutting elements illustrated in FIG. 86 in a deployed configuration, in accordance with an aspect of the present invention;

FIG. 88 is a cross-sectional end view of the preparation device illustrated in FIG. 83 taken along the line “88-88”, in accordance with an aspect of the present invention;

FIG. 89 is an end view of another embodiment of cutting elements, in accordance with an aspect of the present invention;

FIG. 90 is an end view of another embodiment of a site preparation tool, in accordance with an aspect of the present invention;

FIG. 91 is an end view of another embodiment of a site preparation tool, in accordance with an aspect of the present invention;

FIG. 92 is an end view of another embodiment of a site preparation tool, in accordance with an aspect of the present invention;

FIG. 93 is a perspective view of an embodiment of an actuating component, in accordance with an aspect of the present invention;

FIG. 94 is a side view of the actuating component illustrated in FIG. 93, in accordance with an aspect of the present invention;

FIG. 95 is a side view of another embodiment of an actuating component, in accordance with an aspect of the present invention;

FIG. 96 is a side view of another embodiment of an actuating component, in accordance with an aspect of the present invention;

FIG. 97 is a side view of another embodiment of an actuating component, in accordance with an aspect of the present invention;

FIG. 98 is a side schematic view of another embodiment of a site preparation tool, in accordance with an aspect of the present invention;

FIG. 99 is a side view of another embodiment of a site preparation tool in a delivery configuration, in accordance with an aspect of the present invention;

FIG. 100 is a side view of the site preparation tool illustrated in FIG. 99 in a deployed configuration, in accordance with an aspect of the present invention;

FIG. 101 is a perspective view of the cutting tool of the site preparation tool illustrated in FIG. 99 in a delivery configuration, in accordance with an aspect of the present invention;

FIG. 102 is a perspective view of the cutting tool illustrated in FIG. 101 in a deployed configuration, in accordance with an aspect of the present invention;

FIG. 103 is an end view of the cutting tool illustrated in FIG. 101, in accordance with an aspect of the present invention;

FIG. 104 is a side view of the actuator of the site preparation tool illustrated in FIG. 99, in accordance with an aspect of the present invention;

FIGS. 105A and 105B are side views of an embodiment of a control mechanism showing some of the internal components, in accordance with an aspect of the present invention;

FIG. 106 is a close-up side view of the control mechanism illustrated in FIGS. 105A and 105B, in accordance with an aspect of the present invention;

FIG. 107 is a perspective view of the housing of the control mechanism illustrated in FIGS. 105 and 105B, in accordance with an aspect of the present invention;

FIG. 108 is an end view of an embodiment of an actuator of the control mechanism illustrated in FIGS. 105 and 105B, in accordance with an aspect of the present invention;

FIG. 109 is a perspective view of an embodiment of a controller of the control mechanism illustrated in FIGS. 105 and 105B, in accordance with an aspect of the present invention;

FIG. 110 is a side view of another embodiment of a site preparation tool in a delivery configuration, in accordance with an aspect of the present invention;

FIG. 111 is a side view of the cutting tool of the site preparation tool illustrated in FIG. 110, in accordance with an aspect of the present invention;

FIG. 112 is an end view of the cutting tool illustrated in FIG. 111, in accordance with an aspect of the present invention;

FIG. 113 is a side view of an expander portion of the site preparation tool illustrated in FIG. 110, in accordance with an aspect of the present invention;

FIG. 114 is an end view of the expander portion illustrated in FIG. 113, in accordance with an aspect of the present invention;

FIG. 115 is a side view of another expander portion of the site preparation tool illustrated in FIG. 110, in accordance with an aspect of the present invention;

FIG. 116 is an end view of the expander portion illustrated in FIG. 115, in accordance with an aspect of the present invention;

FIG. 117 is a side view of the cutting tool illustrated in FIG. 110 in a deployed configuration, in accordance with an aspect of the present invention; and

FIG. 118 is a schematic diagram illustrating the delivery of biomaterial into the disc space using the tool, in accordance with an aspect of the present invention.

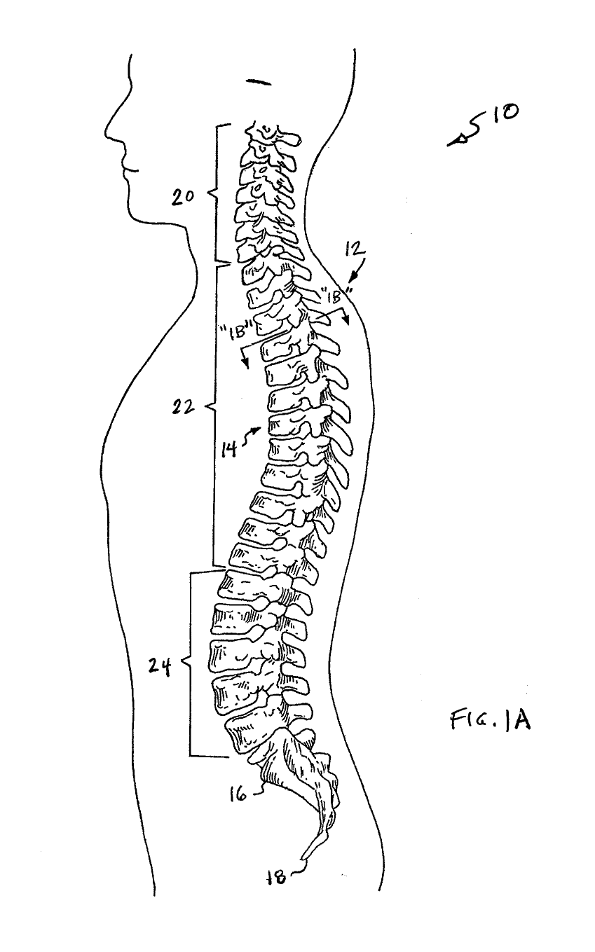

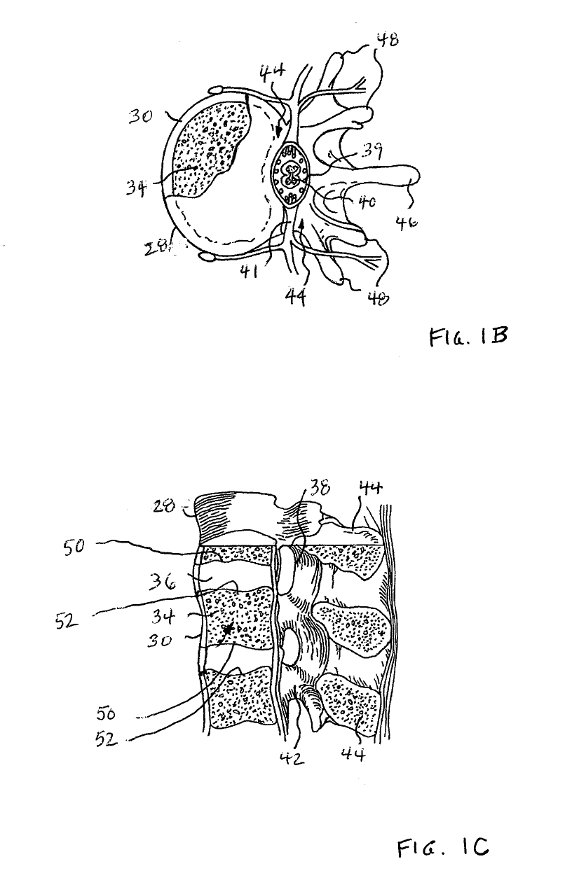

### DETAILED DESCRIPTION OF THE INVENTION

As described above, the present invention relates to tools and methods for NIS treatment of the intervertebral disc, DDD, and associated pathologies including disc related pain. In one embodiment, a tool is inserted to into the disc space and manipulated to engage the disc material and the superior and/or inferior end plate. The tool may be manipulated to disrupt the disc material, disrupt or remove the fibrocartilage layer of the end plates and create a roughened and bleeding surface on the end plates. The tool, if steerable, may be manipulated to maximize the surface area of the end plates engaged by the tool.

Referring to FIGS. 3 and 4, an exemplary method of using a tool according to the invention is illustrated. Tool 100 includes a delivery device 102 and a preparation or engaging device 104. The delivery device 102 can be a needle, cannula or other tube-like structure that has an internal channel through which the preparation or engaging device 104 can be inserted. The delivery device 102, as well as the other delivery devices described herein, has an outer diameter dimension (see “OD” in FIG. 3). The delivery device 102 is inserted into the patient's body and moved inwardly until its distal end 103 is located within the disc space 110 as defined in part by endplates 106 and 108.

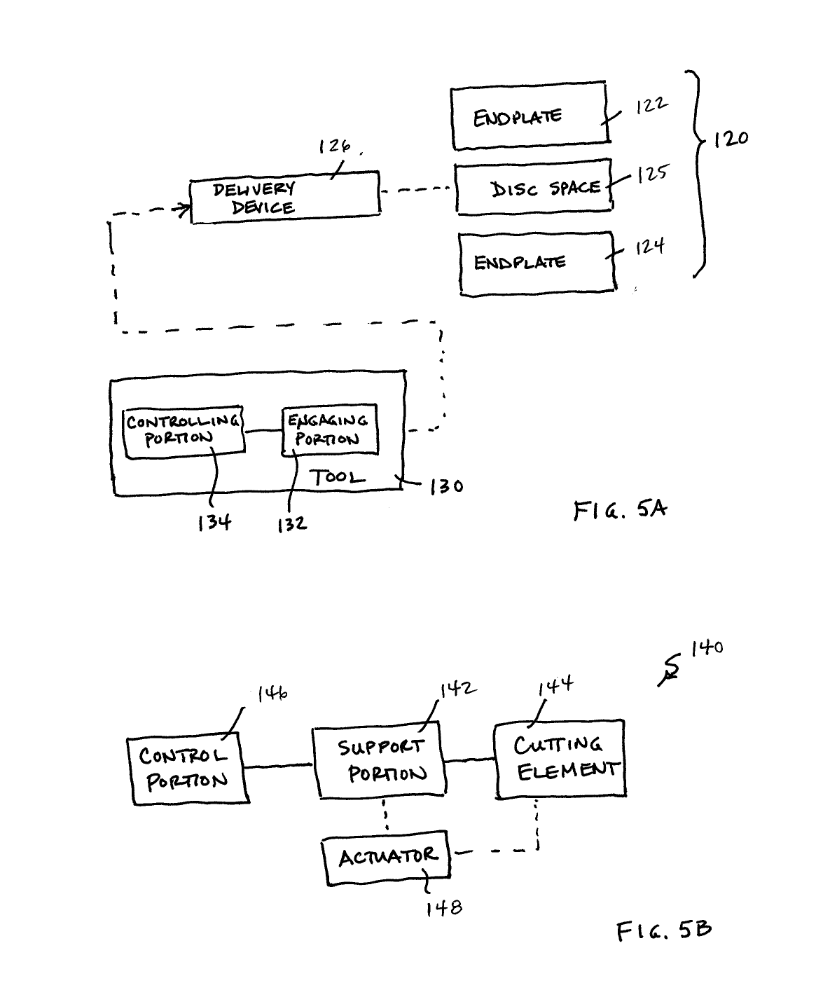

The engaging device 104 is inserted through the delivery device 102 and can be moved inwardly until it engages a target area or region, which can be one of the endplates. The engaging device 104 can be moved by the physician repeatedly along the directions of arrows “A” and “B” to engage the target area, which in the example illustrated in FIG. 3 is endplate 106. The engaging device 104 may include a sharp edge or point, or alternatively, may include a cutting element configured to cut or scrape the target area. In the embodiment shown in FIGS. 3 and 4, the engaging device 104 is illustrated as being a simple shaft 104 that can be moved to engage a target area. Various embodiments of tools, engaging devices, and cutting elements are illustrated in FIGS. 7-77 and described below.

The engaging device 104 is repeatedly moved until the physician believes that enough damage has been done to induce the flow of blood into the disc space 110. As illustrated in FIG. 4, the engaging device 104 has been used to scrap or break the endplate 106 in area 112 and cause the flow of blood 116 into the disc space 110. The device 104 can be used to penetrate the end plate 106 as well. To induce the flow of blood, while it is not required that the endplate 106 be broken through to the cancellous portion 114, that would be the easiest manner in which to achieve blood flow. In one exemplary method, the physician can withdraw the engaging device 104 through the delivery device 102 and inspect the engaging device 104 for the presence of blood. If not blood is present on the engaging device 104, the physician can re-insert the engaging device 104 and repeat the cutting or scraping process. When the process is complete, the engaging device 104 is withdrawn along the direction of arrow “C” and the delivery device 102 and engaging device 104 are removed from the patient.

The terms “cutting” and “scraping” are used interchangeably herein to mean the relative movement of one item against another to cause some level of damage to the item being engaged. The level of damage desired can vary depending on the goal of the physician. In the context of this invention, the cutting and scraping involves engaging part of a tool against an internal body component proximate to a disc space. Some other alternative terms that can be used in lieu of “cutting” or “scraping” can include “abrading,” “eroding,” and “traumatizing.” These terms may also be used interchangeably herein.

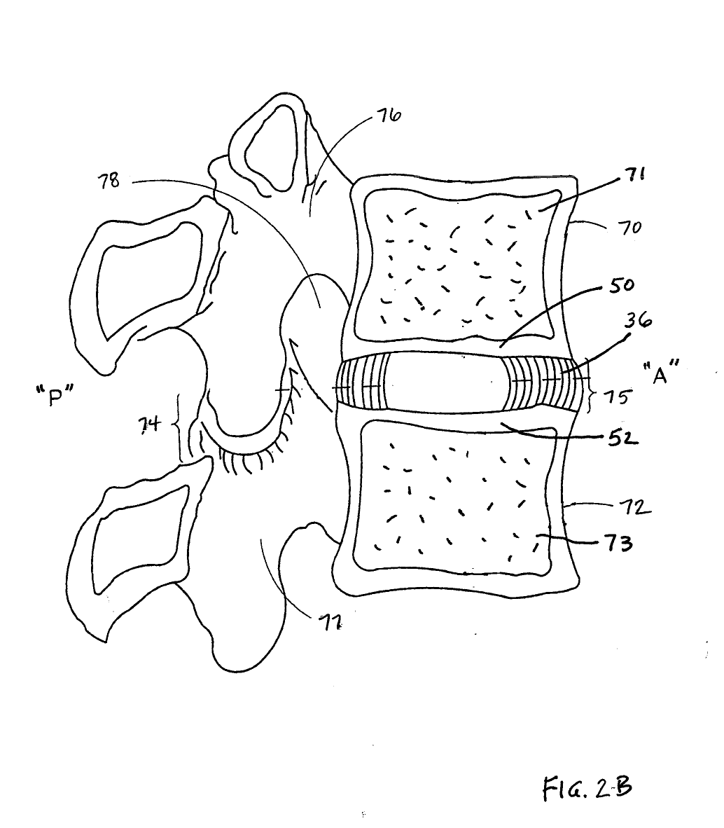

Some exemplary block diagrams of different embodiments of tools that can be used according to the invention are illustrated in FIGS. 5A and 5B. These embodiments are intended to be exemplary only and to illustrate some of the features that a tool according to the invention may include. As will be seen in the description of the various embodiments of tools illustrated in FIGS. 7-77, the components of the tools can vary. However, the basic aspects of a tool according to the invention are that the tool includes a surface or cutting element that is either part of or mounted to a support that can be manipulated by a physician either manually or using a mechanism.

Referring to FIG. 5A, an exemplary target area 120 comprising endplates 122 and 124 and a disc space 125 is illustrated. A delivery device 126 can be inserted into the patient's body and moved so that it extends into the disc space 125. Based on the minimal size of the delivery device 126, no incision or at most, a negligible skin incision, needs to be made to the patient's body to insert the delivery device 126. Further, no portion of the spinal column of the patient needs to be cut or removed to enable the delivery device 126 to access the disc space 125. The delivery device 126 may include a handle at its proximal end that a physician may use to insert and move the delivery device 126.

Also illustrated in FIG. 5A is a tool 130 that can be through the delivery device 126 and into the disc space 125. The tool 120 includes an engaging portion 132 and a controlling portion 134. The engaging portion 132 is the part of the tool that does the work and the controlling portion 134 is the part of the tool that enables a physician to move the engaging portion 132 in a desired manner.

A block diagram of an alternative embodiment of a tool according to the invention is illustrated in FIG. 5B. Tool 140 includes a support portion 142 and a cutting element 144 coupled to the support portion 142. In different embodiments, the cutting element 144 can be fixedly coupled to the support portion 142 or movably coupled to the support portion 142.

In one embodiment, the cutting element 144 can be integrally formed with the support portion 142. In that implementation, the cutting element 144 can be a point, a tip, or an edge that is formed on the support portion 142. In other embodiments, the cutting element 144 can be formed separately from the support portion 142 and coupled thereto.

In a different embodiment of tool 140, there may be more than one cutting element 144 coupled to the support portion 142. The amount of cutting or scraping that occurs with each movement of the tool 140 is determined by the amount of cutting or scraping area of the cutting element or elements and the quantity of the elements.

As illustrated in FIG. 5B, the tool 140 includes a control portion 146 that is coupled to the support portion 142. The control portion 146 may be manipulated by the physician manually or using a mechanism. Such manipulation allows the physician to control the movement of the cutting element 144 in the disc space. The control portion 146 can have any shape or configuration provided that the physician can grasp and manipulate the control portion 146 as desired. In some embodiments, the control portion 146 can include a handle.

In some embodiments of a tool according to the invention, such as tool 140 in FIG. 5B, the tool 140 may include an actuator 148 that is coupled to the cutting element 144 or elements. The actuator 148 is movable relative to the support portion 142 which allows it to adjust or move a cutting element from one configuration to another configuration relative to the support portion 142. Similar to the control portion 146, the actuator 148 can have any shape or configuration provided that it can be manipulated by a physician. In one implementation, the actuator 148 may have a handle that can be grasped and used by a physician.

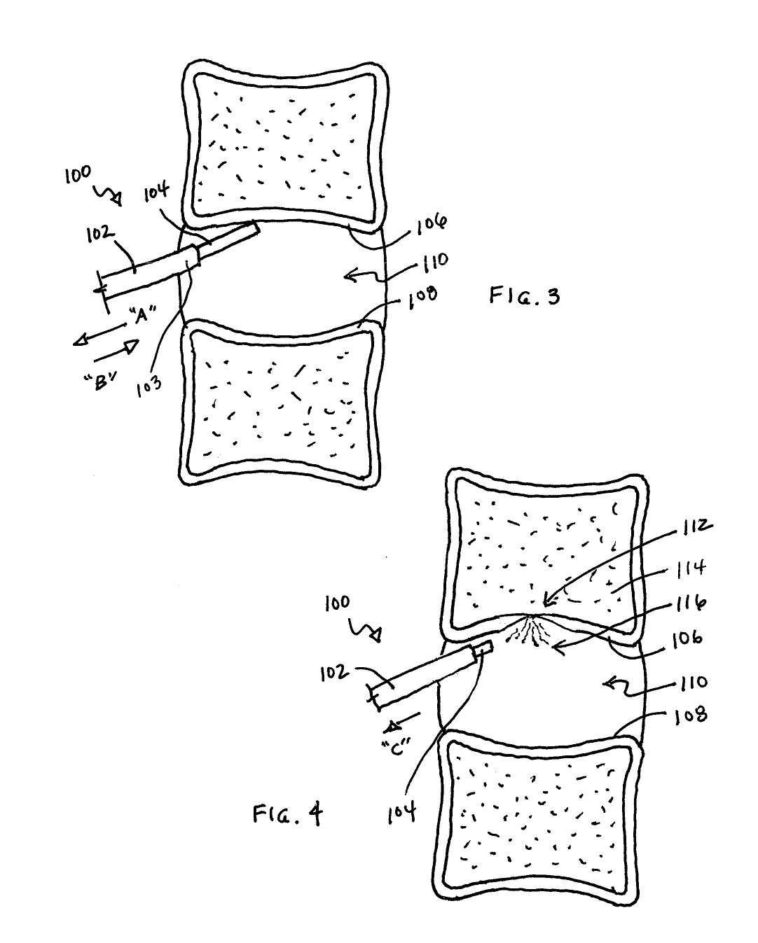

Some additional embodiments of tools are illustrated in FIGS. 6A-6C. Referring to FIG. 6A, the tool 160 includes a delivery device 162 through which a preparation or engaging device 163 can be inserted. The engaging device 163 includes a support portion or shaft 164 with a proximal end 166 and a distal end 168. A control portion 170 is coupled to the proximal end 166 of the shaft 164 and is configured to be grasped by a physician. In one embodiment, the control portion 170 is a bar that forms a T-shaped handle with the shaft 164. In another embodiment, the control portion 170 can have a disc-shaped configuration. The distal end 168 can be formed to include a cutting point or tip or alternatively, it can have one or more cutting elements (not shown) coupled thereto. The engaging device 163 can move within the delivery device 162 along the directions of arrows “A” and “B” in FIG. 6A.

Referring to FIG. 6B, tool 180 includes a support portion or shaft 182 with a control portion 186 coupled to one end and an engaging portion 184 formed at its opposite end. The control portion 186 is a loop-shaped handle that has a central opening 188 that facilitates the grasping of the control portion 186. Tool 180 may include a stop 189 to check or limit the depth to which the tool is inserted into the patient. The stop 189 may be a structural limitation formed on the support. Alternatively, the stop may be a ring or clip 189 that can be added to the shaft of the tool when the physician determines that the end of the disc space on the anterior side has been reached by the tool. The clip or ring 189 provides a visual indicator of the limitation of the depth that the tool should be inserted. The stop can be a snap-on or clip-on structure that can be removed from the shaft after a process. The stop can be used with any of the tools described herein and can have different shapes, configurations, and colors in different embodiments.

Referring to FIG. 6C, tool 190 includes a support portion or shaft 192 that has an engaging portion 194 formed at one end and a longitudinal axis 198. In this embodiment, a drive mechanism 196, such as a motor, can be coupled to one end of the shaft 192 to rotate the shaft 192 about its longitudinal axis 198 along the direction of arrow “C1” in FIG. 6C. Alternatively, the drive mechanism 196 can be coupled to one end of the shaft 192 to impart reciprocating, linear movement of the shaft 192 along the directions of arrows “A1” and “B1.” The drive mechanism 196 can have any shape or configuration and can be coupled to the shaft of a tool using any conventional drive components, such as gears, drive wheels, pulleys or the like, provided that movement can be imparted to the shaft by the drive mechanism. The drive mechanism 196 can be powered by an internal or an external power supply and can be controlled directly or indirectly by a physician or other individual.

Now, numerous alternative embodiments of tools that can used in the processes and methods disclosed herein will be described. It is to be understood that features of different embodiments of tools may be combined together and used in other tool embodiments, which are encompassed as part of the tools of the invention.

An embodiment of a tool according to the invention is illustrated in FIGS. 7-8. In this embodiment, tool 200 includes several cutting elements. The cutting elements are configured to be inserted into a disc space and subsequently moved to engage a targeted treatment area, such as an end plate.

Referring to FIG. 7, the tool 200 includes a shaft 202 that has a proximal end 204, a distal end 206, and a longitudinal axis 205. The proximal end 204 is the end of the shaft 202 proximate to the user of the tool 200. A controlling portion, such as a handle, can be coupled to the proximal end 204 so that a user can easily manipulate and use the tool 200. Examples of controlling portions are described in detail below.

The shaft 202 can be made of a flexible material, such as stainless steel, nickel-titanium alloys (NITINOL material), and other metal alloys. In this embodiment, the shaft 202 has a substantially cylindrical configuration. However, in alternative embodiments, the shaft can have different shaped configurations.

In this embodiment, the shaft 202 has two portions. The portion of the shaft 202 without the cutting elements can be referred to as a support portion 207 and the portion with the cutting elements can be referred to as a cutting or engaging portion 209. The engaging portion 209 is located proximate to the distal end 206 of the shaft 202. As illustrated in FIG. 7, the support portion 207 and the engaging portion 209 of the shaft 202 can be integrally formed as a single piece. In alternative embodiments, separate support and engaging portions can be formed and subsequently coupled together to form the shaft.

The shaft 202 includes several bundles of cutting elements. The bundles 208A, 208B, 208C, and 208D are bundles of cutting elements 210, such as filaments or bristles, that are coupled to the shaft 202 at spaced apart locations. In this embodiment, four bundles are coupled to the shaft 202. In alternative embodiments, the tool may include any number of bundles coupled to the shaft.

Each bristle 210 extends substantially radially from the shaft 202 from an end 212. Referring to FIG. 7, each bristle 210 is illustrated in a first configuration or position 214, which can be referred to as a deployed position. The bristles 210 can be made from a resilient stainless steel, an injection molded inert plastic, or a shape memory material, like NITINOL. The cross-sectional configuration of the bristles 210 can be round, rectilinear, or any other configuration.

As illustrated in FIG. 8, the tool 200 is introduced into the targeted region through a delivery device 220 along the direction of arrow “D.” The delivery device 220 can be a needle or cannula. When the tool 200 is in the delivery device 220, the resilient bristles 210 are compressed rearwardly by the inner surface of the delivery device 220. This second configuration or position 216, which is a delivery configuration, facilitates the passage of the tool 200 through the delivery device 220. As shown, when a bundle of bristles exits the distal end 222 of the delivery device 220, the resilient nature of the bristles 210 causes them to spring outwardly and return to their deployed positions 214. Bundle 208D is illustrated as being in its deployed configuration or position 214 and ready for use.

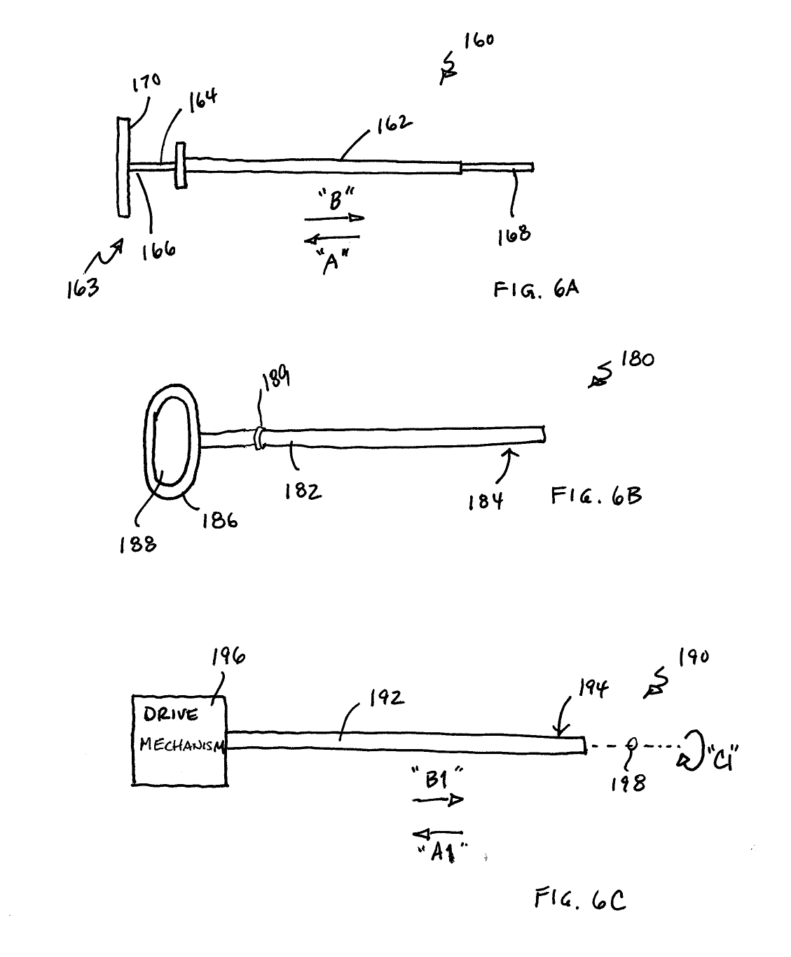

An alternative embodiment of a tool is illustrated in FIG. 9. The tool 230 includes a shaft 232 and several bundles of cutting elements. The cutting elements are similar to the cutting elements 210 described above for tool 200. In this embodiment, the tool 230 includes bundles 240A, 240B, 240C, 240D, and 240E. The shaft 232 has a support portion 234 and an engaging portion 236, which in this implementation, are integrally formed.

In this embodiment, tool 230 is flexible and has a shape-changing behavior. As illustrated in FIG. 9, the tool 230 has a first, deployed configuration 242. In this configuration 242, the engaging portion 236 extends or projects away from the longitudinal axis 261 of the shaft 232. This configuration 242 represents an initial or undeformed state of the shaft 232.

The tool 230 is configured to pass telescopically through the interior of a delivery device, such as delivery device 220 described above. As the tool 230 is inserted into the delivery device, the engaging portion 236 experiences elastic deformation, such as being spring loaded, and assumes a second, delivery or deformed configuration 244 in which the engaging portion 236 is substantially linear with the support portion 234 and co-linear with the longitudinal axis 261.

As the engaging portion 236 extends beyond the end of the delivery device, the spring bias arising from elastic deformation tends to move the engaging portion 236 of the shaft 232 from configuration 244 toward configuration 242 along the direction of arrow “E.” The engaging portion 236 seeks to return to configuration 242 because it is a spring unloaded configuration. By reversing the insertion process, the tool 230 can be removed through the delivery device.

The shaft 232 of tool 230 can be constructed from a variety of appropriate stainless steels capable of elastic behavior. Consistent with spring mechanics, the shape change of the engaging portion 236 of the shaft 232 should be within the elastic range of the material. Another suitable material is the metal alloy NITINOL, a biomaterial capable of superelastic mechanical behavior, meaning that the material can recover from significantly greater deformation as compared to most other metal alloys. The NITINOL metal alloy contains almost equal parts of titanium and nickel. Alternatively, the shaft 232 can be constructed from a polymer, such as nylon or ultra high molecular weight polyethylene.

A thermal shape-memory alloy can also be used for biasing a portion of the shaft to move from a first configuration to a second configuration. The most commonly used biomaterial with thermal shape-memory properties is the NITINOL metal alloy. A flexible cutting element that is constructed from NITINOL can be deformed below a transformation temperature to a shape suitable for percutaneous placement into tissue. The reversal of deformation of the element is achieved when the element is heated through the transformation temperature. The applied heat can be from the surrounding tissue, or associated with frictional heat generated during operation. NITINOL is capable of a wide range of shape-memory transformation temperatures appropriate for the clinical setting. In an alternative embodiment, heat may be applied by passing an electrical current through the material to cause resistive heating.

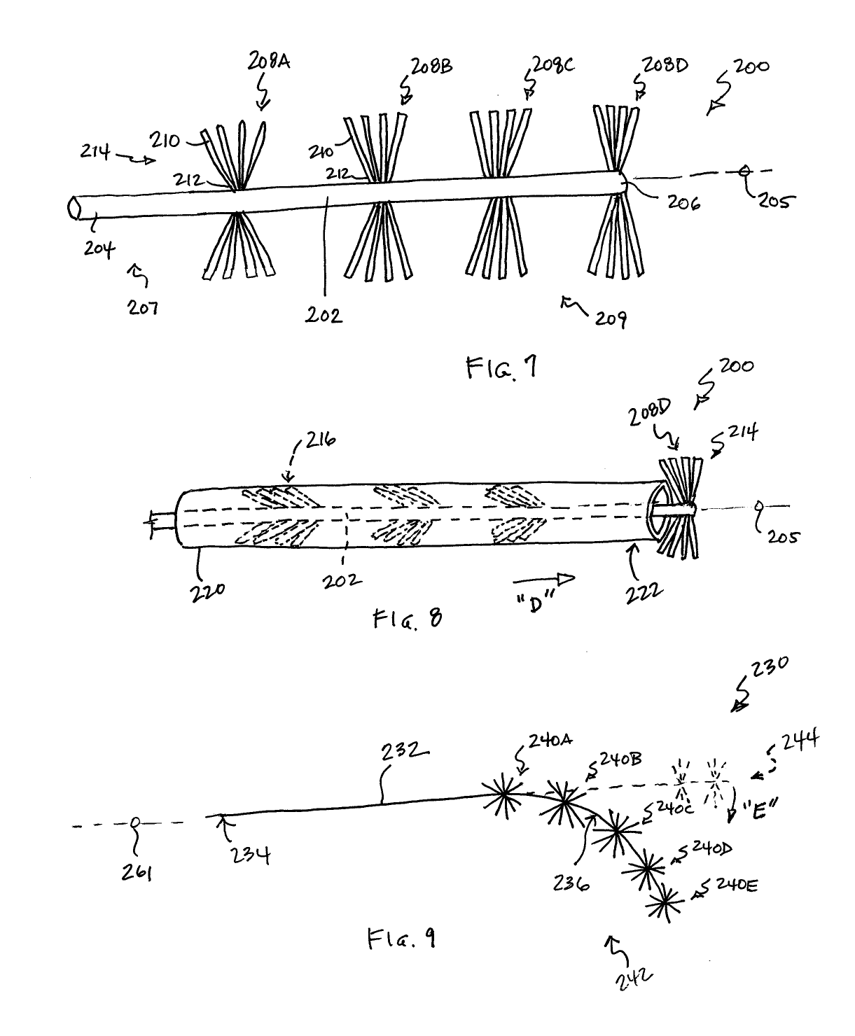

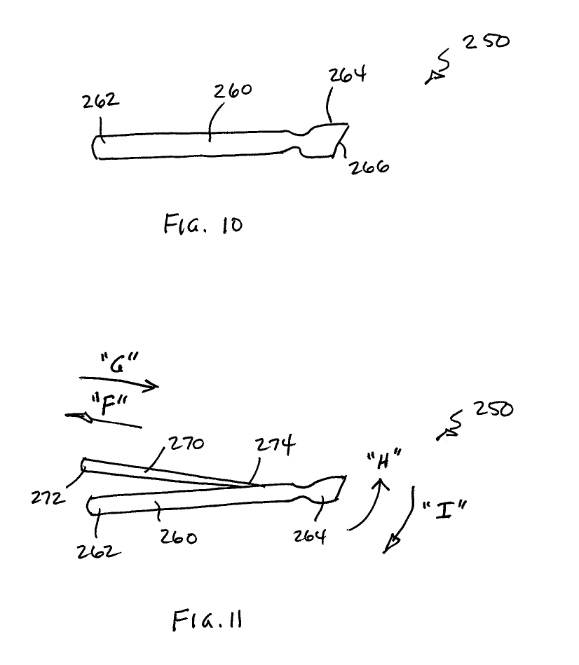

An alternative embodiment of a tool is illustrated in FIGS. 10-11. In this embodiment, the tool 250 includes a shaft 260 and a control element 270 that is coupled to the shaft 260. The shaft 260 has a proximal end 262 and a distal end 264 and is formed of a flexible material. Adjacent the distal end 264 is a cutting edge or tip 266. The cutting edge 266 is sufficiently sharp or abrasive to scrape or cut an endplate.

A control element or actuator 270 is coupled to the shaft 260 and can be manipulated by a user. The control element 270 includes a proximal end 272 and a distal end 274. The distal end 274 of the control element 270 is coupled to the shaft 260 proximate to the distal end 264 of the shaft 260. The coupling can be achieved by fusing the end of the control element 270 to the shaft 260. Alternatively, any conventional type of connector or adhesive can be used.

As a user moves the control element 270 along the direction of arrow “F,” the distal end 264 of the shaft 260 bends and moves along the direction of arrow “H.” When the force applied to the control element 270 is released, the biasing force of the shaft 260 causes the distal end 264 to return to its initial position and move along the direction of arrow “I.” As a result, the control element 270 is moved along the direction of arrow “G.” The control element 270 can be moved back and forth and thereby cause the cutting edge 266 to repeatedly scrape or cut a particular surface.

In one embodiment, the movement of the control element 270 can be performed manually by the operator of the tool 250. In alternative embodiments, the control element 270 can be manipulated by mechanical means.

An alternative embodiment of a tool according to the invention is illustrated in FIGS. 12-16. Tool 300 is exemplary of a tool that can be inserted through a delivery device, such as a needle or cannula, to be deployed in a disc space. Tool 300 can manipulated by a user to engage a superior endplate and/or inferior endplate in a disc space.

Tool 300 includes a shaft 310 with a proximal end 302 and an opposite, distal end 304. In this embodiment, the shaft 310 is a tube with an outer surface 312 and an inner surface 314 that defines a channel 316 extending therethrough. The shaft 310 is substantially cylindrical and can be passed through a delivery device.

As illustrated in FIG. 13, the shaft 310 includes a cutting region or portion 330. As will be described in detail below, the cutting region 330 is adjustable and can be manipulated to engage a target region in the disc space. Several slits 332 are formed in the shaft 310 around the perimeter. The slits 332 can be formed using a material cutting process, such as Electric Discharge Machining (“EDM”). The slits 332 extend from the outer surface 312 through to the inner surface 314 and extend between ends 334 and 336.

In the cutting region 330, a cutting element or member 340 is formed between each pair of slits 332. The width of the cutting members 340 are determined by the spacing of the slits 332 around the perimeter of the shaft 310.

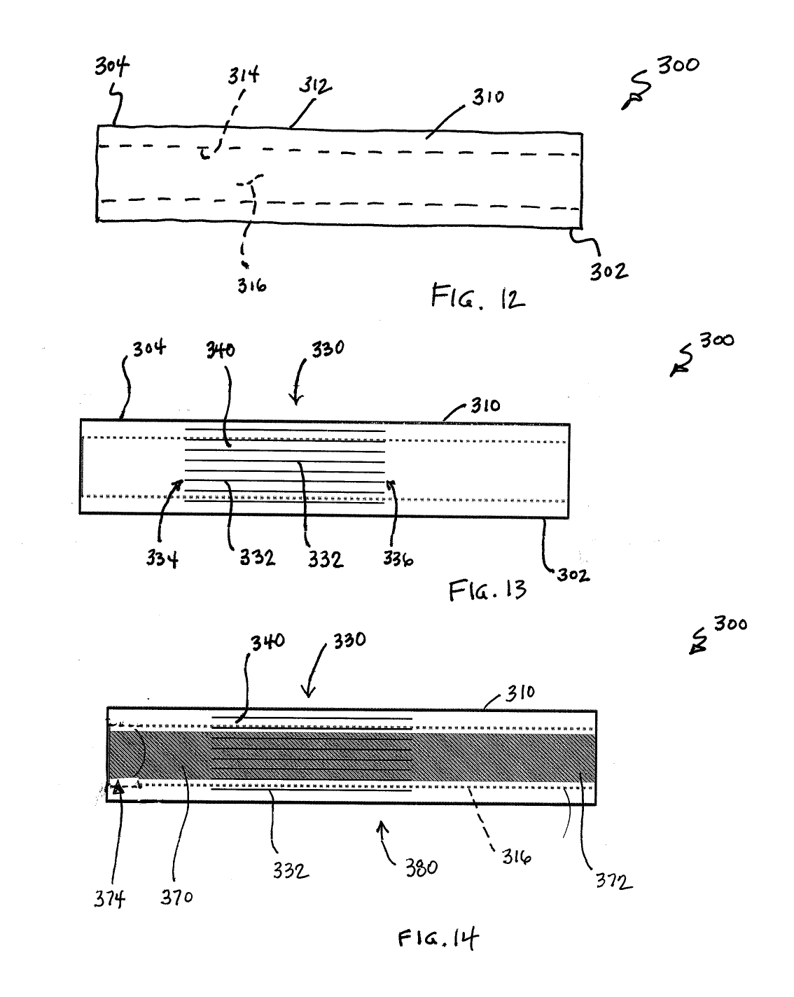

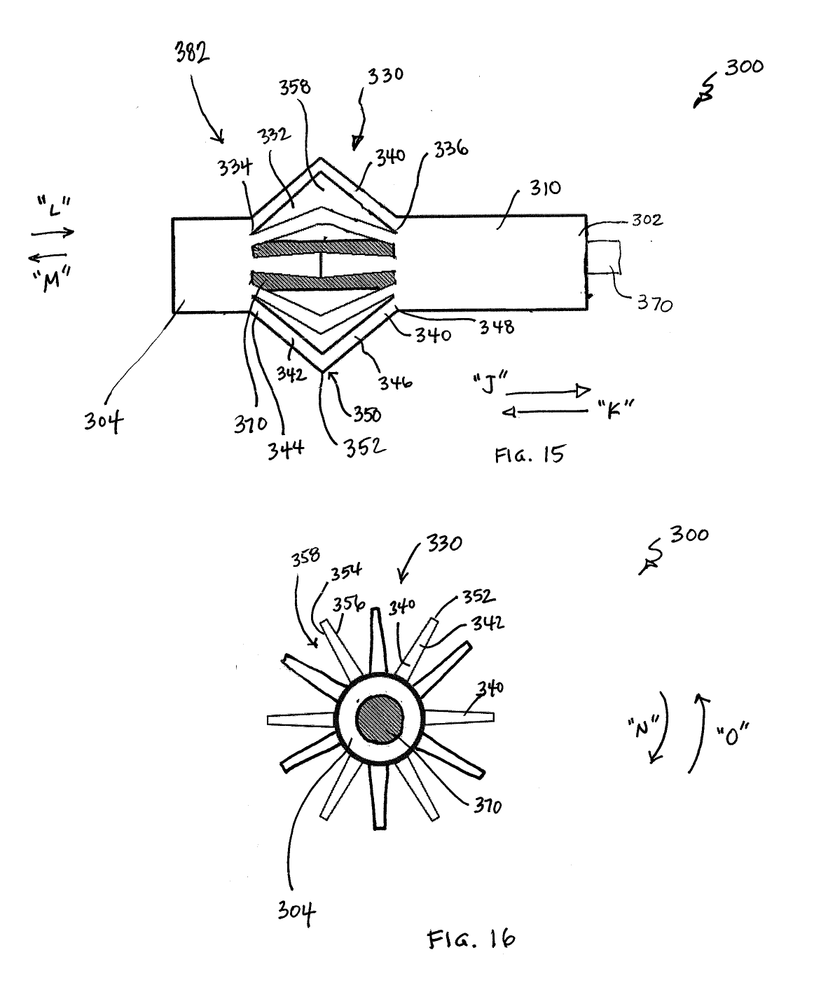

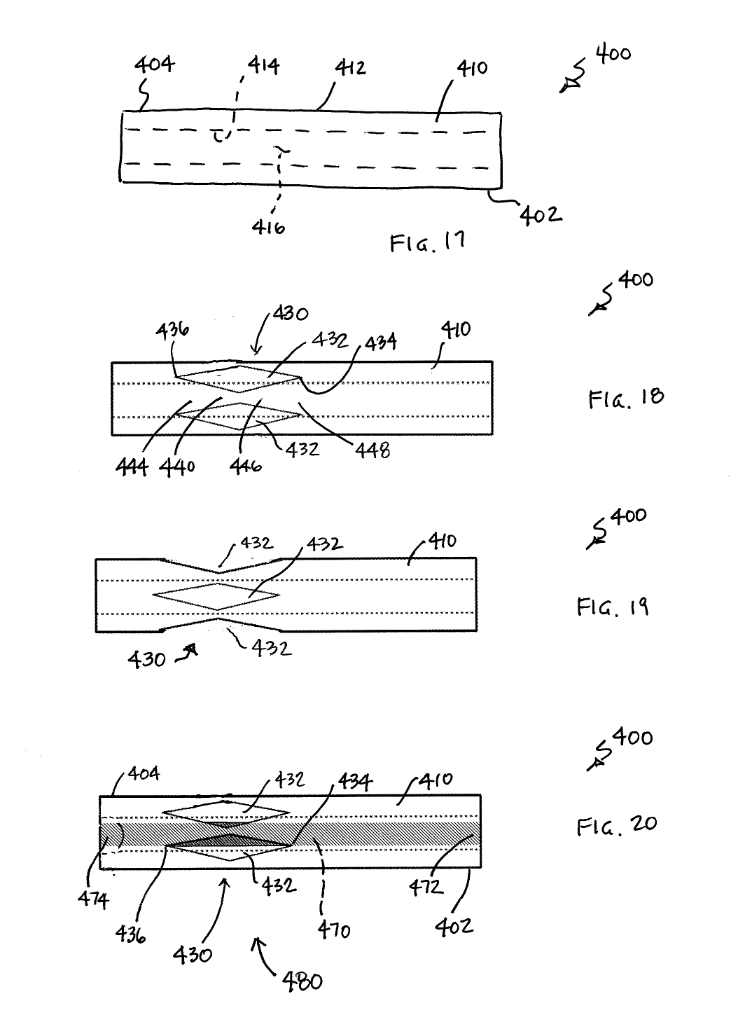

Referring to FIG. 14, after the slits 332 have been made in the shaft 310, an actuator 370 is inserted into the channel 316 of the shaft 310. The actuator 370 has a proximal end 372 and a distal end 374. In this embodiment, the distal end 374 of the actuator 370 is coupled to the shaft 310 proximate to end 304. The proximal end 372 of the actuator 370 is not coupled to the shaft 310 and can be manipulated by a user. The actuator 370 is dimensioned so that the actuator can slide within the channel 316. In this embodiment, the actuator 370 is a substantially cylindrical rod. However, in alternative embodiments, the actuator may have different cross-sectional configurations.

The cutting region 330 of the shaft 310 is illustrated in a delivery or unbiased configuration 380 in FIG. 14. In this configuration 380, the cutting elements 340 are stretched out and are disposed within the substantially cylindrical profile of the shaft 310. In other words, the cutting elements 340 do not extend outwardly beyond the original cylindrical shape of the shaft 310.

Referring to FIG. 15, an exemplary method of adjusting the tool 300 is illustrated. Adjustment of the tool 300 occurs after the tool 300 has been deployed through a delivery device and the cutting region 330 of the tool 300 is located proximate to the target area in a disc space. The tool 300 can be adjusted so that the cutting region 330 is in an expanded or deployed configuration 382 as illustrated in FIG. 15.

As previously mentioned, the proximal end 372 of the actuator 370 can be manipulated or moved relative to the shaft 310. The movement of the actuator 370 relative to the shaft 310 causes the distal end 304 of the shaft 310 to move relative to the proximal end 302 of the shaft 310, thereby causing the shape or configuration of the cutting region 330 to change.

For example, the actuator 370 can be moved along the direction of arrow “J.” Movement along that direction causes the distal end 304 of the shaft 310 to move in the same direction. As the distal end 304 moves, the cutting elements 340 spread apart as illustrated in FIG. 15 because the slits 332 were formed in the cutting region 330. Each cutting element 340 includes a first portion 342 with an end 344 and a second portion 346 with an end 348. When the cutting elements 340 are spread apart, each of the first portion 342 and the second portion 344 can have a curved configuration or as shown in this embodiment, can be substantially linear. Between adjacent cutting elements 340 a space 358 is formed and defined by sides 354 and 356 of the cutting elements 340.

When the cutting region 330 is expanded, an engaging area 350 is formed between the first portion 342 and the second portion 346. In this embodiment, the engaging area 350 forms a point or a tip 352 which can be used to cut or scrape a target area. The distance that the cutting elements 340 extend outwardly from the shaft 310 is determined by the distance that the actuator 370 is moved along the direction of arrow “J.” An end view of the tool 300 with the cutting elements 340 extending outwardly is illustrated in FIG. 16.

When the cutting region 330 is disposed in its expanded or deployed configuration 382, the tool 300 can be manipulated so that the cutting region 330 engages the target area, such as a superior endplate or an inferior endplate. For example, the shaft 310 and the actuator 370 together can be moved back and forth along the directions of arrows “L” and “M” as shown in FIG. 15. This movement can allow the cutting region 330 to scrape or cut the endplate. In addition, the shaft 310 and the actuator 370 can be rotated along the longitudinal axis of the shaft 310 along the directions of arrows “N” and “O” as shown in FIG. 16.

When the process of cutting or scraping the endplates or facet joint articulating surfaces has been completed, the tool 300 can be manipulated to return to its collapsed or delivery configuration. To collapse the cutting region 330, the actuator 370 is moved relative to the shaft 310 along the direction of arrow “K” in FIG. 15. When the actuator 370 moves the distal end 304 of the shaft 310 to its farthest position, the cutting elements 340 will be linear and disposed within the cylindrical configuration or profile of the shaft 310.

An alternative embodiment of a tool according to the invention is illustrated in FIGS. 17-23. In this embodiment, tool 400 can be inserted through a delivery device and deployed in a disc space to induce the flow of blood into the disc space. The tool 400 can be manipulated by a user to engage a superior endplate and/or an inferior endplate in a disc space or articulating surface in a facet joint.

In this embodiment, tool 400 includes a shaft 410 with a proximal end 402 and an opposite, distal end 404. Similar to shaft 310, shaft 410 is a tube with an outer surface 412 and an inner surface 414 that defines a channel 416 extending through the shaft 410. The shaft 410 has a substantially cylindrical cross-sectional configuration.

As illustrated in FIG. 18, the shaft 410 includes a cutting region or portion 430. The cutting region 430 is adjustable and the tool 400 can be manipulated so that the cutting region 430 engages a target region in the disc space.

As illustrated in FIGS. 18 and 19, several openings 432 are formed in the shaft 410 around its perimeter in the cutting region 430. FIG. 18 illustrates a top view of the shaft 410 and FIG. 19 illustrates a side view of the shaft 410. The openings 432 can be formed using a material cutting process, such as EDM. The openings 432 extend from the outer surface 412 through to the inner surface 414 and extend between ends 434 and 436. In this embodiment, the openings 432 have a diamond shapes and can be referred to as notches.

In the cutting region 430, a cutting element or member 440 is formed between adjacent pairs of openings 432. The width of the cutting members 440 are determined by the spacing of the openings 432 around the perimeter of the shaft 410.

Referring to FIG. 20, after the openings 432 have been made in the shaft 410, an actuator 470 is inserted into the channel 416 of the shaft 410. The actuator 470 has a proximal end 472 and a distal end 474 which is coupled to the shaft 410 proximate to shaft end 404. The proximal end 472 of the actuator 470 is not coupled to the shaft 410 and can be manipulated by a user. Actuator 470 is a substantially cylindrical rod, but in other embodiments, it may have different cross-sectional configurations.

The cutting region 430 of the shaft 410 is illustrated in a delivery or unbiased configuration 480 in FIGS. 20 and 21. In this configuration 480, the cutting elements 440 are stretched out and are disposed within the substantially cylindrical profile of the shaft 410. In other words, the cutting elements 440 do not extend outwardly beyond the original cylindrical shape of the shaft 410.

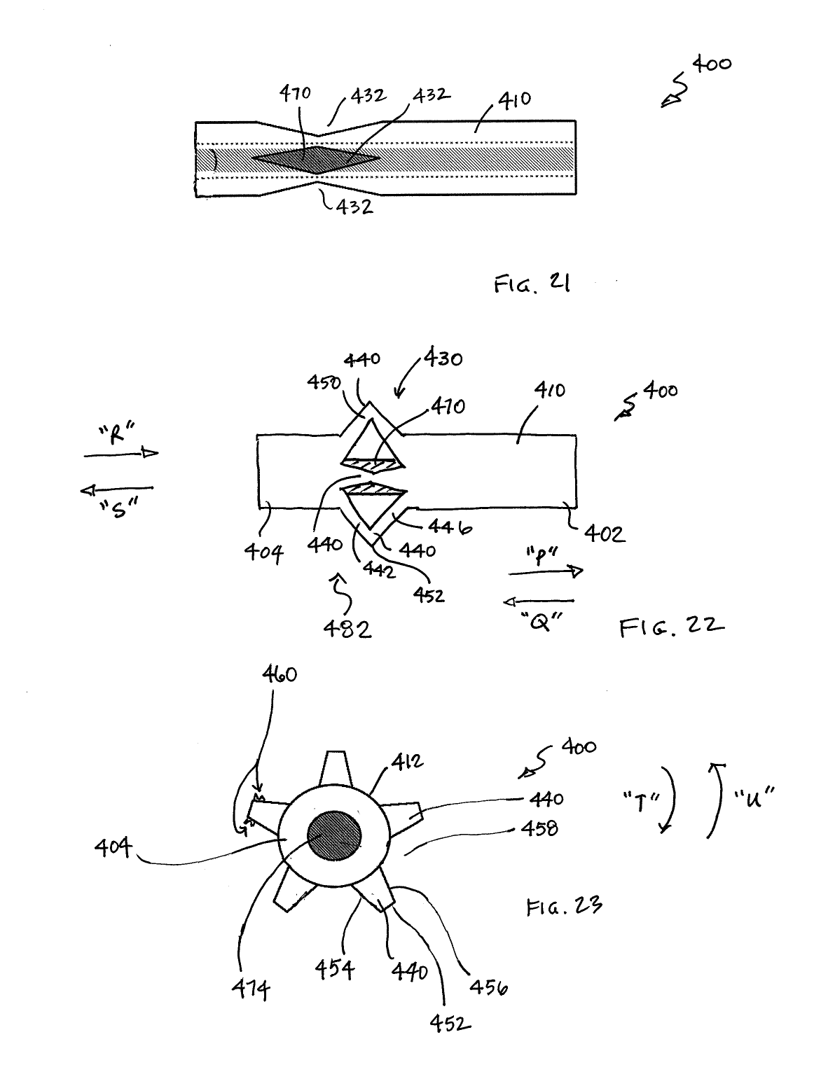

Referring to FIG. 22, an exemplary method of adjusting the tool 400 is illustrated. Adjustment of the tool 400 occurs after the tool 400 has been deployed through a delivery device and the cutting region 430 of the tool 400 is located proximate to the target area in a disc space. The tool 400 can be adjusted so that the cutting region 430 is in an expanded or deployed configuration 482 as illustrated in FIGS. 22 and 23.

The proximal end 472 of the actuator 470 can be manipulated or moved relative to the shaft 410. The movement of the actuator 470 relative to the shaft 410 causes the distal end 404 of the shaft 410 to move relative to the proximal end 402 of the shaft 410, thereby causing the shape or configuration of the cutting region 430 to change.

The actuator 470 can be moved along the direction of arrow “P” in FIG. 22 and such movement causes the distal end 404 of the shaft 410 to move in the same direction. As the distal end 404 moves toward the proximal end 402, the cutting elements 440 spread apart as illustrated in FIG. 22 because the slits 432 were formed in the cutting region 430. Each cutting element 440 includes a first portion 442 with an end 444, a second portion 446 with an end 448, and sides 454 and 456. Adjacent cutting elements 440 have a space 458 between them. When the cutting elements 440 are spread apart, each of the first portion 442 and the second portion 446 can have a curved configuration or as shown in this embodiment, can be substantially linear.

When the cutting region 430 is expanded, an engaging area 450 is formed between the first portion 442 and the second portion 446. In this embodiment, the engaging area 450 forms a point or a tip 452 which can be used to cut or scrape a target area. The distance that the cutting elements 440 extend outwardly from the shaft 410 is determined by the distance that the actuator 470 is moved along the direction of arrow “P.” An end view of the tool 400 with the cutting elements 440 extending outwardly is illustrated in FIG. 23.

When the cutting region 430 is disposed in its expanded or deployed configuration 482, the tool 400 can be manipulated so that the cutting region 430 engages the target area, such as a superior endplate or an inferior endplate. For example, the shaft 410 and the actuator 470 together can be moved back and forth along the longitudinal axis of the shaft 410 along the directions of arrows “R” and “S” as shown in FIG. 22. This movement allows the cutting region 430 to engage and scrape or cut an endplate. In addition, the shaft 410 and the actuator 470 can be rotated along the longitudinal axis of the shaft 410 along the directions of arrows “T” and “U” as shown in FIG. 23.

When the process of cutting or scraping the endplates or facet joint surfaces has been completed, the tool 400 can be manipulated to return to its collapsed or delivery configuration. To collapse the cutting region 430, the actuator 470 is moved relative to the shaft 410 along the direction of arrow “Q” in FIG. 22. When the actuator 470 moves the distal end 404 of the shaft 410 to its farthest position, the cutting elements 440 will be linear and disposed within the cylindrical configuration or profile of the shaft 410.

In this embodiment, several abrasive pieces 460 are coupled to the sides 454 and 456 of the cutting elements 440. The abrasive pieces 460 can be adhered to the sides 454 and 456 using any conventional method or technique. The abrasive pieces 460 improve the cutting and scraping action of the cutting elements 440 during use. If the openings 432 are dimensioned sufficiently, the abrasive pieces 460 on adjacent cutting elements 440 will not contact each other when the cutting elements are in their collapsed configurations.

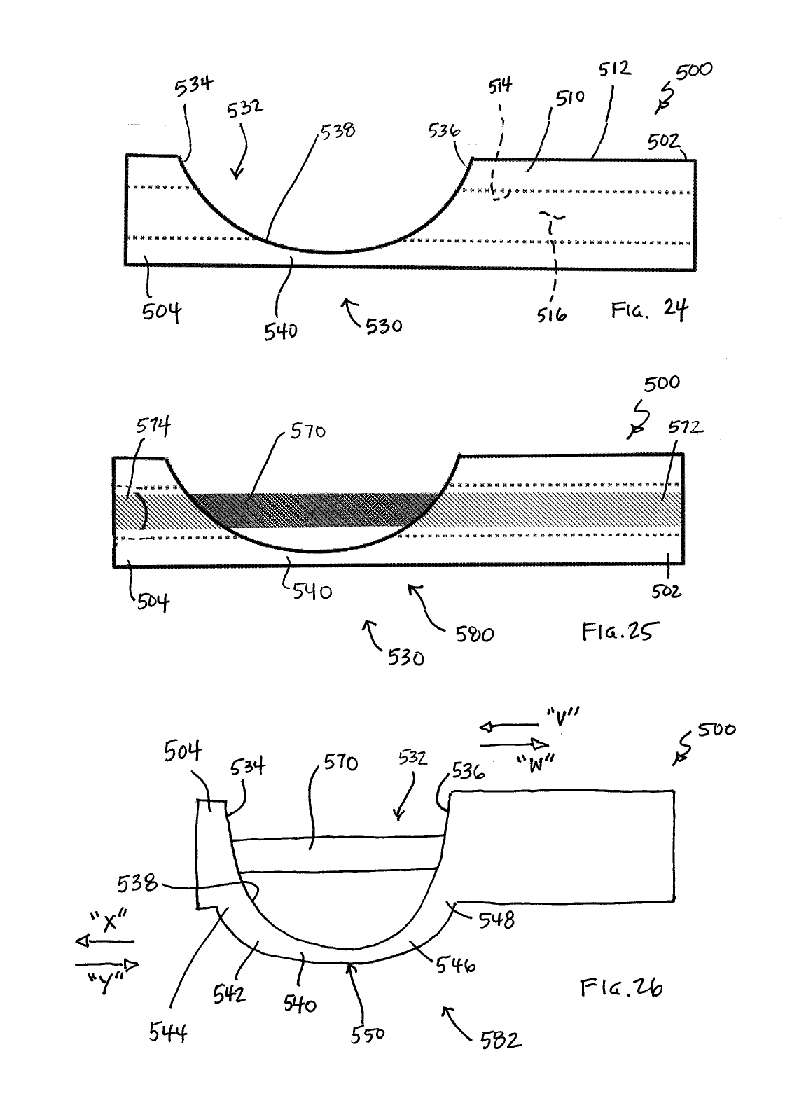

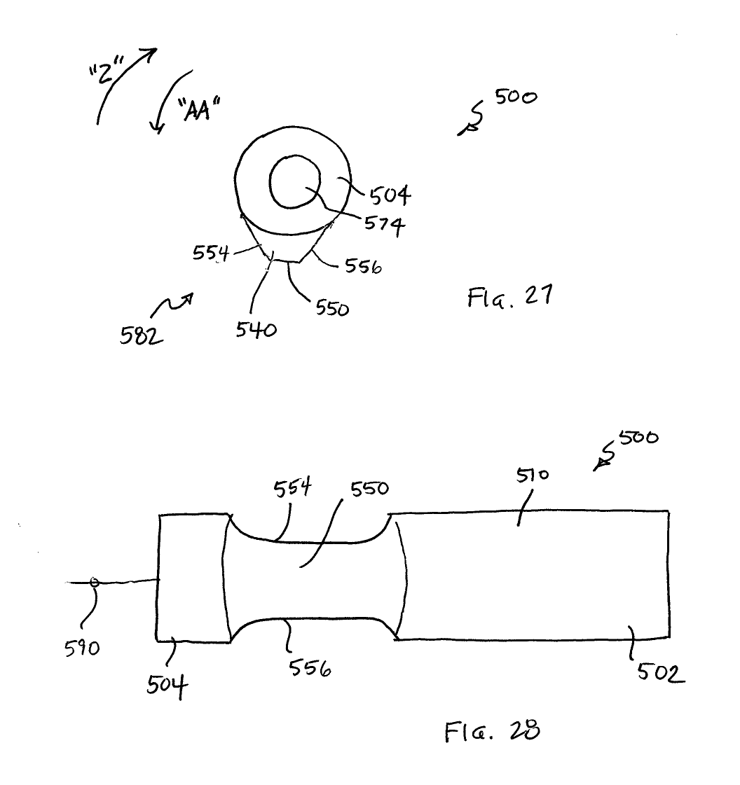

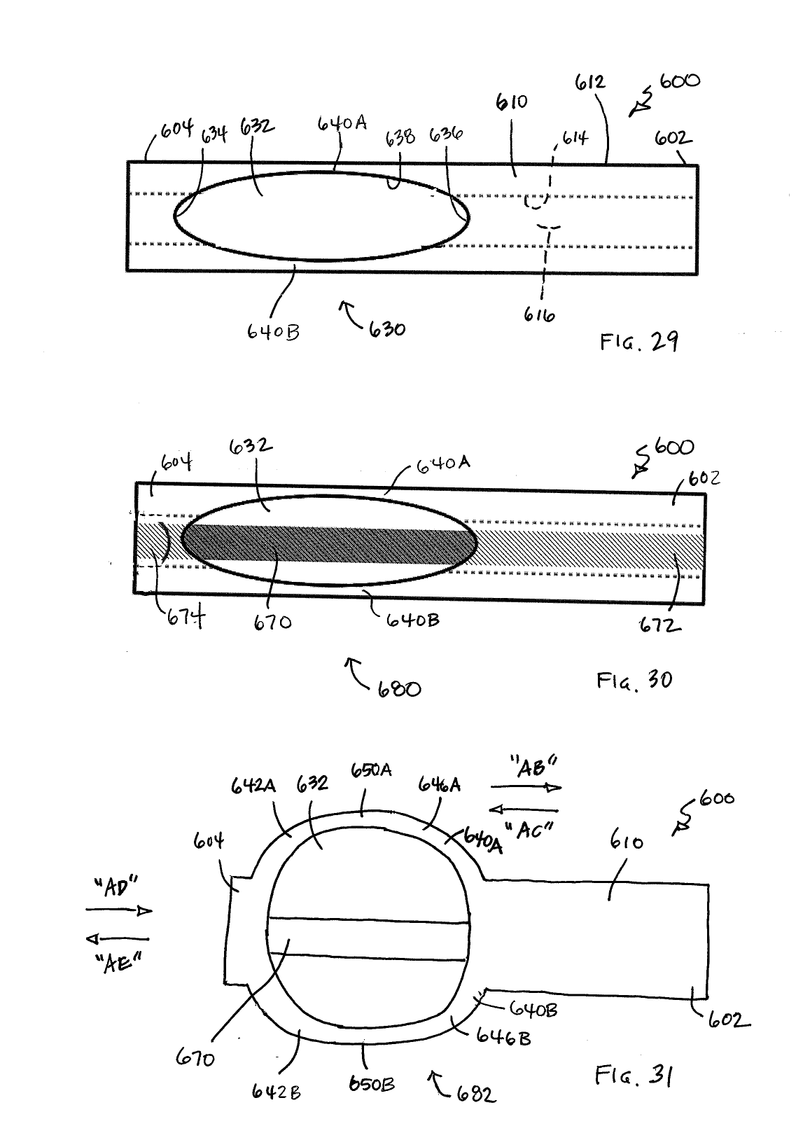

An alternative embodiment of a tool according to the invention is illustrated in FIGS. 24-28. In this embodiment, tool 500 can be inserted through a delivery device and deployed in a disc space or facet joint to induce the flow of blood into the disc space or facet joint. The tool 500 can be manipulated by a user to engage a superior endplate and/or an inferior endplate in a disc space.

In this embodiment, tool 500 includes a shaft 510 with a proximal end 502 and an opposite, distal end 504. Similar to shafts 310 and 410, shaft 510 is a tube with an outer surface 512 and an inner surface 514 that defines a channel 516 extending through the shaft 510. The shaft 510 has a substantially cylindrical cross-sectional configuration.

As illustrated in FIG. 24, the shaft 510 includes a cutting region or portion 530. The cutting region 530 is adjustable and the tool 500 can be manipulated so that the cutting region 530 engages a target region in the disc space.

As illustrated in FIG. 24, an opening or recess 532 is formed in the shaft 510 in the cutting region 530. The opening 532 can be formed using a material cutting process, such as EDM. The opening 532 extends substantially through the majority of the cutting region 530 and is defined by surface 538 that extends between ends 534 and 536. The opening 532 is in communication with the channel 516 of the shaft 510.

In the cutting region 530, a cutting element or member 540 is formed by the remaining material of the shaft 510 in the cutting region 530. The size of the cutting member 540 is determined by the dimension of the opening 532 formed in the cutting region 530.

Referring to FIG. 25, after the opening 532 has been made in the shaft 510, an actuator 570 is inserted into the channel 516 of the shaft 510. The actuator 570 has a proximal end 572 and a distal end 574 which is coupled to the shaft 510 proximate to shaft end 504. The proximal end 572 of the actuator 570 is not coupled to the shaft 510 and can be manipulated by a user. Actuator 570 is a substantially cylindrical rod, but in other embodiments, it may have different cross-sectional configurations.

The cutting region 530 of the shaft 510 is illustrated in a delivery or unbiased configuration 580 in FIGS. 24 and 25. In this configuration 580, the cutting element 540 is stretched out and is disposed within the substantially cylindrical profile of the shaft 510. Accordingly, the cutting element 540 does not extend outwardly beyond the original cylindrical shape of the shaft 510.

Referring to FIG. 26, an exemplary method of adjusting the tool 500 is illustrated. Adjustment of the tool 500 occurs after the tool 500 has been deployed through a delivery device and the cutting region 530 of the tool 500 is located proximate to the target area in a disc space. The tool 500 can be adjusted so that the cutting region 530 is in an expanded or deployed configuration 582 as illustrated in FIGS. 26-28. FIG. 26 illustrates a side view of the tool 500, FIG. 27 illustrates an end view of the tool 500, and FIG. 28 illustrates a bottom view of the tool 500.

The proximal end 572 of the actuator 570 can be manipulated or moved relative to the shaft 510. The movement of the actuator 570 relative to the shaft 510 causes the distal end 504 of the shaft 510 to move relative to the proximal end 502 of the shaft 510, thereby causing the shape or configuration of the cutting region 530 to change.

The actuator 570 can be moved along the direction of arrow “W” in FIG. 26 and such movement causes the distal end 504 of the shaft 510 to move in the same direction. As the distal end 504 moves toward the proximal end 502, the cutting element 540 bows or expands outwardly as illustrated in FIG. 26 because that part of the shaft 510 is the weakest portion. The cutting element 540 includes a first portion 542 with an end 544 and a second portion 546 with an end 548. When the cutting element 540 is expanded outwardly, its first portion 542 and its second portion 544 can have a curved configuration as shown in this embodiment, or alternatively, can be substantially linear.

When the cutting region 530 is expanded, an engaging area 550 is formed between the first portion 542 and the second portion 546. As illustrated in FIG. 27, the engaging area 550 includes sides 554 and 556 that have sharp edges that can be used to cut or scrap an endplate. The distance that the cutting element 540 extends outwardly from the shaft 510 is determined by the distance that the actuator 570 is moved along the direction of arrow “W” in FIG. 26. An end view of the tool 500 with the cutting element 540 extending outwardly is illustrated in FIG. 27.

When the cutting region 530 is disposed in its expanded or deployed configuration 582, the tool 500 can be manipulated so that the cutting region 530 engages the target area, such as a superior endplate or an inferior endplate. For example, the shaft 510 and the actuator 570 together can be moved back and forth along the longitudinal axis of the shaft 510 along the directions of arrows “X” and “Y” as shown in FIG. 26. This movement allows the cutting region 530 to engage and scrape or cut an endplate. In addition, the shaft 510 and the actuator 570 can be rotated along the longitudinal axis of the shaft 510 along the directions of arrows “Z” and “AA” as shown in FIG. 27.

When the process of cutting or scraping the endplates or facet joint articulating surfaces has been completed, the tool 500 can be manipulated to return to its collapsed or delivery configuration. To collapse the cutting region 530, the actuator 570 is moved relative to the shaft 510 along the direction of arrow “V” in FIG. 26. When the actuator 570 moves the distal end 504 of the shaft 510 to its farthest position, the cutting element 540 will be linear and disposed within the cylindrical profile of the shaft 510.

An alternative embodiment of a tool according to the invention is illustrated in FIGS. 29-31. Tool 600 can be inserted through a delivery device and deployed in a disc space to induce the flow of blood into the disc space or facet joint. The tool 600 can be manipulated by a user to engage a superior endplate and/or an inferior endplate in a disc space or articulating surfaces in a facet joint.

In this embodiment, tool 600 includes a shaft 610 with a proximal end 602 and an opposite, distal end 604. Similar to shafts 310, 410, and 510, shaft 610 is a tube with an outer surface 612 and an inner surface 614 that defines a channel 616 extending through the shaft 610. Also, the shaft 610 has a substantially cylindrical cross-sectional configuration.

As illustrated in FIG. 29, the shaft 610 includes a cutting region or portion 630. The cutting region 630 is adjustable and the tool 600 can be manipulated so that the cutting region 630 engages a target region in the disc space.

As illustrated in FIG. 29, an opening or recess 632 is formed in the shaft 610 in the cutting region 630. The opening 632 extends from one side of the shaft 610 to the other side of the shaft 610. The opening 632 can be formed using a material cutting process, such as EDM. The opening 632 is defined by surface 638 and extends between ends 634 and 636. The opening 632 is in communication with the channel 616 of the shaft 610.

In the cutting region 630, cutting elements or members 640A and 640B are formed in the cutting region 630. The cutting region 630 of tool 600 is similar to the cutting region 530 of tool 500 except that the cutting region 630 includes two cutting elements 640A and 640B. As shown, the cutting elements 640A and 640B are located on opposite sides of the shaft 610.

Referring to FIG. 230, after the opening 632 has been made through the shaft 610, an actuator 670 is inserted into the channel 616 of the shaft 610. The actuator 670 has a proximal end 672 and a distal end 674 which is coupled to the shaft 610 proximate to shaft end 604. The proximal end 672 of the actuator 670 is not coupled to the shaft 610 and can be manipulated by a user. In this embodiment, actuator 670 is a substantially cylindrical rod.

The cutting region 630 of the shaft 610 is illustrated in a delivery or unbiased configuration 680 in FIGS. 29 and 30. In this configuration 680, the cutting elements 640A and 640B are stretched out and disposed within the substantially cylindrical profile of the shaft 610. Thus, the cutting elements 640A and 640B do not extend outwardly beyond the original cylindrical shape of the shaft 610.

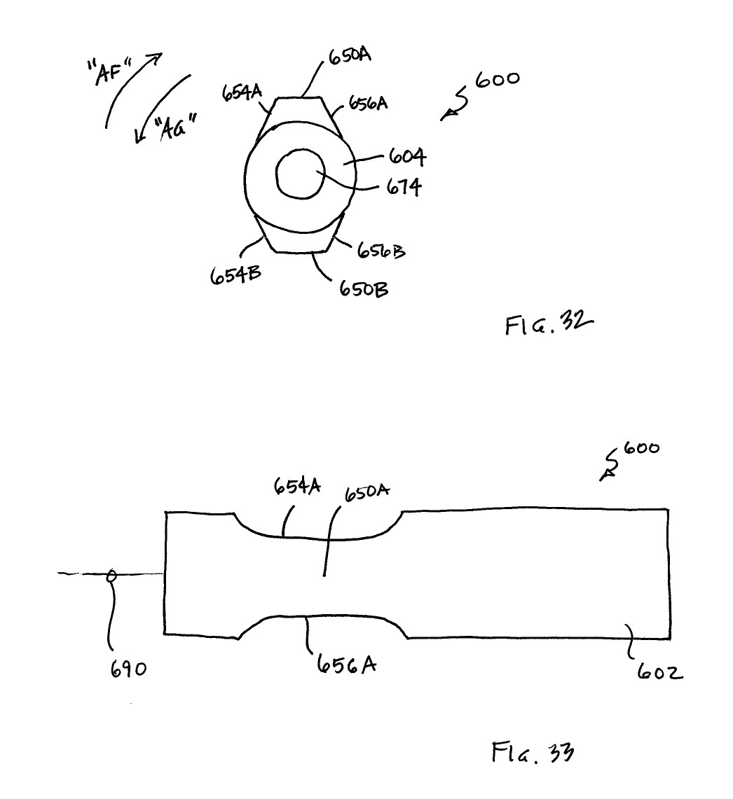

Referring to FIG. 31, an exemplary method of adjusting the tool 600 is illustrated. Adjustment of the tool 600 occurs after the tool 600 has been deployed through a delivery device and the cutting region 630 of the tool 600 is located proximate to the target area in a disc space. The tool 600 can be adjusted so that the cutting region 630 is in an expanded or deployed configuration 682 as illustrated in FIGS. 31-33. FIG. 31 illustrates a side view of the tool 600, FIG. 32 illustrates an end view of the tool 600, and FIG. 33 illustrates a bottom view of the tool 600.

The proximal end 672 of the actuator 670 can be manipulated or moved relative to the shaft 610. The movement of the actuator 670 relative to the shaft 610 causes the distal end 604 of the shaft 610 to move relative to the proximal end 602 of the shaft 610, thereby causing the shape or configuration of the cutting region 630 to change.

The actuator 670 can be moved along the direction of arrow “AB” in FIG. 31 and such movement causes the distal end 604 of the shaft 610 to move in the same direction. As the distal end 604 moves toward the proximal end 602, the cutting elements 640A and 640B expand outwardly as illustrated in FIG. 31 because those parts of the shaft 610 are the weakest portions and the remaining portions in that area. The cutting elements 640A and 640B include first portions 642A and 642B and second portions 646A and 646B, respectively. When the cutting elements 640A and 640B are expanded outwardly, their first portions and second portions can have a curved configuration as shown in this embodiment, or alternatively, can be substantially linear.

When the cutting region 630 is expands, engaging areas 650A and 650B are formed on cutting elements 640A and 640B, respectively. As illustrated in FIG. 32, engaging area 650A includes sides 654A and 656A that have sharp edges that can be used to cut or scrap an endplate. Similarly, engaging area 650B includes sides 654B and 656B that have sharp edges. The distance that the cutting elements 640A and 640B extend outwardly from the shaft 610 is the same and is determined by the distance that the actuator 670 is moved along the direction of arrow “AB” in FIG. 31.

When the cutting region 630 is disposed in its expanded or deployed configuration 682, the tool 600 can be manipulated so that the cutting region 630 engages the target area, such as a superior endplate or an inferior endplate. For example, the shaft 610 and the actuator 670 together can be moved back and forth along the longitudinal axis of the shaft 610 along the directions of arrows “AD” and “AE” as shown in FIG. 31. This movement allows the cutting region 630 to engage and scrape or cut an endplate. In addition, the shaft 610 and the actuator 670 can be rotated along the longitudinal axis of the shaft 610 along the directions of arrows “AF” and “AG” as shown in FIG. 32.

When the process of cutting or scraping the endplates or facet joint articulating surfaces has been completed, the tool 600 can be manipulated to return to its collapsed or delivery configuration. To collapse the cutting region 630, the actuator 670 is moved relative to the shaft 610 along the direction of arrow “AC” in FIG. 31. When the actuator 670 moves the distal end 604 of the shaft 610 to its farthest position, the cutting element 640 will be linear and disposed within the cylindrical profile of the shaft 610.

An alternative embodiment of a tool according to the invention is illustrated in FIGS. 34-36. The structure and use of tool 700 is substantially the same as the structure and use of tool 600, which was previously described. The differences between tool 700 and tool 600 will be identified and described.

Tool 700 includes a shaft 710 with a proximal end 702 and an opposite, distal end 704. Shaft 710 is a tube with an outer surface 712 and an inner surface 714 that defines a channel 716 extending through the shaft 710.

As illustrated in FIG. 34, the shaft 710 includes a cutting region or portion 730. The cutting region 730 is adjustable and the tool 700 can be manipulated so that the cutting region 730 engages a target region in the disc space.

As illustrated in FIG. 34, an opening or recess 732 is formed in the shaft 710 that through the shaft 710. The opening 732 can be formed using a material cutting process, such as EDM. The opening 732 extends from end 734 to end 736 and is in communication with the channel 716 of the shaft 710. In the cutting region 730, cutting elements or members 740A and 740B are formed in the cutting region 730 and are located on opposite sides of the shaft 710.

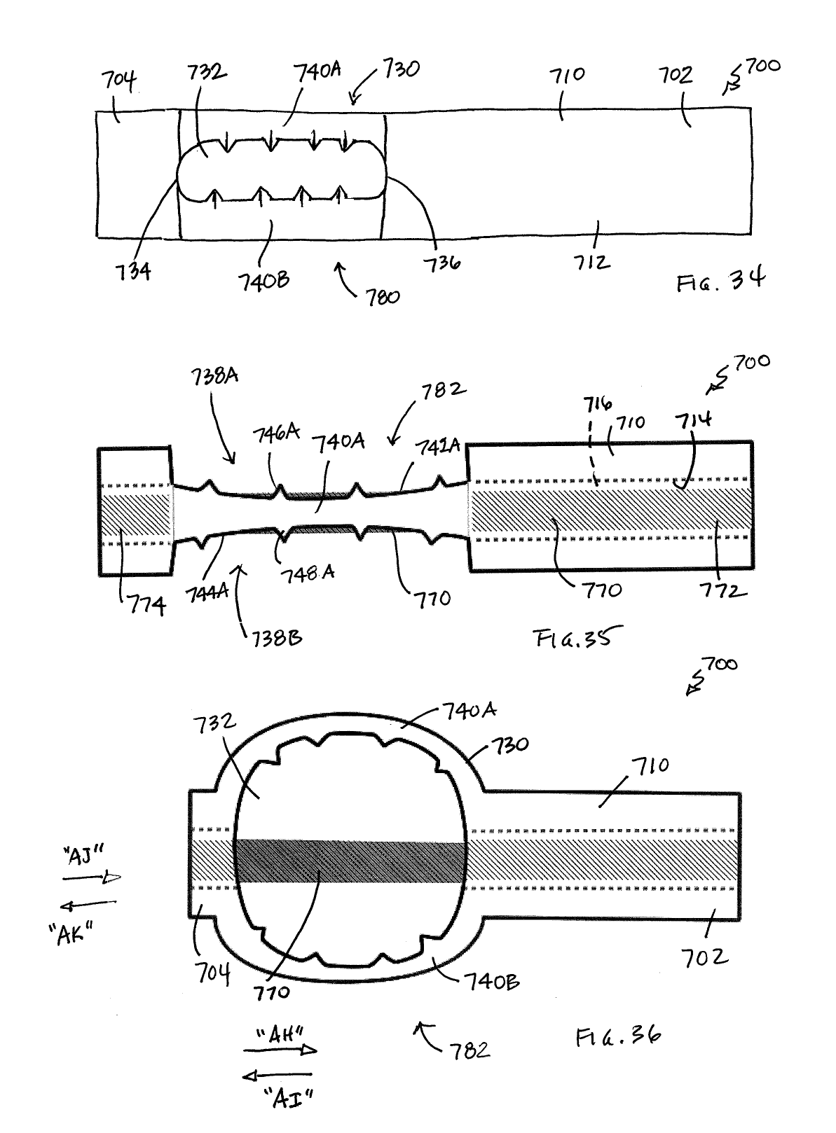

Referring to FIG. 35, after the opening 732 has been made through the shaft 710, an actuator 770 is inserted into the channel 716 of the shaft 710. The actuator 770 has a proximal end 772 and a distal end 774 which is coupled to the shaft 710 proximate to shaft end 704. The proximal end 772 of the actuator 770 is not coupled to the shaft 710 and can be manipulated by a user. Actuator 770 can be similar to any of the actuators described herein.

The cutting region 730 of the shaft 710 is illustrated in a delivery or unbiased configuration 780 in FIGS. 34 and 35. In this configuration 780, the cutting elements 740A and 740B are stretched out and disposed within the substantially cylindrical profile of the shaft 710. Thus, the cutting elements 740A and 740B do not extend outwardly beyond the original cylindrical shape of the shaft 710.

Referring to FIG. 36, an exemplary method of adjusting the tool 700 is illustrated. Adjustment of the tool 700 can be performed in a manner similar to the adjustment of tool 600. Once the tool 700 has been deployed through a delivery device, the tool 700 can be adjusted so that the cutting region 730 is in an expanded or deployed configuration 782 as illustrated in FIG. 36.

The proximal end 772 of the actuator 770 can be manipulated or moved relative to the shaft 710. The movement of the actuator 770 relative to the shaft 710 causes the distal end 704 of the shaft 710 to move relative to the proximal end 702 of the shaft 710, thereby causing the shape or configuration of the cutting region 730 to change.

The actuator 770 can be moved along the direction of arrow “AH” in FIG. 36. As a result, the distal end 704 of the shaft 710 moves in the same direction toward the proximal end 702, and the cutting elements 740A and 740B expand outwardly as illustrated in FIG. 36. When the cutting elements 740A and 740B are expanded outwardly, their first portions and second portions can have a curved configuration as shown in this embodiment, or alternatively, can be substantially linear.

When the cutting region 730 expands, each cutting element 740A and 740B can have an engaging area. Cutting element 740A and 740B can be structured similarly and accordingly, only cutting element 740A will be described for reasons of simplicity only. As illustrated in FIG. 35, cutting element 740A includes sides 742A and 744A that have sharp edges that can be used to cut or scrap an object. In this embodiment, side 742A includes several spaced apart teeth 746A. Similarly, side 744A includes several spaced apart teeth 748A. The teeth 746A and 748A provide increased cutting and scraping functionality for the cutting element 740A because of the sharp points and edges of the teeth 746A and 748A. As shown, the teeth 746A and 748A extend outwardly from the cutting region 730.

When the cutting region 730 is disposed in its expanded or deployed configuration 782, the tool 700 can be manipulated so that the cutting region 730 engages the desired target area. Shaft 710 and actuator 770 can be moved back and forth together along the longitudinal axis of the shaft 710 along the directions of arrows “AJ” and “AK” as shown in FIG. 36. This movement allows the cutting region 730 to engage and scrape or cut an endplate. In addition, the shaft 710 and the actuator 770 can be rotated along the longitudinal axis of the shaft 710.

When the process of cutting or scraping the endplates or facet joint articulating surfaces has been completed, the tool 700 can be manipulated to return to its collapsed or delivery configuration. To collapse the cutting region 730, the actuator 770 is moved relative to the shaft 710 along the direction of arrow “A1” in FIG. 36. When the actuator 770 moves the distal end 704 of the shaft 710 to its farthest position, the cutting element 740 will be linear and disposed within the cylindrical profile of the shaft 710.

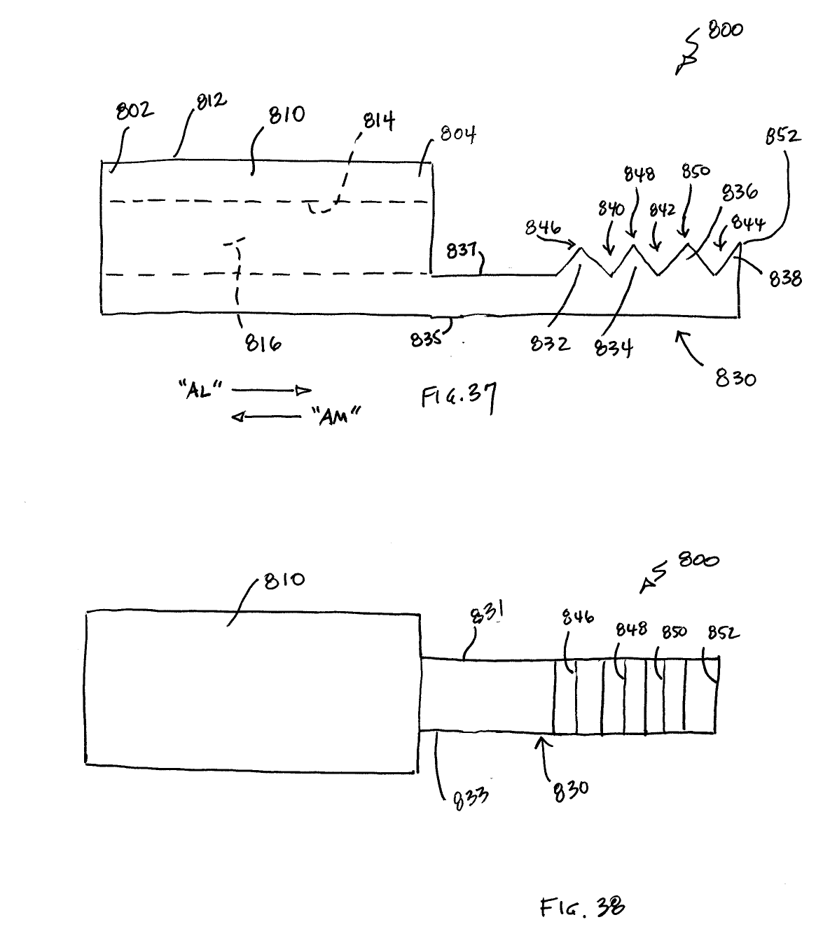

An alternative embodiment of a tool according to the invention is illustrated in FIGS. 37-38. In this embodiment, tool 800 is a single piece or component that can be used in a manner similar to other tools described herein to engage a target area, such as an endplate.

Tool 800 includes a shaft or support portion 810 that has a proximal end 802, a distal end 804, an outer surface 812, and an inner surface 814 defining a channel 816. The channel 816 can be used as a passageway through which debris and materials from the site preparation process can be withdrawn and removed from the disc space. In other embodiments of tools according to the invention, a channel may be formed through which debris and other materials can be suctioned, vacuumed or otherwise removed from the disc space.

Coupled to the shaft 810 is a cutting portion 830 that has sides 831 and 833. In this embodiment, the shaft 810 and the cutting portion 830 are integrally formed and originate as a single piece of material. In other embodiments, the shaft 810 and the cutting portion 830 can be formed as separate components and subsequently coupled together.

Referring to FIG. 37, the cutting portion 830 has an inner surface 837 and an outer surface 835. The cutting portion 830 also includes several cutting members or elements 832, 834, 836, and 838. Between adjacent cutting elements 832, 834, 836, and 838 are recesses 840, 842, and 844. In this embodiment, the cutting portion 830 includes four cutting elements and three recesses. However, in alternative embodiments, the quantity of cutting elements and recesses, as well as the spacing between them, can vary.

Each of the cutting elements 832, 834, 836, and 838 forms a cutting tip 846, 848, 850, and 852, respectively. The cutting tips are surfaces that can be used to cut or scrape a target region.

The tool 800 can be manipulated so that the cutting region 830 engages a target region. When the cutting region 830 is in the desired position, the shaft 810 can be moved back and forth along the direction of arrows “AL” and “AM” so that the cutting region 830 repeated cuts or scrapes the target region.

In an alternative embodiment, the cutting region 830 can be formed so that it extends along a line that is at an angle relative to the longitudinal axis. In particular, the cutting region 830 can be slightly bent outwardly, in which case the teeth of the cutting region 830 are slightly more exposed and configured to engage more of the target region.

An alternative embodiment of a tool according to the invention is illustrated in FIGS. 39-41. In this embodiment, the tool 900 has a proximal end 902 and a distal end 904 and is configured so it can be inserted through a delivery device to a target area. Tool 900 has a substantially cylindrical cross-sectional configuration.

Tool 900 includes a shaft 910 that has a support portion 912 and a cutting portion 914. In this embodiment, the support portion 912 and the cutting portion 914 are integrally formed. In other embodiments, the support portion 912 and the cutting portion 914 are formed separately and subsequently coupled together.

The cutting portion 914 of the tool 900 has multiple configurations. One such configuration is a delivery configuration 916 as illustrated in FIG. 39. In this configuration 916, the shaft portion 912 and the cutting portion 914 are substantially aligned with each other and the longitudinal axis of the shaft 910. When the shaft portion 912 and the cutting portion 914 are aligned, the tool 900 can be inserted and passed through or withdrawn from a delivery device, such as a needle or cannula.

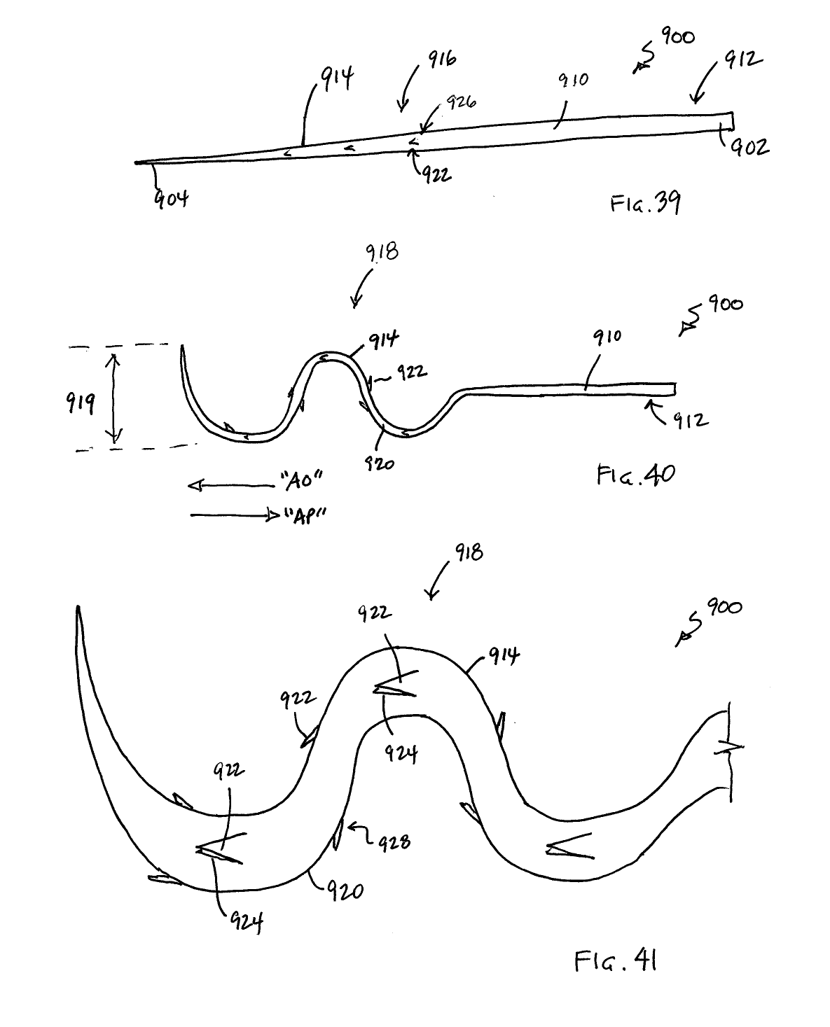

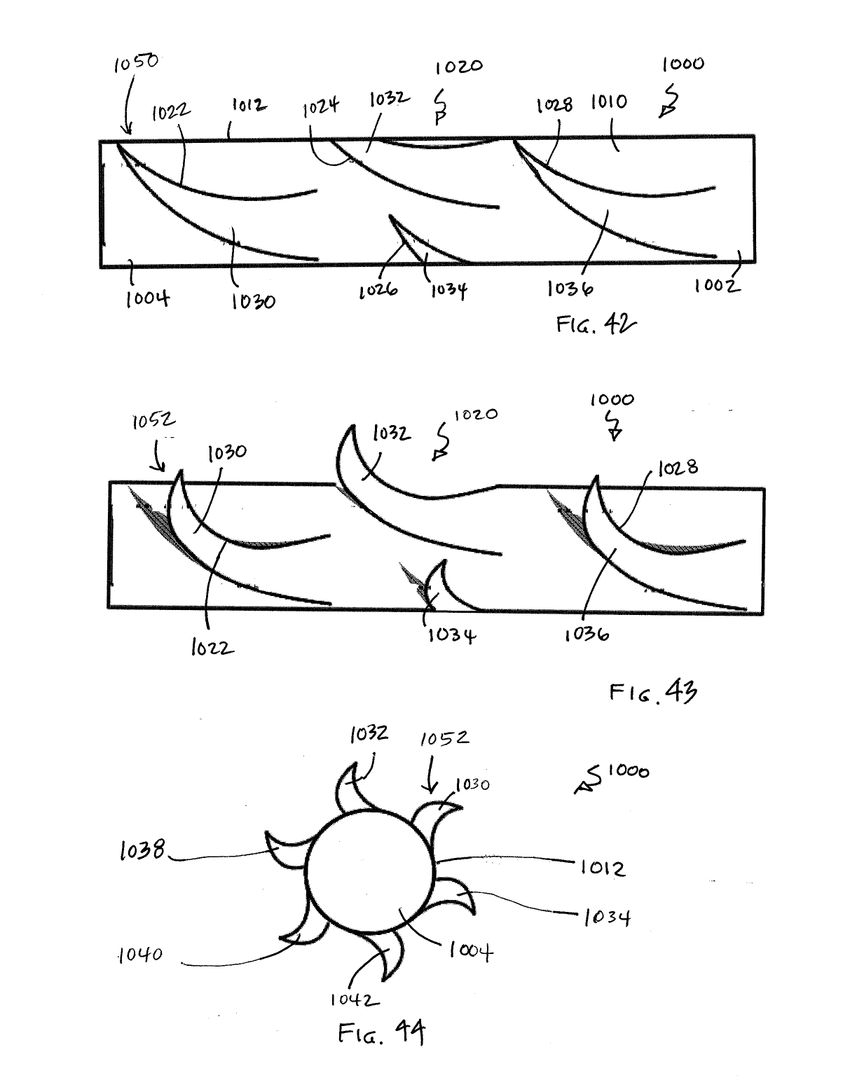

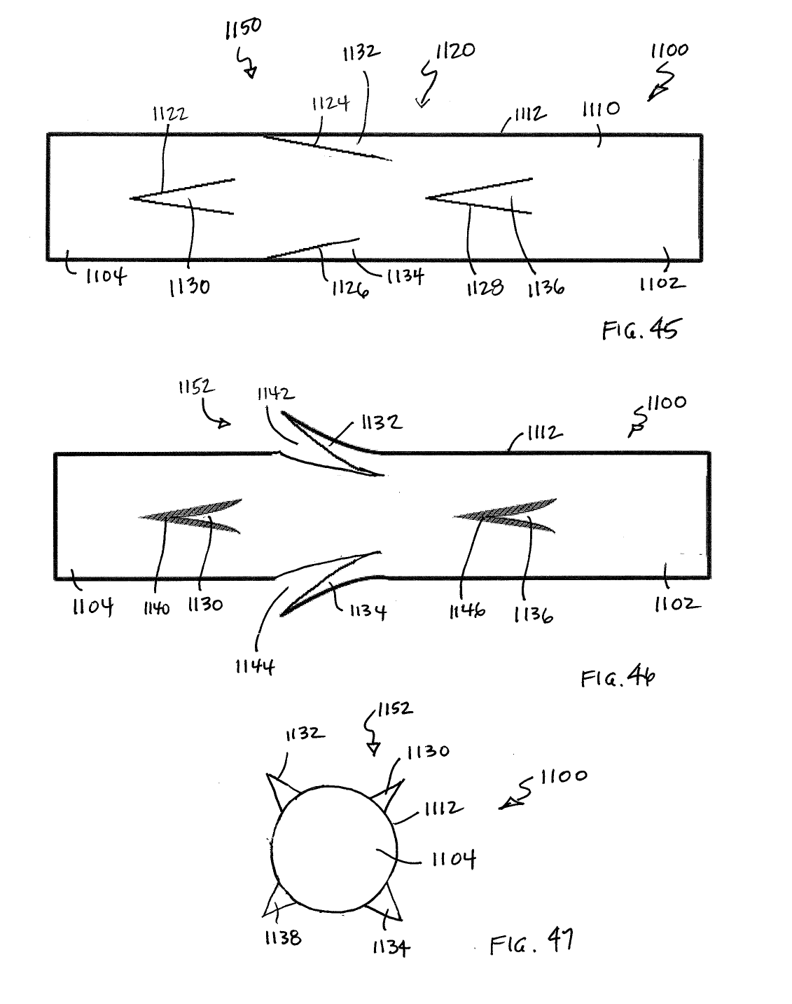

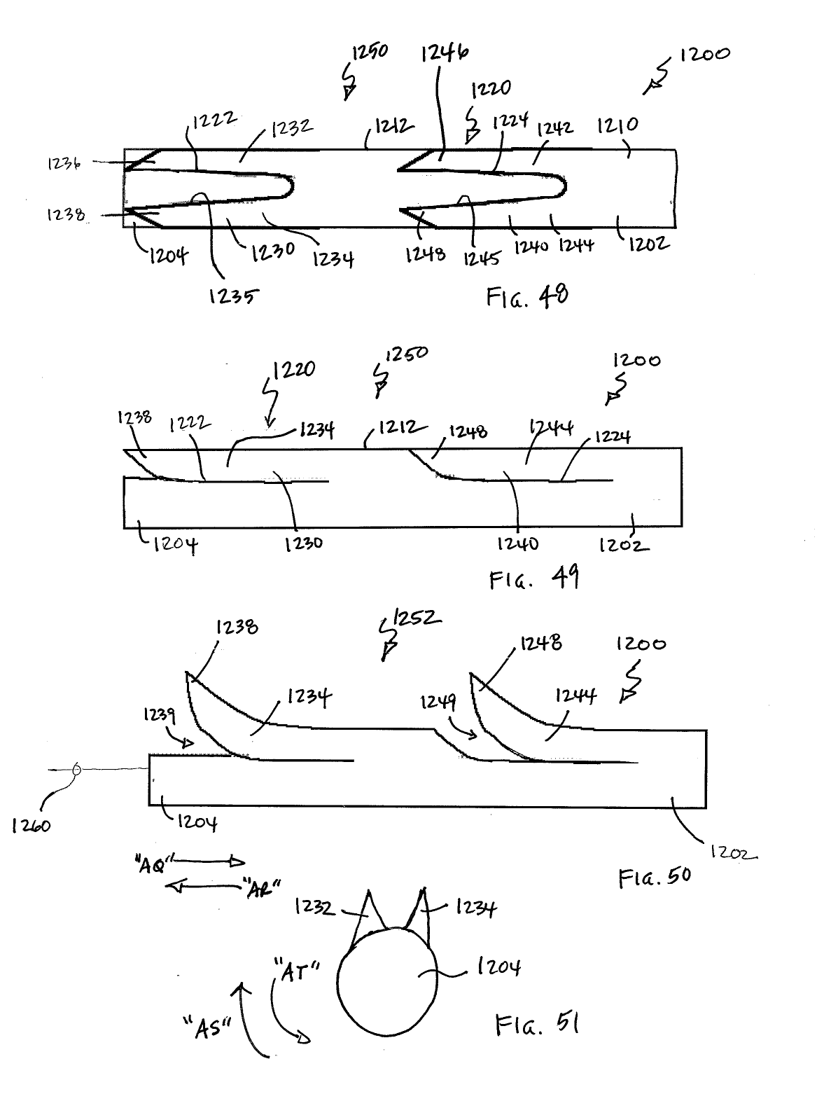

Another configuration is a deployed configuration 918 as illustrated in FIGS. 40 and 41. In this configuration 918, the shape of the cutting portion 914 changes, and the cutting portion 914 is no longer aligned with the support portion 912. As illustrated, the cutting portion 914 has a curved shape and more particularly, has a sinusoidal configuration. In other embodiments, the cutting portion 914 can have a different curved configuration.

By changing the configuration of the cutting portion 914, the surface area that can be prepared by the tool 900 increases. In other words, a much wider cutting or scraping area can be formed (see reference 919 in FIG. 40) when the tool 900 is repeatedly moved back and forth along the directions of arrows “AO” and “AP” as compared to the delivery configuration 916 (see FIG. 39).

In this embodiment, the cutting portion 914 can be formed of a flexible material and has a shape-changing behavior. For example, the flexible material can be as stainless steel, nickel-titanium alloys (NITINOL material), and other metal alloys. Configuration 918 represents an initial or undeformed state of the cutting portion 914.

As the tool 900 is inserted into the delivery device, the cutting portion 914 experiences elastic deformation, such as being spring loaded, and assumes a second, delivery or deformed configuration 916 in which the cutting portion 914 is substantially linear with the support portion 912 and collinear with the longitudinal axis of the shaft 910.

As the cutting portion 914 extends beyond the end of the delivery device, the spring bias arising from elastic deformation tends to move the cutting portion 914 from configuration 916 to configuration 918. The cutting portion 914 seeks to return to configuration 918 because it is an undeformed configuration.

In an alternative embodiment, the cutting portion 914 may be “trained” to change to configuration 918 in the presence of heat of a certain temperature. In this example, the tool 900 is substantially linear and as the cutting portion 914 exits the delivery device and is exposed to the heat of the patient's body, the cutting portion 914 changes to the deployed configuration 918.

As illustrated in FIGS. 39-41, several cutting elements or protrusions 922 are formed in the cutting portion 914 at spaced apart locations. The cutting portion 914 includes an outer surface 920. The cutting elements 922 can have the same properties and behavior as the cutting portion 914. When the cutting portion 914 changes to its deployed configuration 918, the cutting elements 922 extend outwardly from the cutting portion 914. The cutting elements 922 function as cutting or scraping points as the tool 900 is moved along the directions of arrows “AO” and “AP.”

Each cutting element 922 is formed from a portion of the shaft 910 and extends from and is retractable into a notch 924 from which the cutting element 922 was cut. When the cutting portion 914 returns to its delivery configuration 916 (see FIG. 39), each cutting element 922 retracts back into its respective notch 924.

In different embodiments, the size, quantity, and location of the cutting elements formed on the shaft 910 can vary.

An alternative embodiment of a tool according to the invention is illustrated in FIGS. 42-44. In this embodiment, tool 1000 is formed of a shaft 1010 that has a proximal end 1002 and a distal end 1004. The shaft 1010 is substantially cylindrical and has a longitudinal axis and has an outer surface 1012 that extends the length of the shaft 1010.

The shaft 1010 includes a cutting portion 1020 in which several cutting elements are formed. Several slits or cuts 1022, 1024, 1026, and 1028 are made in the outer surface 1012 of the shaft 1010. The cuts 1022, 1024, 1026, and 1028 do not extend through the shaft 1010. Each set of cuts forms a cutting element or member or protrusion. For example, cut 1022 defines cutting element 1030, cut 1024 defines cutting element 1032, cut 1026 defines cutting element 1034, and cut 1028 defines cutting element 1036. While only four sets of cuts and cutting elements are illustrated and described with respect to FIG. 42, any number of cuts and cutting elements can be formed in the shaft 1010 at spaced apart locations.

Similar to many of the tools previously described, tool 1000 has multiple configurations. A delivery or undeformed configuration 1050 is illustrated in FIG. 42. In this configuration 1050, the cutting elements 1030, 1032, 1034, and 1036 do not extend outwardly from the shaft 1010 and are disposed within the substantially cylindrical profile of the shaft 1010.

A deformed or deployed configuration 1052 of the cutting portion 1020 is illustrated in FIGS. 43 and 44. As illustrated, in this configuration 1052, the cutting elements 1030, 1032, 1034, and 1036 are curved and extend outwardly from the shaft 1010. FIG. 44 illustrates an end view of the tool 1000 and additional cutting elements 1038, 1040, and 1042 are shown.

The cutting elements are formed of the same material of the shaft 1010 which has elastic properties. The cutting elements can be “trained” so that upon the presence of heat of a certain temperature or a sufficient amount of heat will cause the cutting elements to move outwardly.

An alternative embodiment of a tool according to the invention is illustrated in FIGS. 45-47. In this embodiment, tool 1100 is formed of a shaft 1110 that has a proximal end 1102 and a distal end 1104. The structure of the shaft 1110 of tool 1100 is substantially similar to the structure of the shaft 1010 of tool 1000 as previously described. In this embodiment, the shaft 1110 is substantially cylindrical, has a longitudinal axis, and an outer surface 1112 that extends the length of the shaft 1110.

The shaft 1110 includes a cutting portion 1120 in which several cutting elements are formed. Several slits or cuts 1122, 1124, 1126, and 1128 are made in the outer surface 1112 of the shaft 1110 and do not extend through the shaft 1110. Each set of cuts forms a cutting element or member. For example, cut 1122 defines cutting element 1130, cut 1124 defines cutting element 1132, cut 1126 defines cutting element 1134, and cut 1128 defines cutting element 1136. While only four sets of cuts and cutting elements are illustrated and described with respect to FIGS. 45 and 46, any number of cuts and cutting elements can be formed in the shaft 1110 at spaced apart locations.

Similar to many of the tools previously described, tool 1100 has multiple configurations. A delivery or undeformed configuration 1150 is illustrated in FIG. 45. In this configuration 1150, the cutting elements 1130, 1132, 1134, and 1136 do not extend outwardly from the shaft 1110 and are disposed within the substantially cylindrical profile of the shaft 1110.

A deformed or deployed configuration 1152 of the cutting portion 1120 is illustrated in FIGS. 46 and 47. As illustrated, in this configuration 1152, the cutting elements 1130, 1132, 1134, and 1136 are curved and extend outwardly from the shaft 1110. FIG. 47 illustrates an end view of the tool 1100 and an additional cutting element 1138 is shown.

The cutting elements are formed of the same material of the shaft 1110 which has elastic properties. The cutting elements can be “trained” so that upon the presence of heat of a certain temperature or a sufficient amount of heat will cause the cutting elements to move outwardly. As the tool 1110 is withdrawn into the delivery device, the cutting elements are pushed inwardly toward the body of the shaft 1110. In particular, cutting element 1130 moves into opening or recess 1140, cutting element 1132 moves into opening 1142, cutting element 1134 moves into opening 1144, and cutting element 1136 moves into opening 1146.

Another embodiment of a tool according to the invention is illustrated in FIGS. 48-51. In this embodiment, tool 1200 includes a shaft 1210 that has a proximal end 1202 and a distal end 1204. The shaft 1210 has an outer surface 1212 that extends along its length and a longitudinal axis 1260.

The shaft 1210 includes a cutting portion 1220 that can be disposed in multiple configurations. A delivery configuration 1250 is illustrated in FIGS. 48 and 49 and a deployed configuration 1252 is illustrated in FIGS. 50 and 51. These configurations 1250 and 1252 are similar to the corresponding configurations of the previously described tools 1000 and 1100.

As illustrated in FIGS. 48 and 49, cutting members or elements 1230 and 1240 have different shapes and configurations than the previously described cutting elements for tools 1000 and 1100. Cutting element 1230 is formed by slit or cut 1222 that extends around a portion of the perimeter of the shaft 1210. The extent and path of the cut 1222 creates the particular shape or configuration of the cutting element 1230. As shown, cutting element 1230 includes two cutting portions 1232 and 1234 that includes inner edges 1235 and tips 1236 and 1238.

Similarly, cutting element 1240 is formed by slit or cut 1224 that extends around a portion of the perimeter of the shaft 1210. The extent and path of the cut 1224 creates the particular shape or configuration of the cutting element 1240. Cutting element 1240 includes two cutting portions 1242 and 1244 that includes inner edges 1245 and tips 1246 and 1248.

Referring to FIGS. 48 and 49, the cutting elements 1230 and 1240 are in their delivery or biased positions. As the cutting portion 1220 exits the delivery device, the resilient nature of cutting element 1230 causes it to curve or flare outwardly as illustrated in FIG. 50. A space or region 1239 is formed beneath the cutting portion 1234 as the cutting portion 1234 moves to its extended or deployed configuration.

As the cutting portion 1220 continues to exit the delivery device, the resilient nature of cutting element 1240 causes it to curve or flare outwardly as illustrated in FIG. 50. A space or region 1249 is formed beneath the cutting portion 1244 as the cutting portion 1244 moves to its extended or deployed configuration.

In one embodiment, each of the cutting elements may be trained to be in its extended position or configuration when no force is applied to the cutting element. In this implementation, a force must be applied to each cutting element so that it moves from its unbiased position to its retracted position. Alternatively, the cutting elements may be formed of a material that can change shape upon the application of heat. In this implementation, the delivery positions 1250 of the cutting elements may be their unbiased positions and when heat is applied to the cutting portion inside the patient's body, the cutting elements can expand or extend outwardly to their deployed positions 1252.

In use, the tool 1200 can be manipulated so that the cutting portion 1220 is repeatedly moved along the direction of arrows “AQ” and “AR.” In addition to that movement, the cutting portion 1220 can be rotated about its longitudinal axis 1260 along the directions of arrows “AS” and “AT.” When the use of the tool 1200 is complete, the tool 1200 can be withdrawn through the delivery device and removed from the patient's body.

An alternative embodiment of a tool according to the invention is illustrated in FIGS. 52-55. In this embodiment, the tool 1300 includes a shaft 1310 having a proximal end 1302 and a distal end 1304. The shaft 1310 includes an outer surface 1312 that extends along its length and a longitudinal axis 1340.

In this embodiment, the shaft 1310 includes multiple portions 1314 and 1316 that are integrally formed. Portion 1314 may have a diameter that is slightly less than the diameter of portion 1316. The smaller diameter increases the flexibility of shaft portion 1314. In other embodiments, the portions 1314 and 1316 may be separately formed and subsequently coupled together. Portion 1314 includes a tip 1315 at its distal end.

The shaft 1310 also includes a cutting portion 1320. Referring to FIG. 53, the cutting portion 1320 includes an area or region 1324 that is formed by removing a portion of the shaft 1310. Surfaces 1322 and 1323 define the area 1324, which is bounded by a curved surface 1332 that terminates in a point or tip 1331. The removal of material decreases the thickness of part of the cutting portion 1320, thereby increasing the flexibility of the cutting portion 1320. The removal of material also makes the tip 1331 more pronounced and facilitates the engagement of the tip 1331 with the desired target area, such as an endplate.

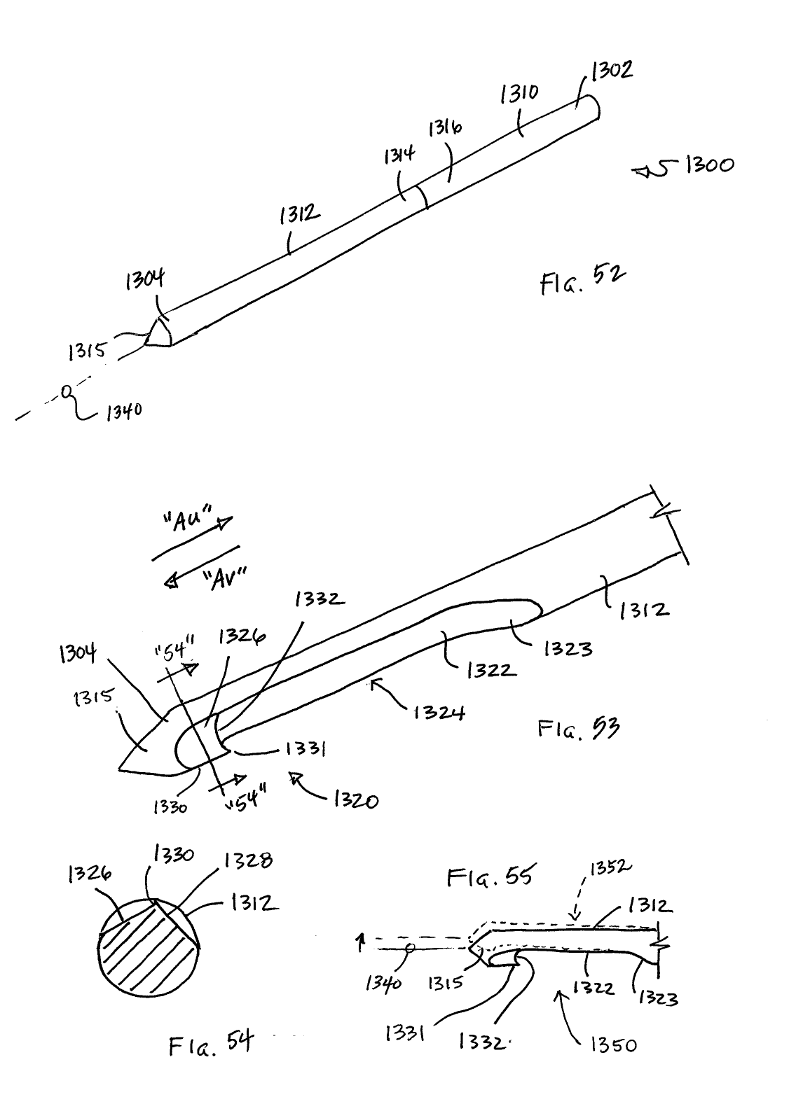

Referring to FIG. 54, a cross-sectional view of a portion of the cutting portion 1320 is illustrated. As shown, sloped surfaces 1326 and 1328 are formed in the shaft 1310 and a ridge or edge 1330 is formed between them. The edge 1330 and the tip 1331 are sharp surfaces that can be used to engage an endplate when the tool 1300 is moved along the directions of arrows “AU” and “AV.”

Referring to FIG. 55, the cutting portion 1320 can be aligned with the longitudinal axis 1340 which extends along the shaft 1310. Cutting portion 1320 is illustrated in that aligned configuration 1350 in solid lines in FIG. 55. In an alternative embodiment, the cutting portion 1320 can be formed so that it is offset from and extends at an angle relative to the longitudinal axis 1340. This offset position 1352 is illustrated in dashed lines in FIG. 55. The offset configuration allows the cutting portion to be more open to the desired target area and accordingly, engage more of the surface area.

An alternative embodiment of a tool according to the invention is illustrated in FIGS. 56-58. Tool 1400 includes a shaft 1410 that has a proximal end 1402 and a distal end 1404. In this embodiment, shaft 1410 includes two portions 1414 and 1416 that have different diameters. Formed as part of the narrower diameter portion 1414 is a cutting portion 1420. Generally, tool 1400 is similar to tool 1300 with the exception that it has two cutting elements at the distal end of the shaft 1400.

Referring to FIGS. 57 and 58, the details of the cutting portion 1420 are illustrated. Cutting portion 1420 includes two cutting elements 1430 and 1450 that are slightly biased apart from each other. When the cutting elements 1430 and 1450 are spread apart, a gap 1426 is formed between them. The length of the gap 1426 from the end of the tool to the end 1425 of the gap 1426 can vary. The longer that the gap 1426 is results in an increase in the distance that the cutting elements 1430 and 1450 are spread apart.

As the tool 1400 is inserted into a delivery device, the cutting elements 1430 and 1450 are forced toward each other. When the cutting portion 1420 extends beyond the distal end of the delivery device, the cutting elements 1430 and 1450 are permitted to spread apart to their unbiased positions.

Referring to FIG. 57, the cutting element 1430 includes a body 1432 that ends in a point 1434. A sloped surface 1440 is formed on a side of the cutting element 1430 and the cutting element 1430 includes a tip 1436. The body 1432 includes a recessed area or region that is defined by a curved surface 1424 at one end and a curved surface 1438 at the other end. The recessed area or region narrows the thickness of the cutting elements and accordingly, increases the flexibility of the cutting elements and the ability of the tip 1436 to engage the targeted endplate. In addition, curved surface 1438 increases the cutting and scraping functionality of the point 1436.

Cutting element 1450 is configured to be a mirror-image of cutting element 1430. As shown, cutting element 1450 includes a body 1452 that ends in a point 1454. A sloped surface 1460 is formed on a side of the cutting element 1450 and forms part of tip 1456. The body 1452 includes a recessed area or region that is defined by a curved surface 1444 at one end and a curved surface 1458 at the other end. The curved surface 1458 increases the cutting and scraping functionality of the tip 1456.

The cutting elements 1430 and 1450 include inner surfaces 1442 and 1462 that are disposed proximate to each other when the cutting elements 1430 and 1450 are moved together. In one implementation, the manner in which tool 1400 can be made is to form tool 1400 to resemble tool 1300 and then cut the cutting portion 1420 in half, thereby forming slit 1426 and cutting elements 1430 and 1450.

Referring to FIG. 58, the cutting portion 1420 of the tool 1400 can be formed to be offset from the longitudinal axis 1470 of the shaft 1410. In one embodiment, the cutting portion 1420 can be formed so that it is aligned with the longitudinal axis 1470. Cutting portion 1420 is illustrated in this alignment position 1480 in solid lines in FIG. 58. In another embodiment, the cutting portion 1420 can be formed so that the cutting elements are extend away from and are offset from the longitudinal axis 1470. This configuration is illustrated as reference 1482 in dashed lines in FIG. 58. When the cutting portion 1420 is offset from the longitudinal axis 1470 in configuration 1482, the tips 1436 and 1456 are able to engage the target region, such as an endplate, easier. The tool 1400 can be moved along the direction of arrows “AW” and “AX” as shown in FIG. 58.

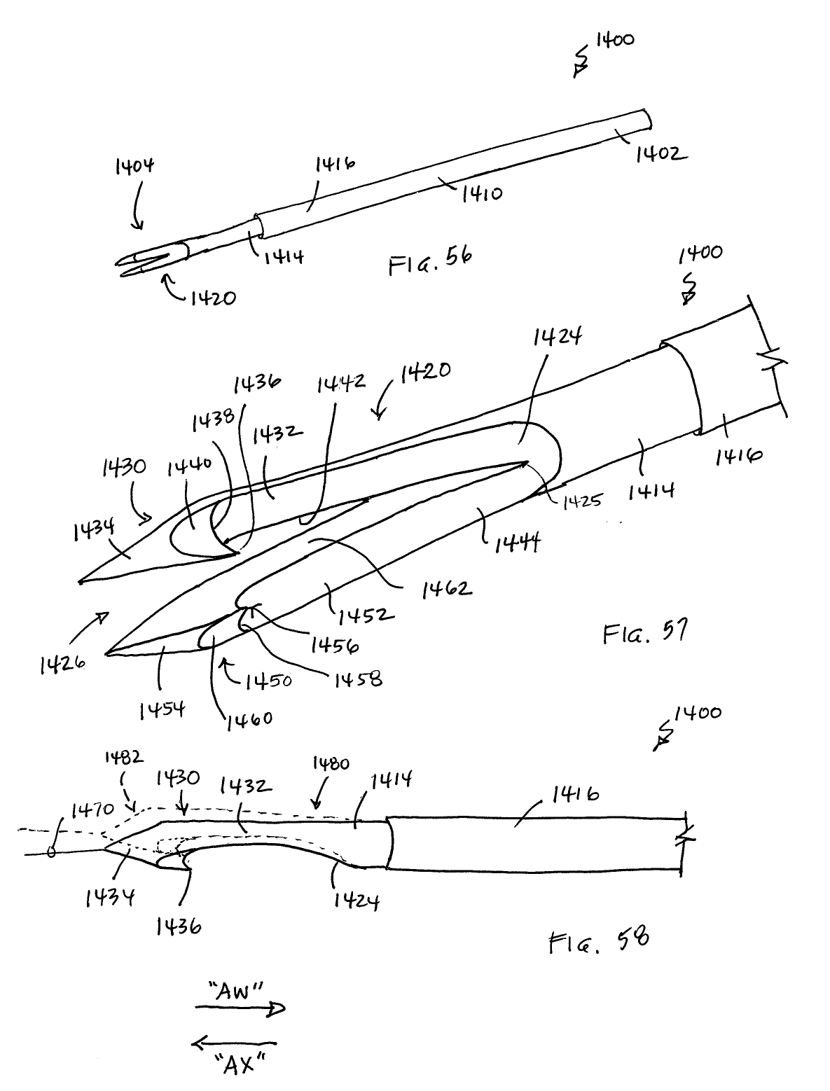

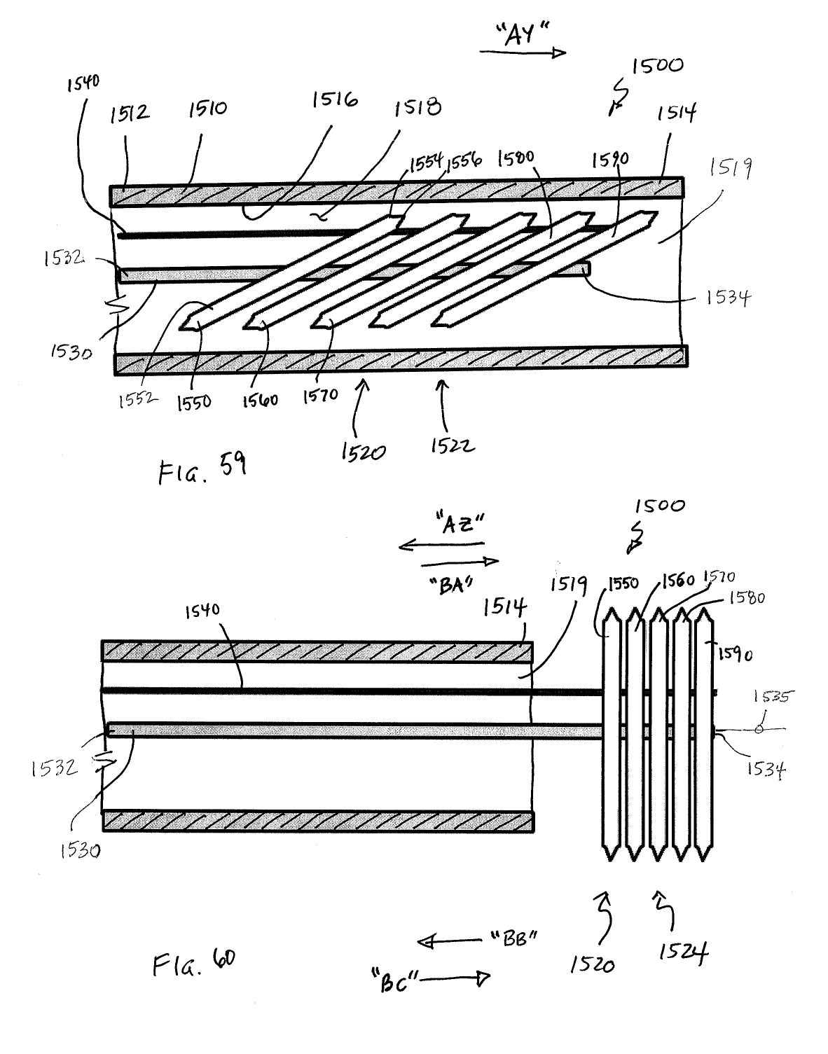

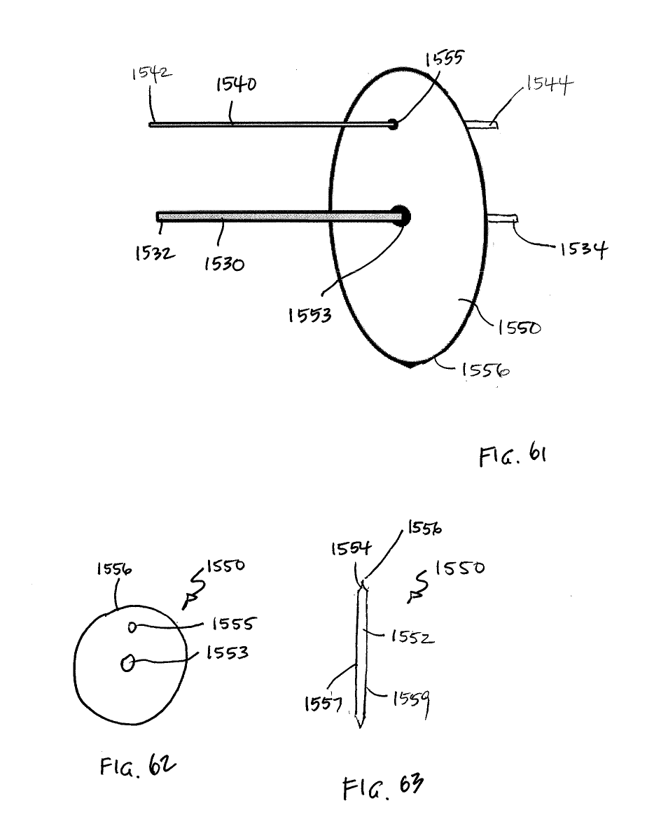

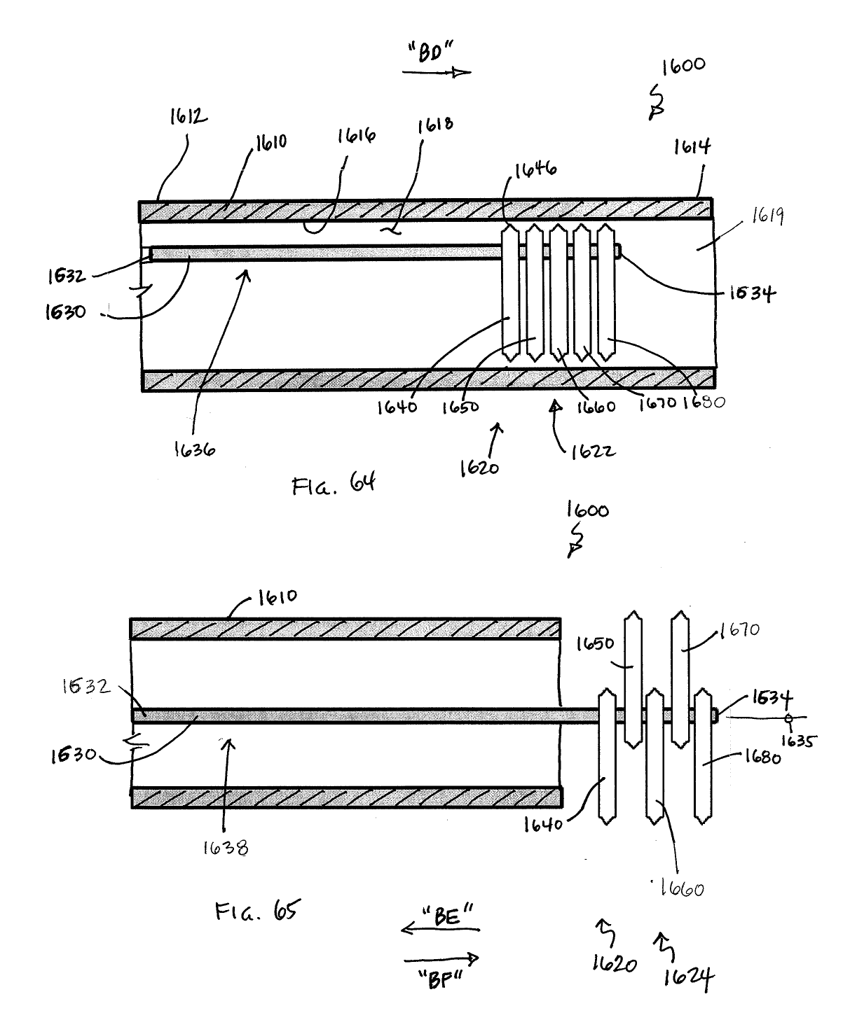

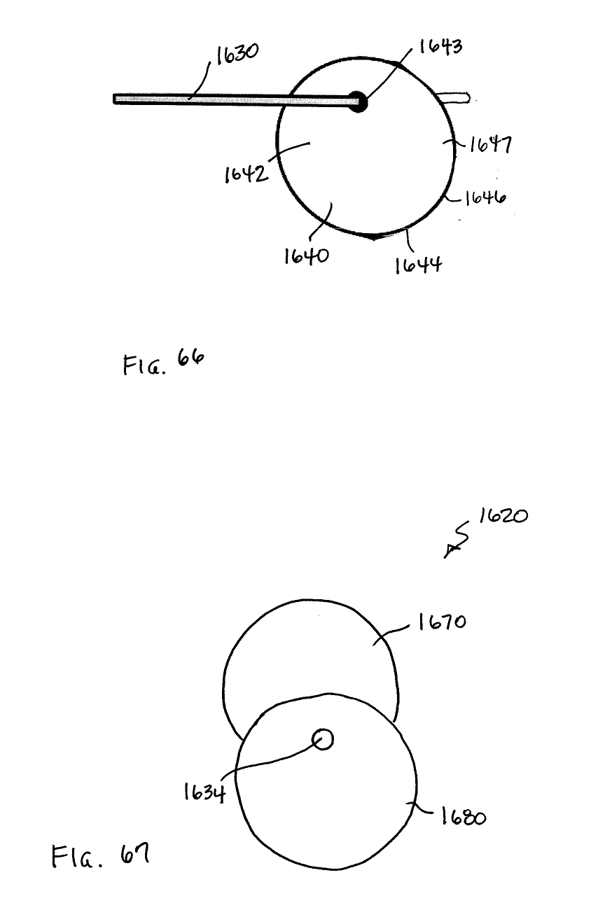

An alternative embodiment of a tool according to the invention is illustrated in FIGS. 59-63. In this embodiment, the tool 1500 includes a preparation device 1520 that can be inserted into and passed through a delivery device 1510.

The delivery device 1510 is exemplary of various delivery devices that can be used with any of the tools disclosed herein. Delivery device 1510 includes a proximal end 1512 and a distal end 1514. An inner surface 1516 extends between the ends 1512 and 1514 and defines a channel 1518 that has an opening 1519 proximate to distal end 1514.

Preparation device 1520 includes a support or rod 1530 with opposite ends 1532 and 1534 and a longitudinal axis 1535. Several cutting elements are movably mounted on the rod 1530. In particular, cutting elements 1550, 1560, 1570, 1580, and 1590 are illustrated as being mounted on the rod 1530. The cutting elements 1550, 1560, 1570, 1580, and 1590 are sufficiently coupled to the rod 1530 so that the cutting elements move with the rod 1530 as the rod 1530 moves along the directions of arrows “AZ” and “BA” (see FIG. 60).

The preparation device 1520 includes an actuator or control rod 1540 with ends 1542 and 1542. Each of the cutting elements 1550, 1560, 1570, 1580, and 1590 is operatively coupled to the actuator 1540 as well. The actuator 1540 can be manipulated to change the configuration of the preparation device 1520. The preparation device 1520, and in particular, the cutting elements 1550, 1560, 1570, 1580, and 1590, can be disposed in multiple positions or configurations. The cutting elements can be disposed in a delivery or collapsed configuration 1522 as illustrated in FIG. 59 and in a deployed or expanded configuration 1524 as illustrated in FIG. 60.

As the cutting elements and the rod 1530 pass through the delivery device 1510, the cutting elements 1550, 1560, 1570, 1580, and 1590 are in their delivery configurations to allow them to pass through the delivery device 1510 which has a smaller dimension than the dimension of the cutting elements. After the cutting elements 1550, 1560, 1570, 1580, and 1590 have passed through opening 1519 of the delivery device 1510, the actuator 1540 can be pulled along the direction of arrow “AZ” with respect to the rod 1530. The relative movement between the actuator 1540 and the rod 1530 causes the cutting elements 1550, 1560, 1570, 1580, and 1590 to pivot about their mountings on the rod 1530 and move to their expanded positions as shown in FIG. 60. At this point, the rod 1530 can be moved along the directions of arrows “BB” and “BC” to engage the target area, such as an end plate. When the site preparation process has been completed, the actuator 1540 is moved along the direction of arrow “BA” and the cutting elements 1550, 1560, 1570, 1580, and 1590 move to their collapsed or delivery configurations.

Referring to FIGS. 61-63, an exemplary embodiment of a cutting element for use with tool 1500 is illustrated. Cutting element 1550 has a substantially circular configuration and resembles a disc. In this embodiment, the cutting elements of tool 1500 have similar configurations and accordingly, only cutting element 1550 is described.

The cutting element 1550 includes a body 1552 with a perimeter portion 1554 that includes a sharp edge 1556. The body 1552 has opposite sides 1557 and 1559 and two holes 1553 and 1555 that extend between the sides 1557 and 1559. Hole 1553 is dimensioned to receive the support rod 1532 and hole 1555 is dimensioned to receive the actuator 1540.

An insert (not shown) can be disposed in each of the holes 1553 and 1555 to operatively couple the cutting element 1550 to the rod 1532 and the actuator 1540 and prevent the cutting element 1550 from sliding along either the rod 1532 and the actuator 1540. In one embodiment, the insert is formed of a rubber-like or elastomeric material and can be coupled to the rod 1532 and actuator 1540. Alternatively, the insert can be inserted and mounted within the holes 1553 and 1555. The insert can have a washer-like configuration with a central opening through which the rod 1532 or the actuator 1540 can pass. The insert is preferably resilient enough to allow the cutting element to move angularly relative to the rod 1532 or actuator 1540, but otherwise retain the cutting element in its position on the rod 1532 or actuator 1540.

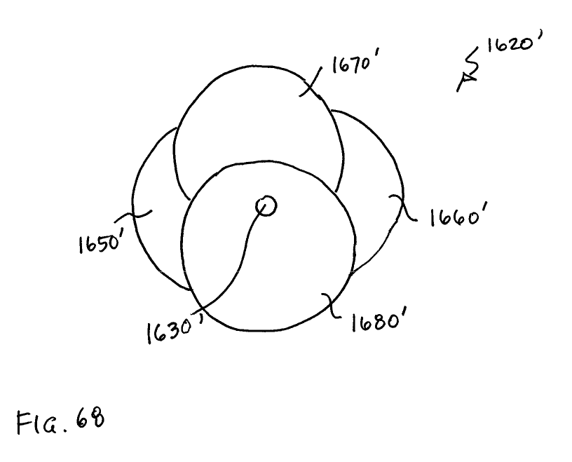

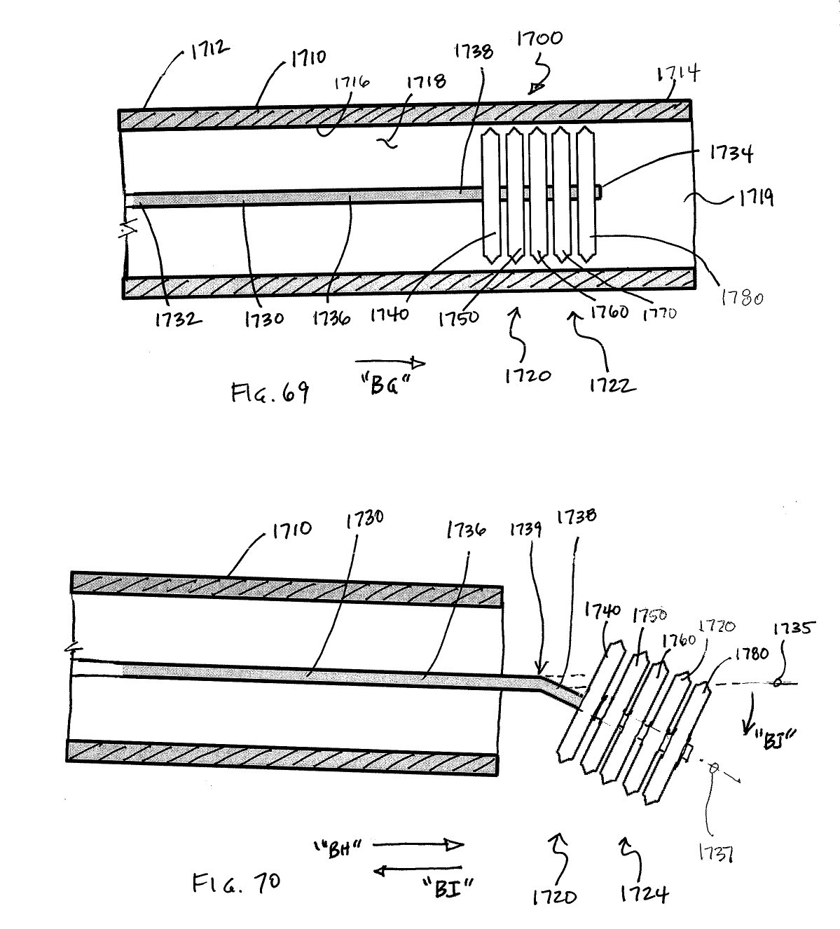

Another embodiment of a tool according to the invention is illustrated in FIGS. 64-68. In this embodiment, the tool 1600 includes a preparation device 1620 that can be inserted and passed through a delivery device 1610. Delivery device 1610 is a tube that has a proximal end 1612, a distal end 1614, and an inner surface 1616 that defines a channel 1618 with an opening 1619.

The preparation device 1620 includes a support or rod 1630 that has a proximal end 1632, a distal end 1634, and a longitudinal axis 1635. As illustrated in FIG. 64, the rod 1630 has several cutting elements mounted on it. Unlike the cutting elements of tool 1500 which were centrally located on rod 1532, the cutting elements 1640, 1650, 1660, 1670, and 1680 of tool 1600 are not mounted on rod 1632 at their centers. In this embodiment, the cutting elements 1640, 1650, 1660, 1670, and 1680 are dimensioned so they can be moved along the channel 1618 of the delivery device without the need to be inclined like the cutting elements of tool 1500.

Referring to FIG. 64, the cutting elements 1640, 1650, 1660, 1670, and 1680 are aligned with each other. In this arrangement, the rod 1630 can be moved toward one side of the channel 1618 and back and forth along the delivery device 1610. The rod 1630 is shown in an offset position 1636. The positions of the cutting elements 1640, 1650, 1660, 1670, and 1680 illustrated in FIG. 64 can be referred to as their delivery positions. In this configuration 1622, the preparation device 1620 can be moved along the direction of arrow “BD” in FIG. 64.

Once the cutting elements 1640, 1650, 1660, 1670, and 1680 pass through opening 1619 of the delivery device 1610, the preparation device 1620 can be adjusted to its deployed configuration 1624 that is illustrated in FIG. 65. The rod 1630 is illustrated in a centrally disposed position 1638. In this embodiment, some of the cutting elements are movably mounted on the rod 1630 and adjustable to positions other than their delivery positions. As illustrated in FIG. 65, cutting element 1650 and cutting element 1670 can each be rotated 180 degrees about its mounting point on rod 1630 to a position that is directly opposite its delivery position. The offset arrangement of the cutting elements 1640, 1650, 1660, 1670, and 1680 forms a preparation tool configuration 1624 that has a greater dimension than the delivery device 1610 diameter.

Referring to FIG. 67, the offset arrangement of cutting elements 1670 and 1680 is illustrated. The increased size of the preparation tool 1620 in this configuration 1624 engages more area on the endplate with each stroke of the preparation tool 1620. When the preparation tool 1620 is in this configuration 1624, the support rod 1630 can be moved along the directions of arrows “BE” and “BF” (see FIG. 65).

When the process of engaging an endplate is completed, the cutting elements 1650 and 1670 are moved to their delivery positions illustrated in FIG. 64. The movement of cutting elements to their delivery positions can be achieved in a variety of manners.

In one embodiment, an internal mechanism can be provided to facilitate the adjustment of one or more of the cutting elements between its delivery position and its deployed configuration. The mechanism may include a pull cord that passes through the support rod 1630 that can be manipulated by a user to cause a cutting element to rotate between its positions.

In another embodiment, the rotational movement of one or more of the cutting elements can be achieved by the rotation of the support rod 1630 along its longitudinal axis 1635. In this case, one or more of the cutting elements is connected to the rod 1630 through a geared relationship. As the rod 1630 is rotated in one direction, the cutting elements that are movably coupled to the rod 1630 move from their delivery configurations to their deployed configurations. The other cutting elements that are fixedly coupled to the rod 1630 do not rotate relative to the rod 1630 and rotate with the rod 1630. To align the cutting elements in this example, the rod 1630 is rotated in an opposite direction until the cutting elements are aligned as illustrated in FIG. 64.

Referring to FIG. 66, an exemplary cutting element is illustrated. Each of the cutting elements 1640, 1650, 1660, 1670, and 1680 has a similar structure, and accordingly, only cutting element 1640 is described in this section for simplicity reasons only. Cutting element 1640 includes a body 1642 with a perimeter portion 1644 with an edge 1646. The body 1642 includes opposite sides 1647 and a hole 1643 extending between sides 1647 through which the rod 1630 is inserted.

In alternative embodiments, various combinations of the cutting elements can be movably mounted on the rod and are rotatable about the rod through different angles. For example, cutting elements can be rotatable about the rod an amount other than 180 degrees.

Referring to FIG. 68, an alternative embodiment of cutting elements that can be used as part of tool 1600 is illustrated. In this embodiment, several cutting elements of a site preparation tool 1620′ are mounted on rod 1630′. Cutting elements 1650′, 1660′, 1670′ and 1680′ are illustrated as being disposed at different positions relative to the rod 1630′. In particular, each of the cutting elements 1650′, 1660′, 1670′, and 1680′ is offset from the other cutting elements by 90 degrees. The profile of the cutting elements 1650′, 1660′, 1670′ and 1680′ has a different configuration than the profile of cutting elements 1640, 1650, 1660, 1670, and 1680 in their deployed positions (see FIG. 67).

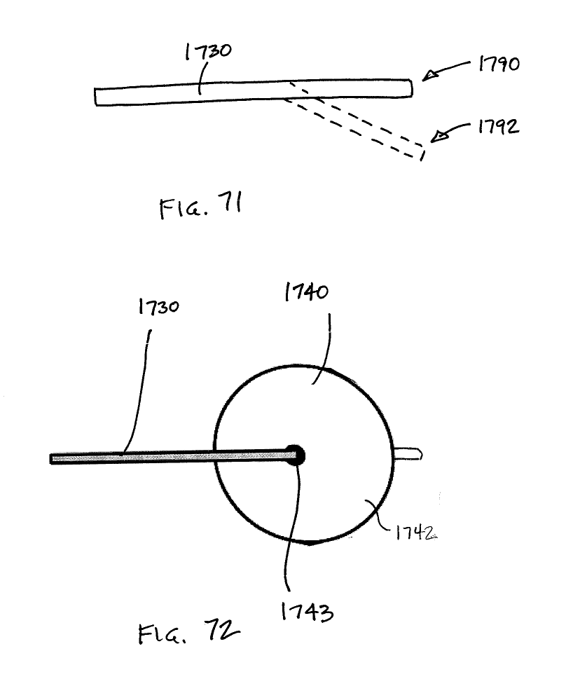

An alternative embodiment of a tool according to the invention is illustrated in FIGS. 69-72. In this embodiment, tool 1700 includes a preparation tool 1720 that can be moved through a delivery device 1710. In this embodiment, delivery device 1710 is a tube that is structurally similar to delivery devices 1510 and 1610 that were previously described. Delivery device 1710 includes a proximal end 1712, a distal end 1714, an inner surface 1716 defining a channel 1718, and an opening 1719.

As illustrated in FIG. 69, preparation tool 1720 has a rod 1730 with a proximal end 1732 and a distal end 1734. Several cutting elements 1740, 1750, 1760, 1770, and 1780 are mounted on rod 1730. In this implementation, the cutting elements are fixedly mounted on the rod 1730. Cutting elements 1740, 1750, 1760, 1770, and 1780 are dimensioned and configured to be deliverable through the delivery device 1710 without adjusting them relative to the rod 1730. In FIG. 69, a delivery configuration 1722 of the preparation tool 1720 is illustrated.

Rod 1730 and cutting elements 1740, 1750, 1760, 1770, and 1780 can be moved along the direction of arrow “BG” (see FIG. 69) through the delivery device 1710. After the cutting elements 1740, 1750, 1760, 1770, and 1780 pass through the opening 1719, the preparation device 1720 changes to its deployed configuration 1724.

In this configuration 1724, a portion of the rod 1730 flexes and changes its shape. The rod 1730 includes a base portion 1736 and a moving portion 1738 that is configured to move relative to the base portion 1736. A bending point 1739 is formed between the base portion 1736 and the moving portion 1738 when the moving portion 1738 adjusts its shape. In one embodiment, the moving portion 1738 of the rod 1730 can be “trained” so that when heat energy is applied to the moving portion 1738, the moving portion 1738 changes from being co-linear with the base portion 1736 to the deployed position illustrated in FIG. 70. In the deployed position, the longitudinal axis 1737 of the moving portion 1738 is offset from longitudinal axis 1735 of the rod 1730. The preparation tool 1720 can be moved along the directions of arrows “BH” and “BI” in FIG. 70 to engage an endplate.

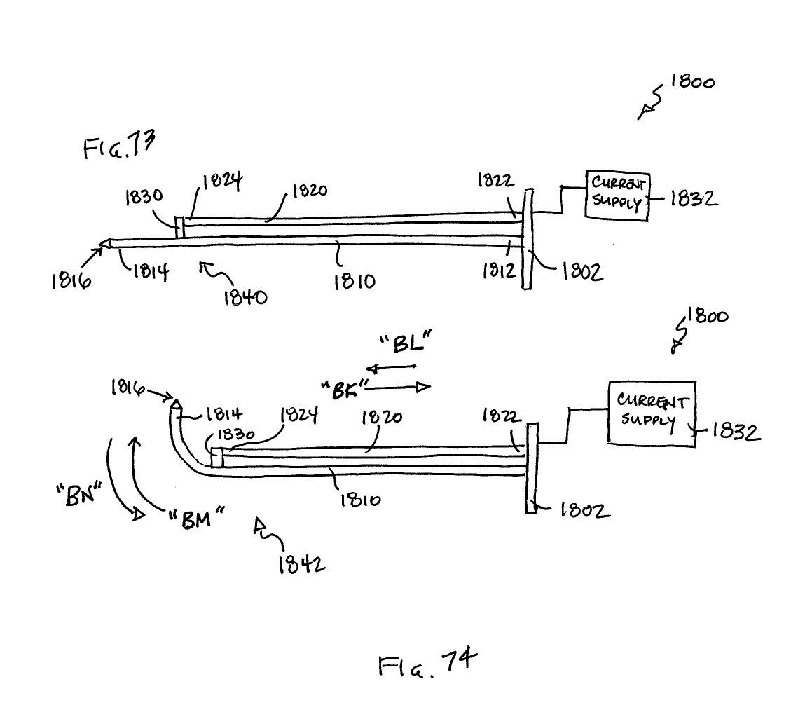

As shown in FIG. 71, the rod 1730 has a substantially linear configuration 1790 and a bent or offset configuration 1792. In alternative embodiments, both the length of the moving portion 1738 and the extent to which it is offset from the longitudinal axis of the rod 1730 can vary. In addition, the quantity and size of the cutting elements can vary as well.

An exemplary embodiment of a cutting element is illustrated in FIG. 72. As illustrated, cutting element 1740 includes a body 1742 and a centrally located hole 1743 that is configured to receive rod 1730.

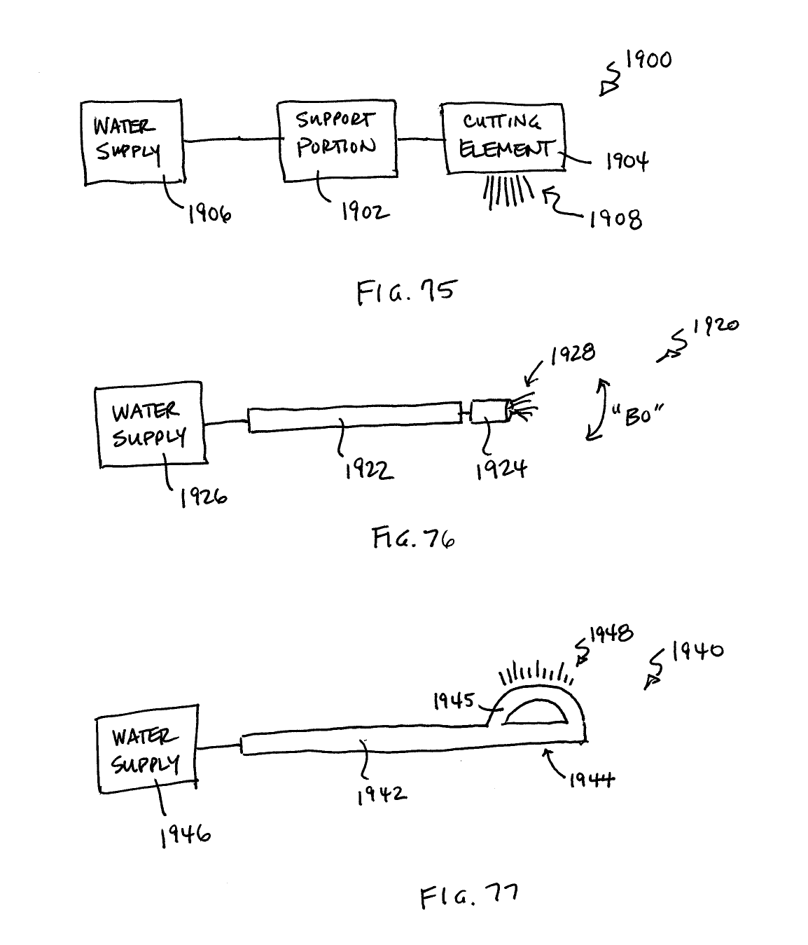

An alternative embodiment of a tool according to the invention is illustrated in FIGS. 73-74. In this embodiment, tool 1800 includes a base 1802, a site preparation element 1810 and a movement element 1820 that is coupled to the site preparation element 1810. As shown, preparation element 1810 has a proximal end 1812 and a distal end 1814. A cutting element 1816, such as an abrasive coated tip, is coupled to or integrally formed at the distal end 1814 of the preparation element 1810.

Coupled to the preparation element 1810 is a movement element 1820 that has a proximal end 1822 and distal end 1824. The movement element 1820 is connected to a coupler 1830 that is attached to the preparation element 1810. A current supply 1832 is connected to the movement element 1820, which is made of a material such as FLEXINOL, which experiences a change in size (such as length) when a current is applied to the material.

A rest or inactive configuration 1840 of the tool 1800 is illustrated in FIG. 73. In FIG. 74, the operation of tool 1800 is illustrated. As current is applied from the current supply 1832 to the movement element 1820, the length of the movement element 1820 shortens. In some embodiments, the length of the movement element 1820 can shorten by approximately 5% of the length.

As current is repeated applied to and disconnected from the movement mechanism 1820, the length of the movement mechanism 1820 alternately adjusts along the directions of arrows “BK” and “BL.” As the coupler 1830 is moved in a similar manner, motion along the directions of arrows “BM” and “BN” is also imparted to distal end 1814 and tip 1816. This repeated motion of the cutting tip 1816 allows the cutting tip 1816 to moving in a scratching-like manner.

Referring to FIGS. 75-77, additional embodiments of tools that can be used according to the invention are illustrated. A functional block diagram is illustrated in FIG. 75. As shown, tool 1900 includes a support portion 1902 which can be configured to resemble a tube. Coupled to the support portion 1902 is a cutting element 1904 that includes one or more openings. A water supply 1906 is coupled to and supplied to the support portion 1902 under pressure. The water from supply 1906 passes through the support portion 1902 to the cutting element 1904 and exits the openings in the cutting element 1904 as a high velocity spray of air and water 1908. This spray 1908 functions as an abrasive means by which an endplate or facet joint articulating surface can be cut, eroded or abraded as desired. A physician can control the orientation of the cutting element 1904 to direct the spray 1908 in a desired manner. In an alternative embodiment, the water supply 1906 may be provided to the cutting element using a conduit that is not passed through the support portion 1902.

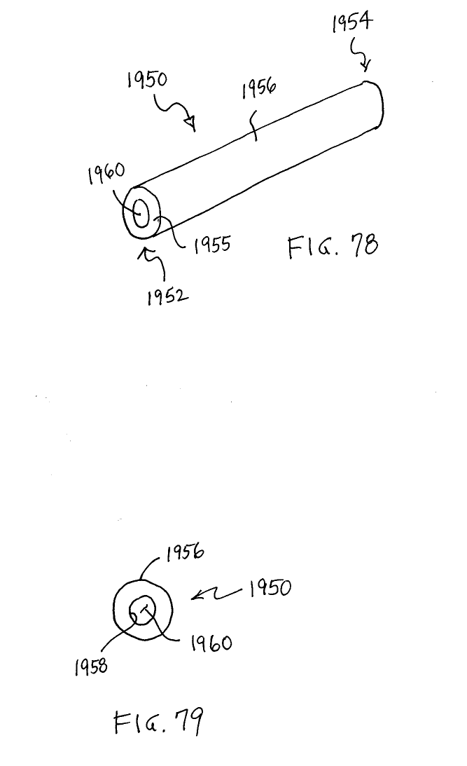

Referring to FIG. 76, an alternative embodiment of a tool is illustrated. In this embodiment, tool 1920 includes a support 1922 with a cutting element 1924 coupled to one end. The cutting element 1924 can have several openings through which water from a water supply 1926 can pass. The cutting element 1924 can be fixedly or movably mounted to the support 1922. If cutting element 1924 is movably mounted, it can be moved along the directions of arrow “BO” as shown in FIG. 76 to direct the spray 1928 toward the desired target area or region.

Referring to FIG. 77, another embodiment of a tool is illustrated. Tool 1940 includes a support portion 1942 with a cutting portion or element 1944 at one end. The cutting portion 1944 includes an additional component 1945, which in this embodiment is arcuate and in fluid communication with the support portion 1942 and the cutting portion 1944. Water from a water supply 1946 is provided to the support portion 1942 and cutting portion 1944. The water is pressurized and as a result, a high velocity spray of air and water 1948 exits openings formed in the arcuate component 1945.

Each of the cutting elements 1904, 1924, and 1944 illustrated in FIGS. 75-77 can be moved or adjusted by a physician to cause the corresponding spray 1908, 1928, and 1948 to engage the desired target area or region in a disc space.

In various embodiments, the materials and configurations of the components can vary depending on the properties and functionality desired for the particular component.

An alternative embodiment of a delivery device is illustrated in FIGS. 78-79. In this embodiment, the delivery device 1950 has a proximal end 1952 and a distal end 1954. The device 1950 has a substantially cylindrical, elongate body 1955 that extends along a longitudinal axis 1962 from the proximal end 1952 to the distal end 1954. The body 1955 has an outer surface 1956 and has an inner surface 1958 that defines a channel 1960 that extends along the length of the delivery device 1950. The outer surface 1956 defines an outer diameter of the body 1955. The inner surface 1958 defines an inner diameter that is the diameter of the channel 1960. Any preparation device or tool that is to be delivered to a location, such as a disc space, through the delivery device 1950 is limited in diameter by the diameter of the channel 1960.

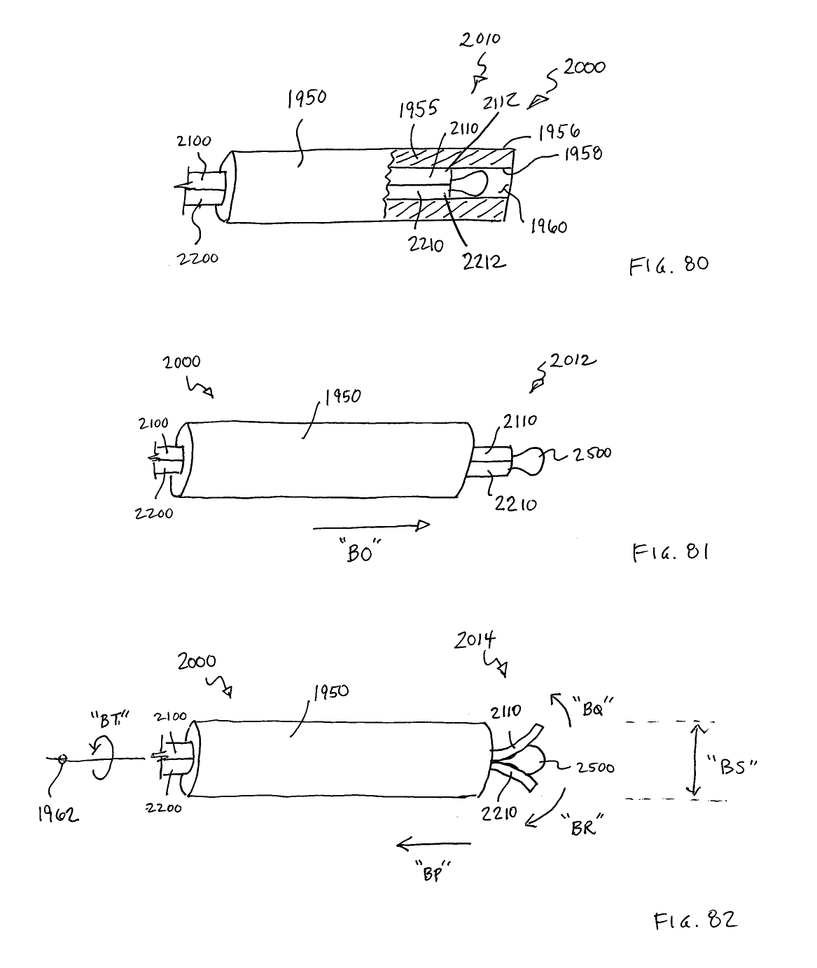

An alternative embodiment of a site preparation tool or device is illustrated in FIGS. 80-82. Referring to FIG. 80, a partial cross-sectional side view of delivery device 1950 is illustrated for ease of reference. The site preparation tool 2000 includes a first preparation device 2100 and a second preparation device 2200. The preparation devices are configured to engage, such as by cutting, one or more endplates. The cutting of an endplate can cause or induce blood flow into a disc space. The preparation devices can be referred to as cutting elements, cutting components, or collectively as a cutting mechanism.

Each of the preparation devices 2100 and 2200 is connected near its proximal end to a control device or mechanism that can be manipulated or controlled by a user. An exemplary control mechanism can be a drive mechanism with a power supply and a coupler or connection between the drive mechanism and the preparation device. When the drive mechanism is operated, motion, such as rotation, can be imparted to the preparation devices. Some exemplary control devices or mechanism include control portion 170, control portion 186, and drive mechanism 196 as discussed above.

The first preparation device 2100 includes a cutting element or portion 2110 proximate to its distal end 2112. Similarly, the second preparation device 2200 includes a cutting element or portion 2210 proximate to its distal end 2212. In FIG. 80, the preparation devices 2100 and 2200 are illustrated in delivery configurations 2010 in which the preparation devices 2100 and 2200 are proximate to each other. The preparation devices 2100 and 2200 in their delivery positions or configurations are aligned with the longitudinal axis of the tool and delivery device 1950 as shown. When the preparation devices 2100 and 2200 are in their delivery configurations 2010, the preparation devices 2100 and 2200 can be passed through the channel 1960 of the delivery device 1950 to a disc space.

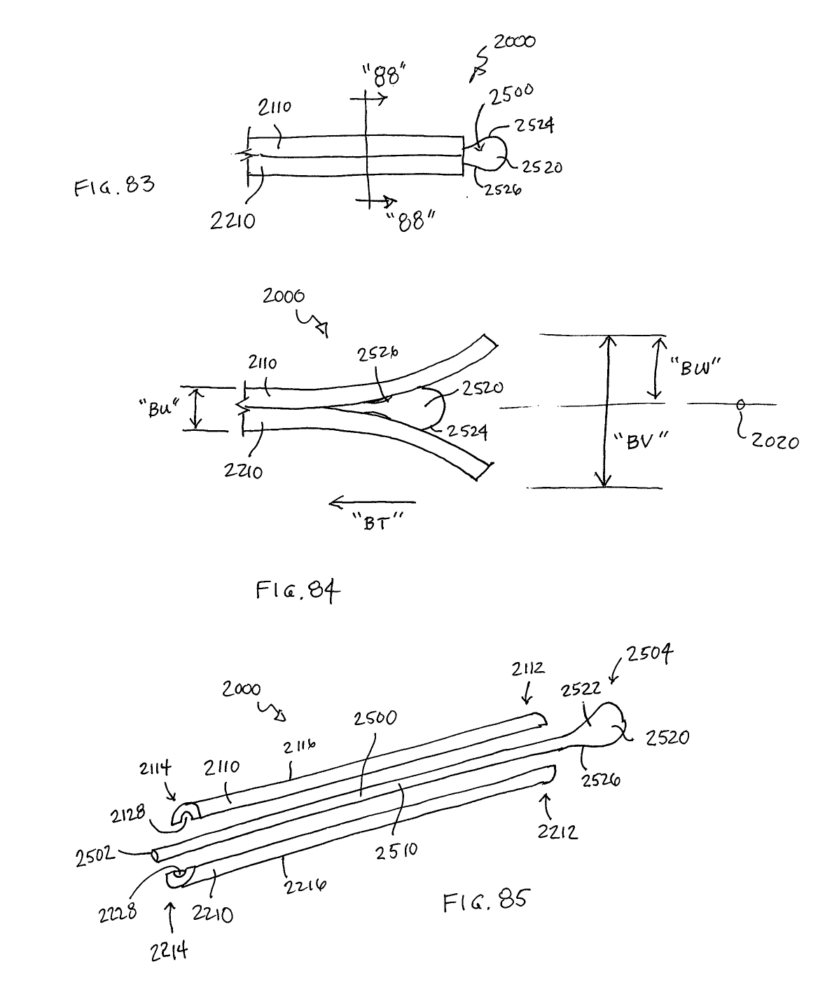

Referring to FIG. 81, preparation tool 2000 can be moved by a user along the direction of arrow “BO.” Movement along that direction results in the distal ends 2112 and 2212 of cutting elements 2110 and 2210 extending beyond the distal end 1954 of the delivery device 1950 in their deployed configurations 2012.

The preparation tool 2000 also includes an actuating component or element 2500. The actuating component 2500 can be manipulated to change the configuration of the cutting elements 2110 and 2210. The actuating component can be referred to alternatively as a deflecting element or device or an expanding element or mechanism. As described in detail below, the actuating component causes the cutting mechanism to expand. The terms “expanding” or “spreading apart” are used to reference the manner in which the cutting elements are moved. The terms “deflecting” and “angled” are used interchangeably to reference the surface on the actuator that is used to engage the cutting elements so that they expand or spread apart.

In both the initial and fully deployed configurations 2012 and 2014, a portion of the actuating component 2500 extends beyond the distal ends 2112 and 2212 of the cutting elements 2110 and 2210. As shown in FIGS. 80 and 81, the preparation devices 2100 and 2200 and the actuating component 2500 are moved together or substantially simultaneously through the delivery device 1950 to the desired location.

Referring to FIG. 82, the interaction between the actuating component 2500 and the cutting elements 2110 and 2210 is described. The actuating component 2500 is movable relative to the cutting elements 2110 and 2210. In this embodiment, the actuating component 2500 is movable along the direction of arrow “BP” in FIG. 82, which is substantially aligned with the longitudinal axis 1962 of the delivery device 1950. The positions of the cutting elements 2110 and 2210 shown in FIG. 82 are representative of the location at which the cutting of a vertebral endplate can occur.

As the actuating component 2500 moves along the arrow “BP,” the actuating component 2500 engages the cutting elements 2110 and 2210 substantially simultaneously and spreads them apart. As a result, the actuating component 2500 forces the cutting elements away from each other along the directions of arrows “BQ” and “BR,” respectively. The first portion of the cutting elements that engage the actuating component are their distal ends, which are free ends in that they are not connected to any structure.

The extent to which the ends of the cutting elements 2110 and 2210 extend outwardly (illustrated as distance “BS”), depends on several factors. One factor is the distance that the cutting elements 2110 and 2210 extend beyond the distal end 1954 of the delivery device 1950. The farther that the cutting elements 2110 and 2210 extend enables the degree of expansion or expansion distance “BS” of the cutting elements 2110 and 2210 to increase. Another factor is the flexibility of the material of the cutting elements 2110 and 2210. Increased flexibility of the material facilitates the bending of the cutting elements 2110 and 2210.

Another factor is the distance that the actuating component 2500 is moved along arrow “BP” relative to the cutting elements 2110 and 2210. The greater the distance that the actuating component 2500 is moved relative to the cutting elements 2110 and 2210, the wider the cutting elements 2110 and 2210 can be spread apart. Another factor is the shape of the actuating component. As the actuating component is pulled between the cutting elements, the shape will affect the expansion as described below.

When the cutting elements 2110 and 2210 are spread apart in their deployed configurations 2014 as shown in FIG. 82, a user can manipulate the preparation devices or tools 2100 and 2200 to rotate them about longitudinal axis 1962 along arrow “BT.” The tools 2100 and 2200 can be rotated in one direction, rotated in the opposite direction, and/or alternately rotated in opposite directions. The rotation of the preparation tools 2100 and 2200 causes the ends or cutting surfaces of the cutting elements 2110 and 2210 to engage one or more endplates or other structures as desired.

In one embodiment, the preparation tools 2100 and 2200 and the actuating component 2500 are rotated simultaneously with each other about axis 1962. In another embodiment, the preparation tools 2100 and 2200 can be rotated about the actuating component 2500. A user can rotate the preparation devices 2100 and 2200 by hand by gripping a handle or control mechanism and manually rotating the device. Alternatively, a user can operate a drive mechanism to achieve the desired movement.

Referring to FIGS. 83-85, the actuating component 2500 is illustrated relative to the cutting elements 2110 and 2210. As shown in FIG. 85, actuating component 2500 has a distal end 2502 and a proximal end 2504. Actuating component 2500 includes a support portion 2510 and an actuator or actuating portion 2520. In this embodiment, the support portion 2510 and the actuator 2520 are integrally formed. In other embodiments, the support portion 2510 and the actuator 2520 can be formed separately and coupled together using any conventional technique or method. The actuator 2520 includes a body portion 2522 that has an outer surface 2524. The outer surface 2524 of the body portion 2522 has a width that is greater than the outer diameter of the support portion 2510. As described below, the body portion 2522 can have various shapes and configurations in different embodiments.

The body portion 2522 includes an angled or deflector surface 2526 that is engaged by the distal ends and then the inner surfaces of the cutting elements 2110 and 2210. The particular configuration and orientation of the deflector surface 2526 can vary.

Referring to FIG. 84, in this embodiment, when the cutting elements 2110 and 2210 are disposed proximate to each other, they have an outer diameter “BU.” In one embodiment, this outer diameter can be in the range of 0.5 mm to 3.4 mm when the cutting elements 2110 and 2210 are in their delivery configurations. In other embodiments, this outer diameter can be in the range of 1.0 mm to 2.0 mm. As the actuating component 2500 is moved along the direction of arrow “BT,” the deflector surface 2526 of the actuator 2520 engages the cutting elements 2110 and 2210. As the actuating component 2500 continues to move, the outer surface 2524 of the body portion 2522 continues to engage the inner surfaces of the cutting elements 2110 and 2210. As the wider body portion 2522 moves between the cutting elements 2110 and 2210, the cutting elements 2110 and 2210 are forced outwardly. The cutting elements in their deployed positions or configurations extend away from the longitudinal axis of the tool and delivery device.

In the positions shown in FIG. 84, the outward tips and ends of the cutting elements 2110 and 2210 extend the distance of “BV,” with each cutting element extending approximately a distance “BW” from the longitudinal axis 2020 of the actuating component 2500. As shown, the distance “BV” is greater than the distance “BU” as well as the outer diameter of the delivery device 1950. In other words, in its delivery configuration (see FIG. 83), the cutting mechanism has a profile that is smaller than the profile of the cutting mechanism in the deployed configuration (see FIG. 84). As a result, the cutting elements 2110 and 2210 have a wider range of motion as the device or tool 2000 is rotated. The increased range of motion enables the cutting elements 2110 and 2210 to engage and contact additional surfaces and structures in the disc space, including those farther away from the tool. In addition, a wider diameter of the tool in the deployed configuration provides more material with which to cut. In one embodiment, the diameter of the cutting mechanism in its deployed configuration can be in the range of 1.0 times the outer diameter of the delivery device 1950 to 3.0 times the outer diameter of the delivery device 1950.

Referring to FIG. 85, an exploded perspective view of an embodiment of a site preparation tool is shown. In particular, some of the features of the embodiments of the preparation devices 2100 and 2200 are illustrated.

Preparation device 2100 has a distal end 2112 and a proximal end 2114. In this embodiment, preparation device 2100 is an elongate member, such as a wire, that has an arcuate cross-sectional shape. The device 2100 has an outer surface 2116 and an inner surface 2118 that defines a groove 2128. Similarly, preparation device 2200 has a distal end 2212 and a proximal end 2214. Device 2200 has an outer surface 2216 and an inner surface 2218 that defines a groove 2228.

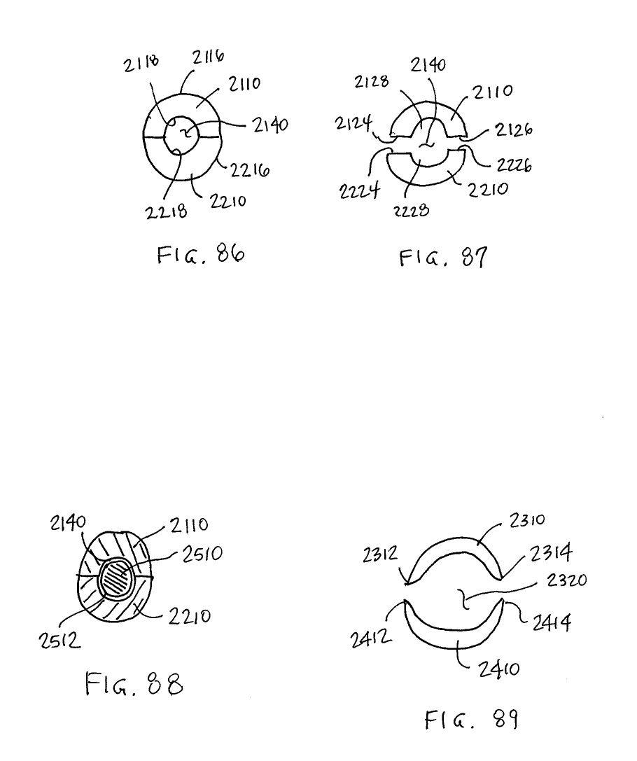

Referring to FIG. 86, an end view of the cutting elements 2110 and 2210 is illustrated. The actuating component 2500 is not illustrated relative to cutting elements 2110 and 2210 for ease of reference only. In FIG. 86, the cutting elements 2110 and 2210 are illustrated as being disposed proximate to each other, or in other words, in contact with each other. This arrangement corresponds to a delivery configuration of the cutting elements. The inner surface 2118 of element 2110 and the inner surface 2218 of element 2210 collectively form a channel 2140. The support portion 2510 of the actuating component 2500 is disposed in the channel 2140 (see FIG. 88 which is a cross-sectional view taken in FIG. 83). In this embodiment, the cutting elements 2110 and 2210 contact each other in their delivery configurations. In other embodiments, the cutting elements do not necessarily contact each other in the delivery configurations.

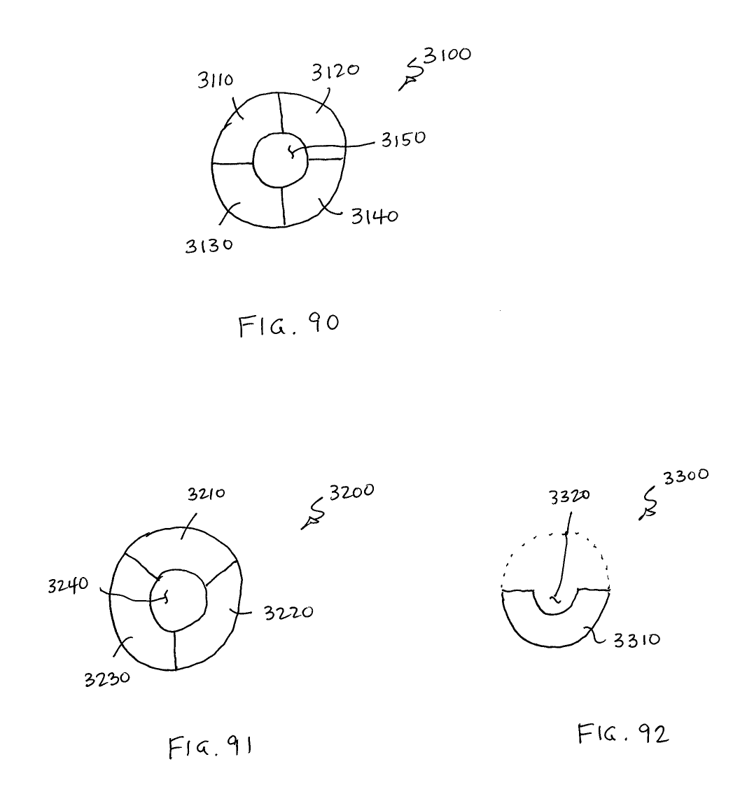

Referring to FIG. 87, an end view of the cutting elements 2110 and 2210 in a deployed configuration is illustrated. In this configuration, the cutting elements 2110 and 2210 are spaced apart from each other. As shown, end surfaces 2124 and 2126 of cutting element 2110 and end surfaces 2224 and 2226 of cutting element 2210 are spaced apart from and are not in contact with each other. The edges of the end surfaces 2124, 2126, 2224, and 2226 can be machined or modified to have a sharp edge that can be used as a cutting edge to engage an endplate during a procedure. In alternative embodiments, abrasive materials or particles can be adhered or coupled to the cutting elements if desired to increase the cutting ability.

In the illustrated embodiment, cutting elements 2110 and 2210 are substantially arcuate in cross-section and collectively have a configuration resembling a tube. The shape and configuration of the cutting elements can vary in different embodiments.

An alternative embodiment of cutting elements is illustrated in FIG. 89. In this embodiment, the cutting elements 2310 and 2410 have a different cross-sectional shape than cutting elements 2110 and 2210. Cutting element 2310 has cutting edges 2312 and 2314 and cutting element 2410 has cutting edges 2412 and 2414. The cutting elements 2310 and 2410 form a channel 2320 therebetween.

End views of other embodiments of cutting elements are illustrated in FIGS. 90-92. Referring to FIG. 90, the site preparation tool 3100 includes four cutting elements. In this embodiment, cutting elements 3110, 3120, 3130, and 3140 have substantially similar configurations and collectively define a channel 3150 through which a support portion of an actuating component can be disposed. The cutting elements 3110, 3120, 3130, and 3140 can be engaged by an actuating component and are spread apart radially and outwardly as previously described.

Referring to FIG. 91, in this embodiment, the site preparation tool 3200 includes three cutting elements. Cutting elements 3210, 3220, and 3230 have substantially similar configurations and collectively define a channel 3240 through which a support portion of an actuating component can be disposed. Cutting elements 3210, 3220, and 3230 can be spread apart by an actuating component or expander mechanism.

Referring to FIG. 92, in this embodiment, the site preparation tool 3300 includes one cutting element 3310 that defines a groove or channel 3320 through which a support portion of an actuating component is disposed. The cutting element 3310 can be redirected or deflected outwardly by an actuating component. In other embodiment, any quantity of cutting elements can be used in a site preparation tool.

Referring to FIGS. 93-97, different embodiments of actuating components or expansion mechanisms are illustrated. Each of the actuating components can have an actuator with a body portion. The shape, size or configuration of the body portion can vary.

As shown in FIG. 93, actuating component 2500 has a support portion 2510 and a body portion 2522 that defines a deflection surface 2526. In this embodiment, the body portion 2522 can have a generally oval configuration. The deflection surface 2526 extends around the perimeter of the body portion 2522. As a result, cutting elements may engage the deflection surface 2526 on any side of the body portion 2522. The angle “BX” defined by the deflection surface 2526 relative to the longitudinal axis 2020 can vary.

In some embodiments, the range of the angle “BX” is approximately 45 degrees to 80 degrees. The angle “BX” can be any angle in the range from greater than 0 degrees to less than 90 degrees. Such a range is based on the fact that to facilitate the movement of the cutting elements laterally, the body portion 2522 has to be wider than or have a greater dimension, such as width, than the support portion 2510 of the actuating component 2500 (in which case, the deflection surface 2526 is at an angle greater than 0 degrees relative to the longitudinal axis). In addition, in most cases, the angle should be less than 90 degrees so that the ends of the cutting elements are able to slide or move outwardly radially.

In other embodiments, the angle can be greater than 90 degrees provided that the cutting elements are pre-curved or bent. The curvature of the cutting elements facilitates the expansion of the cutting elements as they are contacted by an actuating component. In the various embodiments, the cutting elements can be formed of a shape member alloy, such as NITINOL, or stainless steel.

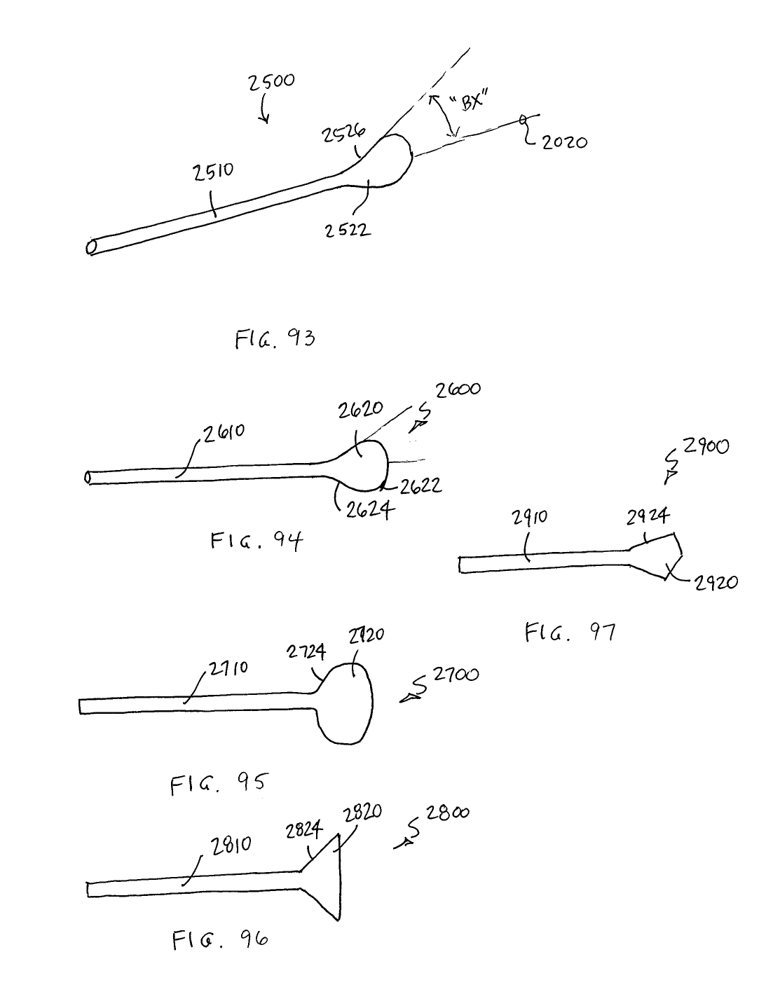

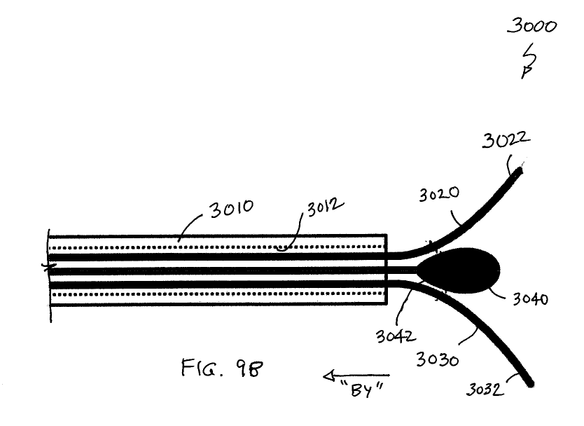

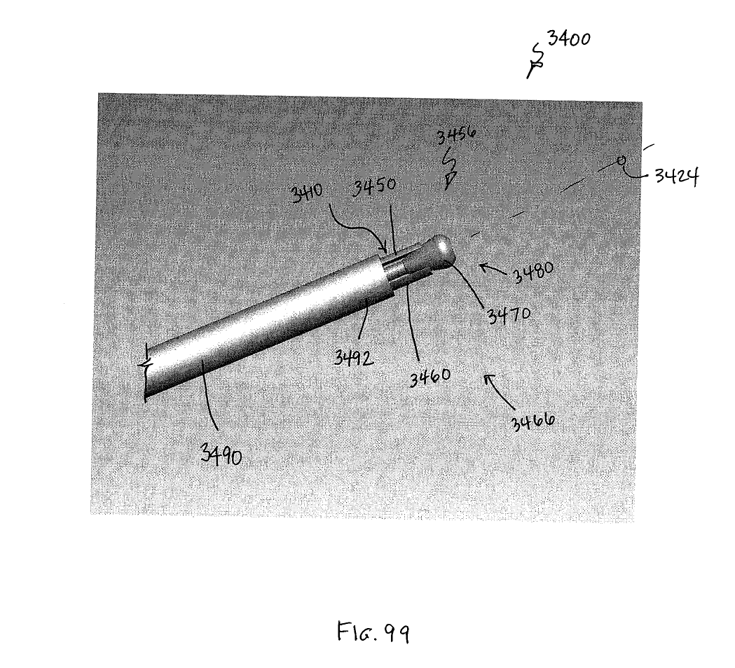

Referring to FIG. 94, the actuating component 2600 includes a support portion 2610 and a body portion 2620 that is generally circular. The body portion 2620 has an outer surface 2622 that has a deflection surface 2624 that is oriented at a smaller or gradual angle relative to the longitudinal axis.

Referring to FIG. 95, in this embodiment, the actuating component 2700 includes a support portion 2710 and a body portion 2720 that is generally spherical. The width of the body portion 2720 is greater than the width of the body portion 2620. As a result, the angle of the deflection surface 2724 relative to the longitudinal axis is larger than that of the deflection surface 2624. The larger angle causes the cutting elements to spread outwardly and expand more quickly.

Referring to FIG. 96, in this embodiment, the actuating component 2800 includes a support portion 2810 and a body portion 2820 with a deflection surface 2824. The deflection surface 2824 is oriented at approximately 45 degrees with respect to the longitudinal axis of the actuating component 2800.

Referring to FIG. 97, in this embodiment, the actuating component 2900 includes a support portion 2910 and a body portion 2920 with a deflection surface 2924. The angle of orientation of deflection surface 2924 is less than the angle of orientation of deflection surface 2824 and as a result, cutting elements engaging deflection surface 2924 are likely to spread apart more gradually than the cutting elements that engage deflection surface 2824.

A schematic view of an embodiment of a site preparation tool is illustrated in FIG. 98. In this embodiment, the site preparation tool 3000 includes cutting elements 3020 and 3030 that are configured to move through a channel 3012 in delivery device 3010. In this embodiment, the actuator 3040 has a tear drop shape. As actuator 3040 is moved along the direction of arrow “BY,” the deflecting surface 3042 engages cutting elements 3020 and 3030 and cutting ends or tips 3022 and 3032 are moved outwardly. In FIG. 98, the actuator 3040 has already engaged the free ends of the cutting elements 3020 and 3030 and spread them apart.

During a procedure, the delivery device 1950 is inserted so that the distal end 1954 is located in the particular disc space. The actuating component 2500 is located within the cutting mechanism or between cutting elements 2110 and 2210. The cutting mechanism or cutting elements and the actuating component 2500 are then moved through the channel 1960 of the delivery device to a desired location.

The actuating component 2500 is pulled back and engages the distal ends of the cutting elements. As the distal ends engage the deflection surface of the actuator, the cutting elements spread outwardly. The preparation tool is manipulated so that the cutting elements engage one or both vertebral endplates that define part of the disc space. When the procedure is finished, the actuating component is pushed distally. Once the actuator body portion is beyond the distal ends of the cutting elements, the cutting elements return to their delivery configurations. The cutting elements and actuating component can be pulled into the delivery device and withdrawn from the patient.

In one embodiment, the body portion of the actuator has a generally symmetrical or uniform shape or configuration around its perimeter. In other embodiments, the shape or configuration of the body portion doe s not have to be symmetrical. A non-symmetrical shape or configuration will result in the body portion engaging the cutting elements at different times and to different extents.

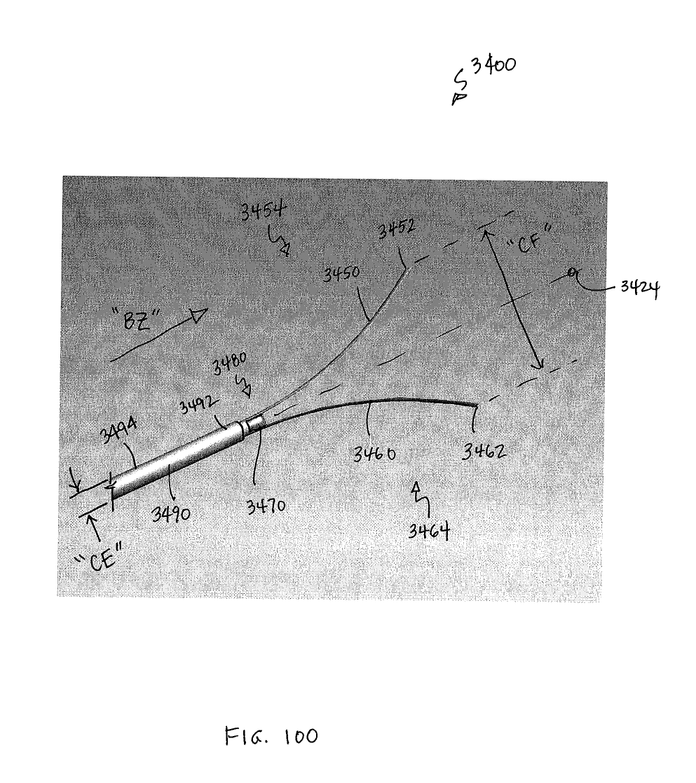

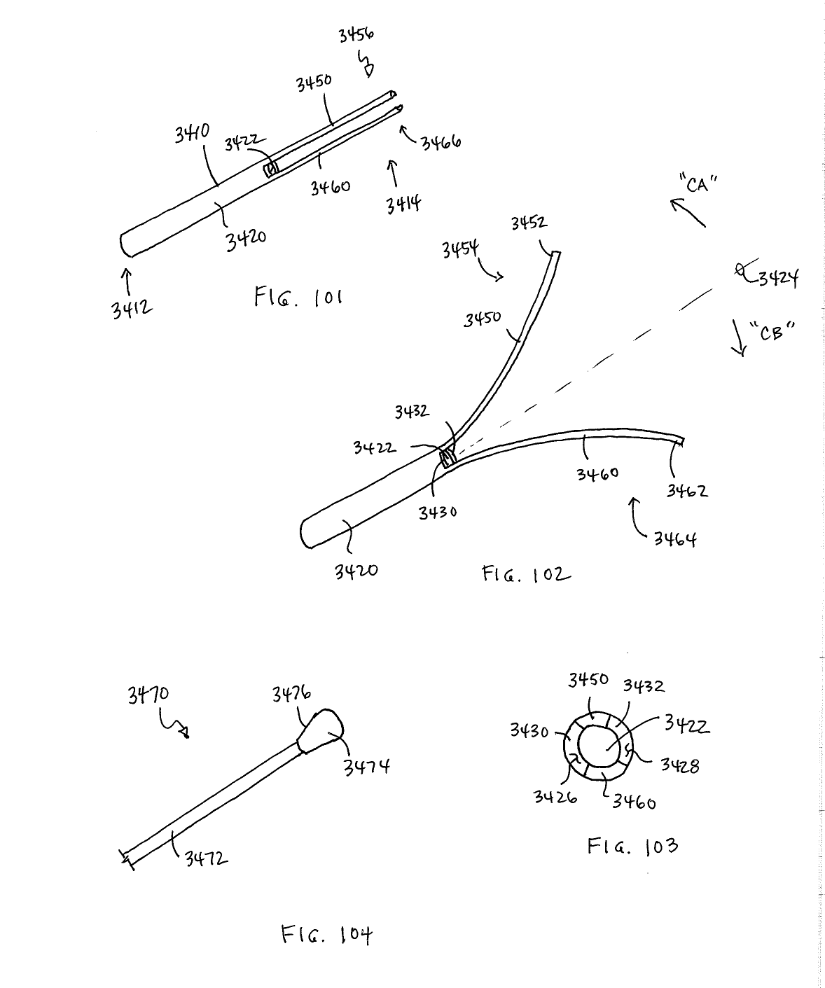

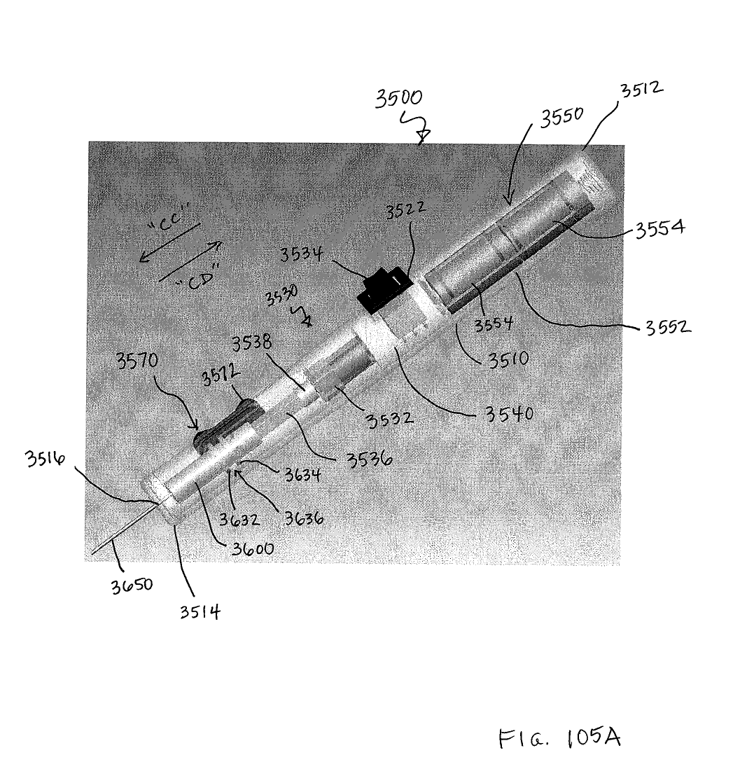

An alternative embodiment of a site preparation tool or device is illustrated in FIGS. 99-104. Referring to FIGS. 99 and 100, site preparation tool 3400 is illustrated in a delivery configuration and in a deployed configuration, respectively. The site preparation tool 3400 includes a cutting tool or portion 3410 and an actuating component 3470. The cutting tool 3410 and actuating component 3470 are configured to be inserted through a channel (not shown) in the delivery device or cannula 3490. The cutting tool 3410 and actuating component 3470 are illustrated as extending from the distal end 3492 of the cannula 3490 in FIGS. 99 and 100. As shown in FIG. 100, the delivery device 3490 has an outer surface 3494 that defines an outer diameter “CE” of the delivery device 3490.

Referring to FIG. 99, the cutting tool 3410 includes a first cutting element or preparation device 3450 and a second cutting element or preparation device 3460. The cutting elements 3450 and 3460 are configured to engage an actuator 3470 which can be fixed or secured in an extended position 3480 relative to the distal end 3492 of the delivery device 3490.

Each of the cutting elements 3450 and 3460 is connected near its proximal end to a control device or mechanism that can be manipulated or controlled by a user. An exemplary control mechanism can be a drive mechanism with a power supply and a coupler or connection between the drive mechanism and the cutting elements. When the drive mechanism is operated, motion, such as rotation, can be imparted to the cutting elements. In addition, a user controllable actuator may be provided to move the cutting elements back and forth along a longitudinal direction. Some exemplary control devices or mechanism include control portion 170, control portion 186, and drive mechanism 196 as discussed above or control mechanism 3500 as described below.

Cutting element 3450 includes a cutting tip or portion 3452 proximate to its distal end. Similarly, the cutting element 3460 includes a cutting tip or portion 3462 proximate to its distal end. In FIG. 99, the cutting elements 3450 and 3460 are illustrated in delivery configurations 3456 and 3466, respectively, in which the cutting elements 3450 and 3460 are proximate to each other. The cutting elements 3450 and 3460 in their delivery positions or configurations are aligned with the longitudinal axis 3424 of the tool 3400 and delivery device 3490 as shown. When the cutting elements 3450 and 3460 are in their delivery configurations 3454 and 3464, the cutting elements 3450 and 3460 can be passed through the channel of the delivery device 3490 to a disc space or facet joint.

Referring to FIG. 100, the preparation tool 3400 can be moved by a user along the direction of arrow “BZ.” Movement along that direction results in the distal ends of cutting elements 3450 and 3460 moving to their deployed configurations 3454 and 3464, respectively. The extent to which the cutting elements 3450 and 3460 extend in their deployed configurations 3454 and 3464 is determined by the distance that the cutting tool 3410 is moved along the direction of arrow “BZ.”

The preparation tool 3400 also includes an actuating component or element 3470. The actuating component 3470 is used to change the configuration of the cutting elements 3450 and 3460. The actuating component 3470 can be referred to alternatively as a deflecting element or device or an expanding element or mechanism. As described in detail below, the actuating component 3470 causes the cutting elements 3450 and 3460 to expand. As set forth above, the terms “expanding” or “spreading apart” are used to reference the manner in which the cutting elements are moved and the terms “deflecting” and “angled” are used interchangeably to reference the surface on the actuator that is used to engage the cutting elements so that they expand or spread apart.

In a delivery or an initial deployed configuration (see FIG. 99), a portion of the actuating component 3470 extends beyond the distal ends of the cutting elements 3450 and 3460. The cutting tool 3410 and the actuating component 3470 are moved together or substantially simultaneously through the delivery device 3490 to the desired location.

Referring to FIG. 100, the interaction between the actuating component 3470 and the cutting elements 3450 and 3460 is shown. The actuating component 3470 is fixed in place (such as in position 3480) relative to the distal end 3492 of the delivery device 3490. The actuating component 3470 can be maintained in position 3480 in several different ways, including mechanically, such as by welding, automatically, and/or manually by a user.

As the cutting tool 3410 moves along the direction of arrow “BZ,” the cutting elements 3450 and 3460 are moved outwardly away from the longitudinal axes 3424 of the cutting tool 3410 and the delivery device 3490. As the cutting elements 3450 and 3460 move outwardly, the cutting ends or tips 3452 and 3462 move away from each other as illustrated in FIG. 100. As shown, the cutting elements 3450 and 3460 in their deployed positions define an outer diameter “CF.” In the positions illustrated in FIG. 100, the outer diameter “CF” defined by the deployed cutting elements 3450 and 3460 is larger than the outer diameter “CE” of the delivery device 3490. In one embodiment, the diameter or outer diameter “CF” of the cutting mechanism or cutting elements 3450 and 3460 in its deployed configuration or positions can be in the range of 1.0 times the outer diameter “CE” of the delivery device 3490 to 3.0 times the outer diameter “CE” of the delivery device 3490. In addition, the outer diameter “CF” of the cutting elements can vary depending on the patient in which the cutting tool is used. In some patients, the extent to which the cutting elements are able to expand may be limited by the dimensions of the particular disc spaces or facet joints. For a disc space in which the cutting tool is to be inserted that is relatively small in size, the cutting elements may be limited by the proximity of the endplates defining the disc space. For a disc space that is relatively larger, the cutting elements may expand beyond 3.0 times the outer diameter “CE” of the delivery device.

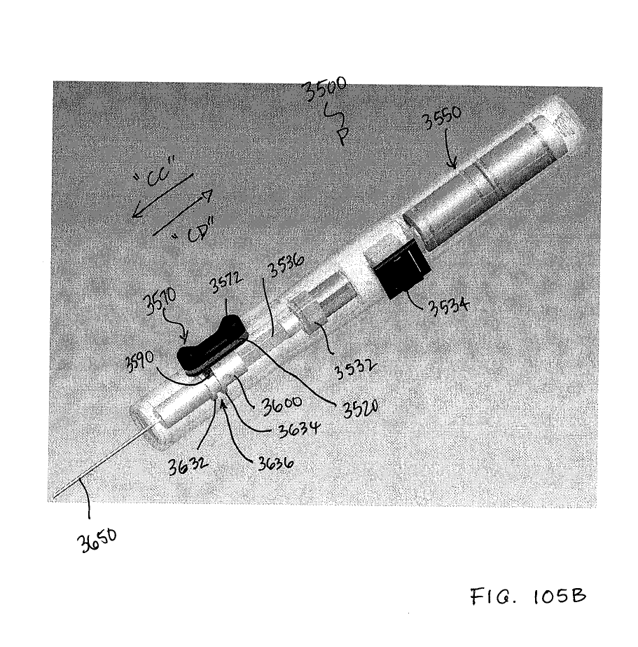

Referring to FIGS. 101-103, an embodiment of the cutting tool 3410 is illustrated. As shown in FIG. 101, the cutting tool 3410 includes a proximal end 3412 and a distal end 3414 that is located away from the user. The cutting tool 3410 includes a body 3420 that has a channel 3422 extending therethrough and a longitudinal axis 3424. Notches or slots 3426 and 3428 (see FIG. 103) are formed in the cutting tool 3410, which in one embodiment, can initially be a tube. End surfaces 3430 and 3432 are located at the ends of the notches or slots 3426 and 3428, which are formed by machining the tube to remove the desired amount of material.

Referring to FIG. 103, the end surfaces 3430 and 3432 are shown. The removal of material results in the formation of cutting elements 3450 and 3460. As shown in FIG. 103, the size of the cutting elements 3450 and 3460 is determined by the amount of material that is removed. The larger the slots 3426 and 3428 are results in narrower cutting elements 3450 and 3460, which in turn results in increased flexibility of the cutting elements 3450 and 3460, making them easier to move outwardly. The more flexible that the cutting elements 3450 and 3460 are results in a lower amount of force required to spread the cutting elements 3450 and 3460 and ends 3452 and 3462 from their delivery configurations 3456 and 3466 (see FIG. 101) to their deployed configurations 3454 and 3464 (see FIG. 100).

As the cutting elements 3450 and 3460 substantially simultaneously engage the actuating component or actuator 3470, the cutting elements 3450 and 3460 are spread apart and forced away from each other along the directions of arrows “CA” and “CB,” respectively (see FIG. 102). The first portions of the cutting elements 3450 and 3460 that engage the actuating component 3470 are the distal ends 3452 and 3462, which are free ends in that they are not connected to any structure.

Referring to FIG. 104, an embodiment of an actuating component or actuator 3470 is illustrated. The actuator 3470 includes a support portion 3472 with a body portion 3474 having a deflecting surface 3476 that is configured to be engaging by the cutting elements 3450 and 3460. As described above relative to other embodiments, the size, shape and/or configuration of the body portion 3474 and the deflecting surface 3476 can vary in different embodiments of the actuator.

Referring to FIGS. 105A-109, an embodiment of a control mechanism 3500 is illustrated. In this embodiment, the control mechanism 3500 can be manipulated by a user to perform the desired procedure on a patient. As shown in FIGS. 105A and 105B, a delivery device 3650 extends from an end of the control mechanism 3500. A cutting tool (not shown), similar to cutting tool 3410 previously described, can be deployed from or otherwise extend from the delivery device 3650.

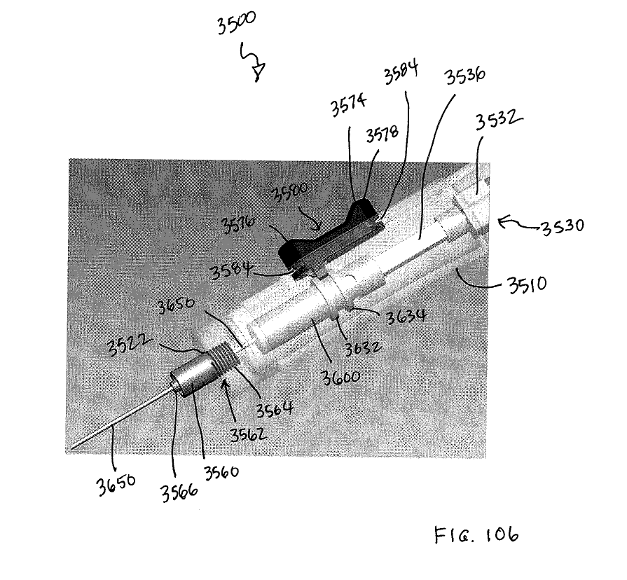

Control mechanism 3500 includes a housing 3510 with a proximal end 3512 and a distal end 3514. For ease in description and explanation, the housing 3510 is illustrated as being transparent so that the internal components of the control mechanism 3500 can be viewed. In one embodiment, the distal end 3514 can include a small opening 3516 (see FIGS. 105A and 107) through which a delivery device 3650 can extend. In another embodiment, the distal end 3514 can include a larger opening 3522 into which a luer lock 3560 can be inserted (see FIG. 106). In one embodiment, the luer lock 3560 can include a mounting portion 3562 with threads 3564 and an extending portion 3566. As shown in FIG. 107, the housing 3510 can also include an outer surface 3518 in which a slot or opening 3520 is formed, the function of which is described below.

In this embodiment, the housing 3510 includes a power supply 3550 that is disposed in a compartment 3552 formed in the housing 3510 (see FIGS. 105A and 105B). The power supply 3550 can be one or more sources of power, such as cells, batteries (see batteries 3554 in FIG. 105A), etc. In one embodiment, the power supply 3550 can be two 3-volt batteries. Alternatively, the control mechanism 3500 can be powered by an external power supply.

The control mechanism 3500 includes a drive mechanism 3530 with a motor or drive 3532 that is coupled to an output shaft 3536. An electronic housing 3540 is provided in which various electronic components, including wiring, can be disposed. A button or switch 3534 is disposed in an opening 3522 formed in the housing 3510 and is operably connected to the motor 3532 so that a user can activate the motor 3532 by pressing on the button 3534. The output shaft 3536 is rotatably supported in a sleeve 3538. The output shaft 3536 is connected to the cutting tool so that the user can rotate the cutting tool, including any cutting elements, by activating the motor 3532.

The control mechanism 3500 includes an actuator 3570, which can be referred to as an extender or slider. The actuator 3570 can be manipulated by a user to move the cutting tool and cutting elements from a delivery or retracted configuration to a deployed or extended configuration. In addition, the actuator 3570 can be manipulated to move the cutting tool and cutting elements from a deployed configuration to a delivery configuration. In particular, the actuator 3570 can be moved along the direction of arrow “CC” in FIGS. 105A and 105B to extend or deploy a cutting tool and cutting elements. The actuator 3570 can also be moved along the direction of arrow “CD” in FIGS. 105A and 105B to retract or withdraw a cutting tool and cutting elements.

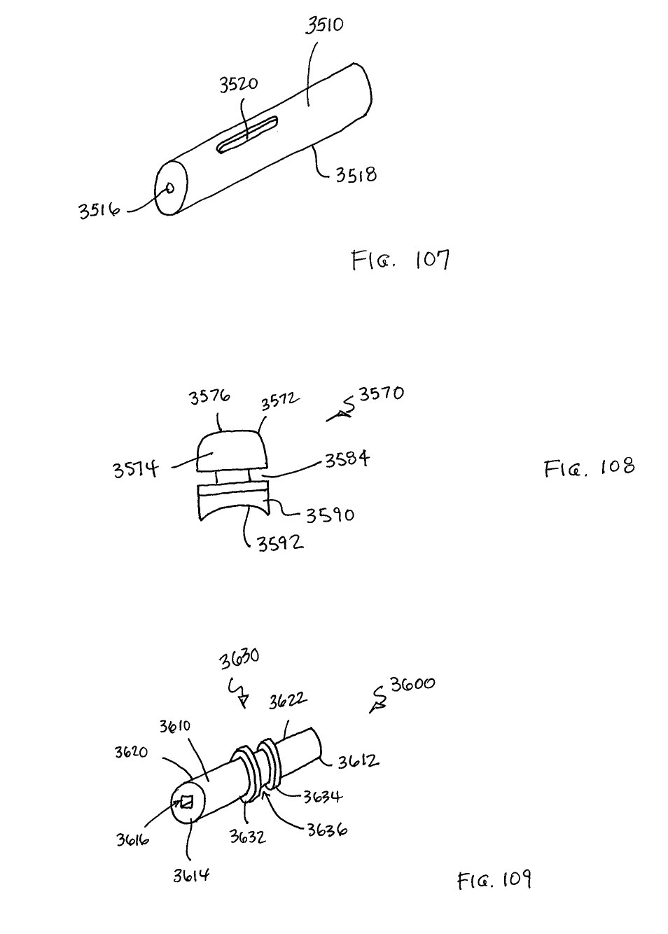

Referring to FIGS. 105A, 105B, 106 and 108, the actuator or actuating mechanism 3570 includes a slider or sliding mechanism 3572 with an engaging portion or body 3574 with ridges 3576 and 3578 and a groove 3580 formed in between. The groove 3580 is configured to receive a portion of the user's finger or thumb to move the slider 3572. The body 3574 includes a slot or groove 3584 that extends around the perimeter of the body 3574. The lower portion of the body 3574 includes a control portion or extension 3590 that in one embodiment includes a curved surface 3592 (see FIG. 108). The slider 3572 can be formed of any material, including molded plastic.

The actuator 3570 is configured to engage a controller or sleeve 3600 that is slidably disposed on output shaft 3536. As shown in FIGS. 105A, 105B, 106, and 109, the sleeve 3600 includes a body 3610 with ends 3612 and 3614 and a channel 3616 extending therethrough from end 3612 to end 3614. Channel 3616 has a shape or configuration that cooperates with the shape or configuration of the output shaft 3536. For example, the output shaft 3536 can have a square cross-section and the channel 3616 in sleeve 3600 can have a square cross-section. The cooperating cross-sections enable rotation of the output shaft 3536 to rotate the sleeve 3600 at the same time.

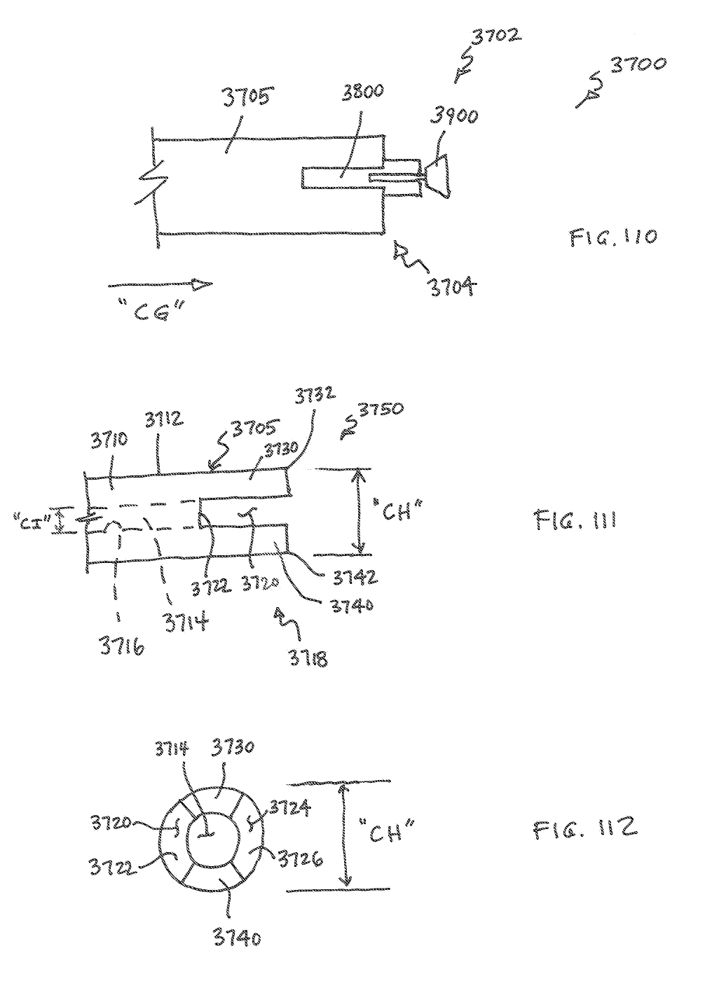

The body 3610 of the controller or sleeve 3600 has an outer surface 3620 that defines a perimeter 3622. The body 3610 also includes an engaging portion 3630 (see FIG. 109). The engaging portion 3630 includes spaced apart ribs 3632 and 3634 that define a space or groove 3636 therebetween. The ribs 3632 and 3634 extend around the perimeter 3622 of the body 3610. The engaging portion 3590 on the actuator 3570 engages the groove 3636 and the curved surface 3592 of the actuator 3570 contacts the outer surface 3620 of the body 3610. Thus, as the sleeve 3600 rotates (when driven by the motor 3532), the engaging portion 3590 on the actuator 3570 slides along the outer surface 3620 in the groove 3636, and the rotation of the sleeve 3600 is not impeded by the engaging portion 3590.

As a user moves the actuator 3572 along the direction of either arrow “CC” or arrow “CD,” the user moves the actuator 3572 along the slot 3520 in the housing 3510 and the engaging portion 3590 moves the sleeve 3600 along the output shaft 3536 in the same direction. Thus, while the motor 3532 rotates the sleeve 3600 and a cutting tool, such as cutting tool 3410, a user can extend or retract the cutting tool simultaneously by moving the actuator or slider 3572. This dual movement arrangement can be used to increase the working area of the cutting tool when it is deployed in the desired work space by allowing a user to rotate a cutting tool while extending and retracting the cutting tool at the same time.

In various alternative embodiments, the shapes or configurations of the actuators, sleeve, drive shaft, luer lock and other components illustrated in FIGS. 99-109 can vary.

Referring to FIG. 110, a side view of a portion another embodiment of a site preparation tool in accordance with an aspect of the present invention is illustrated. In this embodiment, the site preparation tool 3700 includes a cutting tool 3705 and an expander or expanding mechanism that includes two expander portions 3800 and 3900. Only a portion of the components of site preparation tool 3700 are illustrated in FIGS. 110-117. Each of the expander portions 3800 and 3900 can be referred to alternatively as an expander or expanding mechanism. In addition, the expanding mechanism can be referred to as a deflecting element.

In FIG. 110, the site preparation tool 3700 is illustrated in a delivery configuration 3702 in which the site preparation tool 3700 can be moved through a delivery device along the direction of arrow “CG” so that the distal end 3704 of the site preparation tool 3700 is disposed at the desired location in a patient.

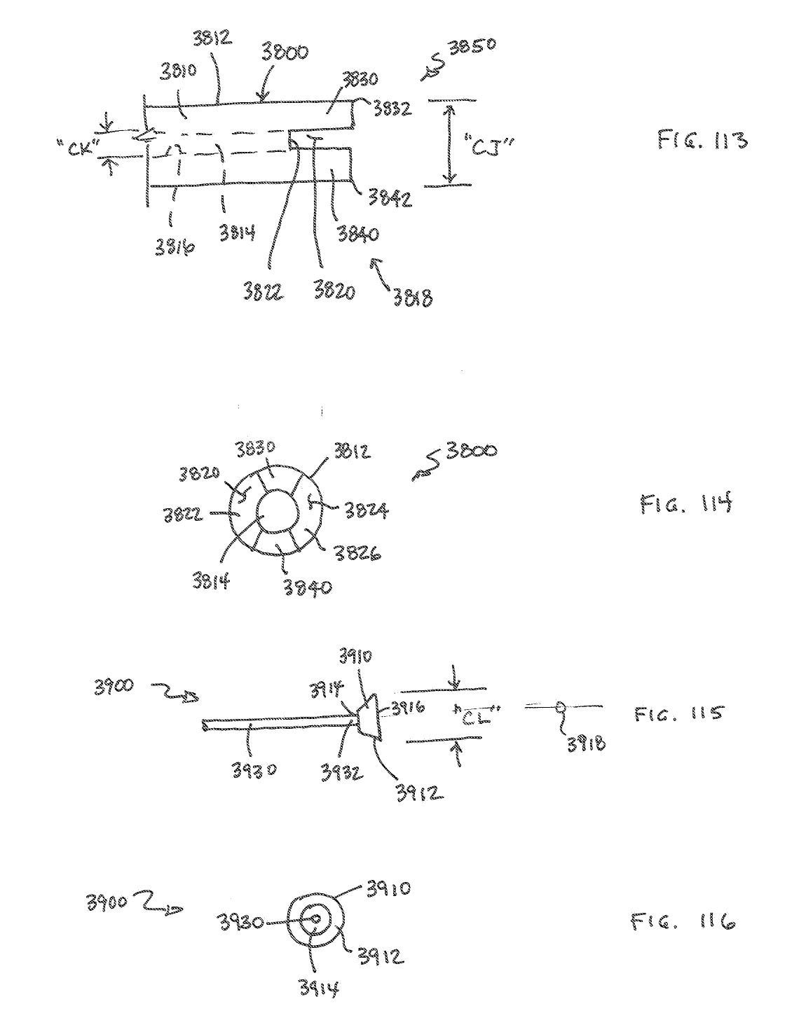

Referring to FIGS. 111 and 112, a side view and an end view of an embodiment of the cutting tool of site preparation tool 3700 are illustrated, respectively. In this embodiment, the cutting tool 3705 includes a body 3710 with an outer surface 3712 that has a diameter “CH” as shown in FIG. 111 in its delivery configuration 3750. The body 3710 includes a channel 3714 that is defined by inner surface 3716. The channel 3714 extends through the body 3710 to the distal end 3718 of the body 3710. In this embodiment, the channel 3714 defines an inner diameter “CI” of the body 3710.

The cutting tool 3705 includes cutting elements 3730 and 3740. As shown in FIGS. 111 and 112, notches 3720 and 3724 are formed in the body 3710 and extend to surfaces 3722 and 3726, respectively. The notches 3720 and 3724 are formed by the removal of material and they allow the cutting elements 3730 and 3740 to move relative to the body 3710 of the cutting tool 3705. The cutting elements 3730 and 3740 include tips or edges 3732 and 3742, respectively, that are used in a cutting or traumatizing process as described herein.

Referring to FIGS. 113-116, an embodiment of the expander or expanding mechanism of the site preparation tool 3700 is illustrated. This expander or expanding mechanism can be referred to as a two-stage or multi-stage expanding mechanism as it includes multiple portions or parts that cooperate to expand the cutting elements 3730 and 3740 of the cutting tool 3700.

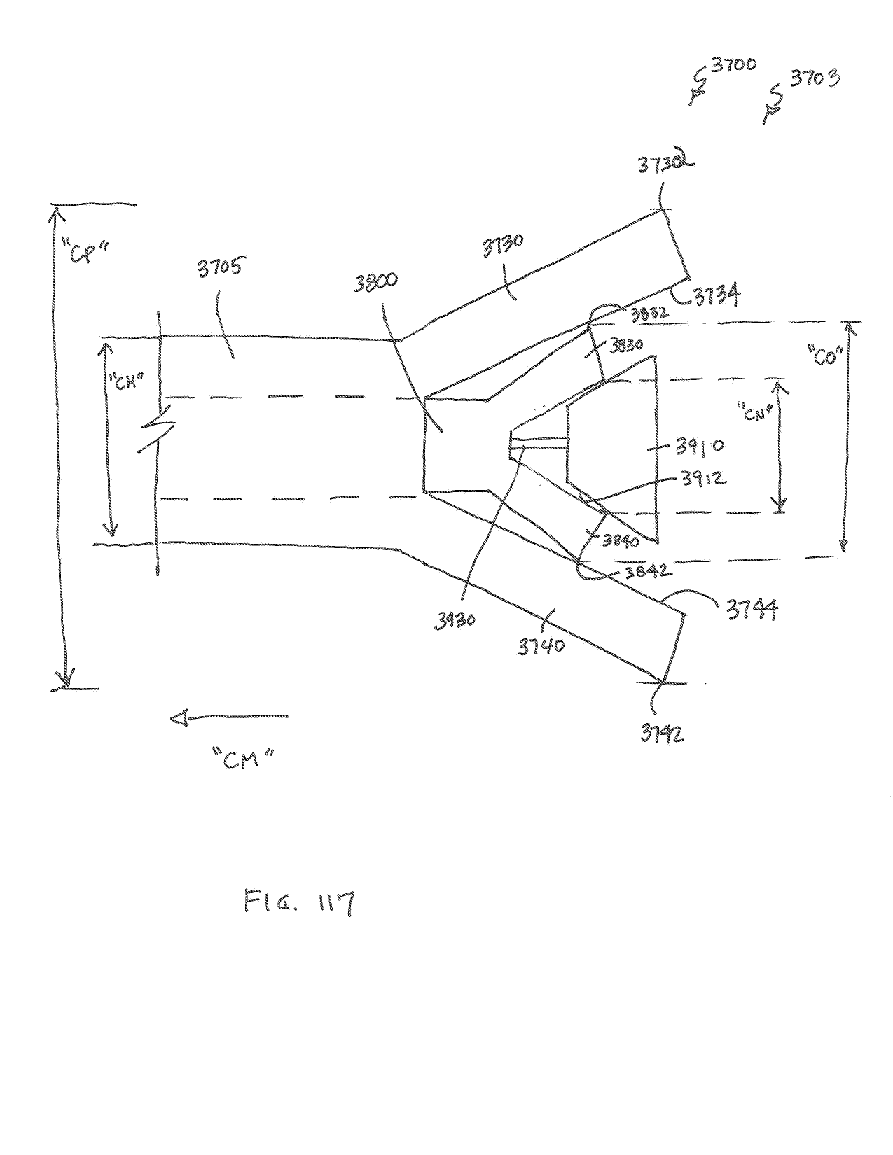

Referring to FIGS. 113 and 114, a side view and an end view of an embodiment of an expander portion in accordance with the invention are illustrated, respectively. In this embodiment, the expander portion 3800 includes a body 3810 with an outer surface 3812 that has an outer diameter of “CJ.” The expander portion 3800 is illustrated in its delivery configuration 3850 in FIG. 113. As the expander portion 3800 is movable relative to the cutting tool 3705, the outer diameter “CJ” is slightly less than the inner diameter “CI” of the channel 3714 of the cutting tool 3705. The body 3810 includes a channel 3814 that is defined by an inner surface 3816 with an inner diameter “CK.” The channel 3814 extends toward the distal end 3818 of the expander portion 3800.

The expander portion 3800 includes expanding elements 3830 and 3840 that are separated by notches 3820 and 3824 that are formed in the body 3810. The notches 3820 and 3824 are formed by the removal of material and extend to surfaces 3822 and 3826, respectively.

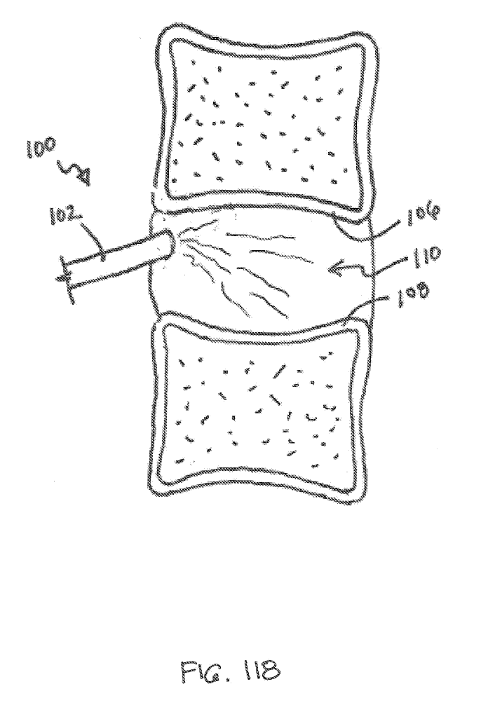

Referring to FIGS. 115 and 116, a side view and an end view of an embodiment of an expander portion in accordance with the invention are illustrated, respectively. In this embodiment, the expander portion 3900 includes a body 3910 that has a tapered or angled surface 3912 that extends from end 3914 to end 3916. The surface 3912 is a deflecting or expanding surface and while the surface 3912 is illustrated as being substantially linear or smooth, the surface in other embodiments can have a curved or otherwise non-linear shape or configuration. The extent to which the surface 3912 is tapered or angled relative to a longitudinal axis, such as axis 3918, is determined by the difference between the outer diameter of surface 3914 and the outer diameter of surface 3916. As the outer diameter of surface 3916 increases relative to the outer diameter of surface 3914, the angle of surface 3912 relative to axis 3918 increases, thereby resulting in a quicker or more dramatic expansion of the cutting elements 3730 and 3740.

The expander portion 3900 includes an actuator 3930 that is coupled to the body 3910. In one embodiment, the actuator 3930 can be a separate, elongate member that is coupled at end 3932 to the body 3910. The actuator 3930 is configured to extend through the channel 3814 of expander portion 3800 to the proximal end of the site preparation tool 3705 so that a user can pull or move the actuator 3930 proximally to move the body 3910 relative to the expander portion 3800. In other embodiments, the actuator 3930 can be integrally formed with the body 3910.

Referring to FIG. 117, the site preparation tool 3700 is illustrated in a deployed configuration 3703. As shown, the cutting elements 3730 and 3740 can be expanded outwardly by the expanding mechanism. Initially, when the expansion of the cutting tool 3705 and in particular, of the cutting elements 3730 and 3740, is desired, the user can move the actuator 3930 proximally along the direction of arrow “CM.” Movement of the actuator 3930 along that direction will cause the body 3910 to move along the same direction. As the body 3910 moves along the direction of arrow “CM,” the engaging elements 3830 and 3840 are contacted and engaged by the deflecting surface 3912 on the body 3910. As the body 3910 continues to move along the direction of arrow “CM,” the engaging elements 3830 and 3840 of the expander portion 3800 are moved increasingly outwardly.

In FIG. 117, the expanding elements 3830 and 3840 contact the surface 3912 of body 3910 at points that define a distance “CN” therebetween. At the same time, the outer tips or edges 3832 and 3842 of the expanding elements 3830 and 3840 define a distance “CO.” The tips or edges 3832 and 3842 engage the inner surfaces 3734 and 3744 of the cutting elements 3730 and 3740, respectively. As a result, the cutting elements 3730 and 3740 expand from the outer diameter “CH” in the delivery configuration 3702 to an outer diameter “CP” in the deployed configuration 3703 as defined by tips or edges 3732 and 3742. The two-stage expanding mechanism enables the cutting elements 3730 and 3740 to be expanded wider than a single-stage expanding mechanism as the outer diameters of the components of the site preparation tool 3700 are limited by, and cannot be greater than, the inner diameter of the delivery device.

Thus, referring to FIG. 117, the outer diameter “CH” of the cutting tool 3705 is slightly less than the inner diameter of a delivery device. As the expanding elements 3830 and 3840 are expanded at least to the distance “CO,” which is wider than the outer diameter “CH,” the cutting elements 3730 and 3830 can be expanded wider to a greater degree than if a single stage expanding mechanism is used.

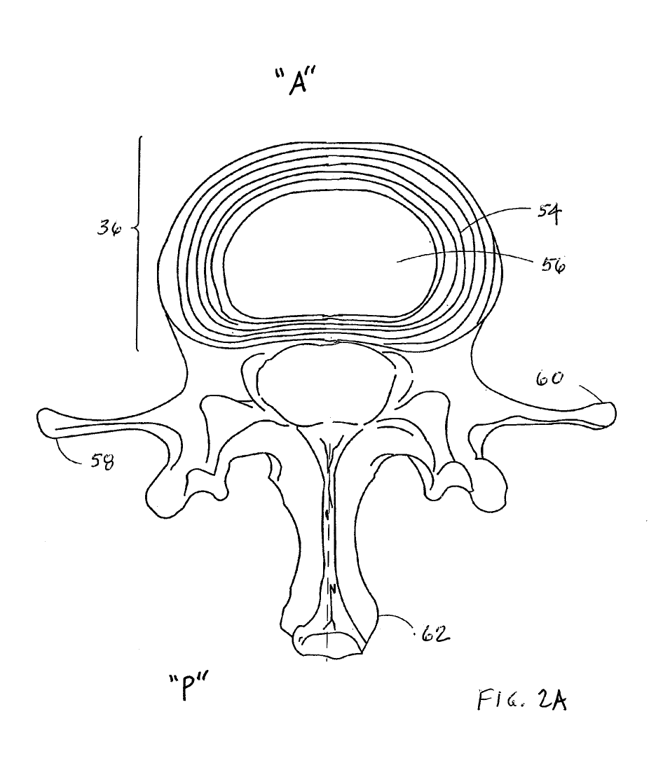

As shown in FIGS. 2-4, a method for performing a percutaneous spine procedure includes a physician-user inserting delivery device 102 and preparation or engaging device 104 into disc space 110 or facet joint 74 through a skin exit location (not shown). As discussed previously herein, delivery device 102 can be a needle, cannula or other tube-like structure that has an internal channel through which preparation or engaging device 104 can be inserted. Please note that the terms “engaging device” and “preparation device” are used interchangeably herein to mean an end plate or facet joint trauma device. Delivery device 102, as well as the other delivery devices described herein, has an outer diameter dimension (see “OD” in FIG. 3) that will range between 0.5 and 5 millimeters with a more detailed range of 1.3 to 3.5 millimeters. As discussed below, the OD of delivery device 102 will generally be determined based on the type and location of the approach to insert delivery device 102. Following the insertion of delivery device 102 into the patient's body through the skin, a percutaneous pathway is established by moving delivery device 102 inwardly until distal end 103 is located within disc space 110 as defined in part by endplates 106 and 108 or facet joint 74 as defined by the inferior facet 77 and superior facet 76. Although not shown, it should be understood to those skilled in the art that delivery device 102 may be used in association with a stylet to ensure post-insertion patency of the cannula.

Insertion of delivery device 102 into disc space 110 may be performed under fluoroscopic guidance using at least two acceptable anatomic approaches. Such approaches may be conducted either unilaterally or bilaterally, depending upon the anatomic restrictions of the patient. The first approach is a standard extrapedicular discographic approach and the second approach is a more lateral approach, in which delivery device 102 is introduced from a more “sideways” angle. The extrapedicular discographic approach will generally use a smaller gauge of instrumentation (i.e. 14- or 16-gauge) than the lateral approach (8-, 10- or 12-gauge). It should be understood to an artisan skilled in the art that the size determination of delivery device 102 will be determined by the physician-user depending upon the presented clinical condition.

Insertion of delivery device 102 into facet joint 74 (not shown together) may be performed under fluoroscopic guidance using a posterior approach. The approaches may be conducted either unilaterally or bilaterally, depending upon whether one or both facet joints of the motion segment are to be treated. Preparation of the facet joint will generally use a smaller gauge of instrumentation (i.e. 20- or 18- or 16-gauge) than then treatment of the disc space. It should be understood to an artisan skilled in the art that the size determination of delivery device 102 will be determined by the physician-user depending upon the presented clinical condition.

The method may include the physician-user confirming proper delivery device 102 placement in the posterior-lateral disc annulus by obtaining anterior-posterior and lateral fluoroscopic views. After the position is confirmed, if used the stylet may be removed from delivery device 102. Engaging device 104 or preparation device is subsequently inserted through delivery device 102 into the mid-portion of a disc (not shown). To ensure functionality, engaging device 104 must generally fit within delivery device 102 and be capable of some order of decortication/tissue trauma within disc space 110. Engaging device 104 may retain its pre-insertion geometry once deployed, or may assume a different geometry upon deployment. If there is a geometric change, it may be due to the physical nature of the device (e.g. made of shape-memory material) or to triggering by the physician-user. Several embodiments of engaging tool 104 have been described previously herein that address these described functional requirements, thus for brevity sake these associated structural features will not be described again here.

The method may alternatively or additionally include the physician-user confirming proper delivery device 102 placement in the facet joint 74 by obtaining anterior-posterior and lateral fluoroscopic views. After the position is confirmed, if used, the stylet may be removed from the delivery device 102. Engaging device 104 or preparation device is subsequently inserted through the delivery device 102 into the mid-portion of a facet (not shown). To ensure functionality, engaging device 104 must generally fit within delivery device 102 and be capable of some order of decortication/tissue trauma within facet joint 74. Engaging device 104 may retain its pre-insertion geometry once deployed, or may assume a different geometry upon deployment. If there is a geometric change, it may be due to the physical nature of the device (e.g. made of shape-memory material) or to triggering by the physician-user. Several embodiments of engaging tool 104 have been described previously herein that address these described functional requirements, thus for brevity sake, these associated structural features will not be described again here.

As seen in FIG. 4, the method provides further that following the insertion of engaging device 104 through delivery device 102, engaging device 104 can be moved by the physician-user repeatedly along the directions of arrows “A” and “B” to engage the target area, which in the example illustrated in FIG. 3 is end plate 106. Physician-user may bluntly dissect disc space 110 to establish the anterior border of the disc with this position being marked on engaging device 104. In a systematic manner, engaging device 104 is moved back and forth or rotated within disc space 110 or facet joint 74 to mechanically debride or abrade both superior endplate 106 and inferior end plate 108. Engaging device 104 may be moved longitudinal (in-out), axially rotated, or some combination thereof. It should be understood to one skilled in that art that during this step, it may be necessary to re-angle delivery device 102 to achieve maximum debridement. The level of abradement or debridement/decortication (depending on the level of disc degeneration) is judged on the aspiration of blood through delivery device 102. As further illustrated in FIG. 4, engaging device 104 may be used to scrap or break the endplate 106 in area 112 and cause the flow of blood 116 into disc space 110. In addition or alternatively, the engaging device can be used to scrape the articulating surfaces of facet joint 74 and cause the flow of blood into the facet joint. Engagement device 104 can be used to penetrate either end plate 106, 108 as well. To induce the flow of blood, while it is not required that endplate 106, 108 be broken through to the cancellous portion 114, that would be the easiest manner in which to achieve blood flow. In one exemplary method, the physician-user may withdraw engaging device 104 through delivery device 102 and inspect engaging device 104 for the presence of blood. If no blood is present on engaging device 104, the physician-user may re-insert engaging device 104 and repeat the cutting or scraping process. When the process is complete, engaging device 104 is withdrawn along the direction of arrow “C” and removed from the patient.

The method also optionally includes aspirating blood, and any generated bone debris and/or disc material through the cannula of delivery device 102 following the determination by the physician-user that the level of trauma or abradement inflicted onto superior endplate 106 and inferior end plate 108 is considered sufficient to induce the flow of blood 116 into disc space 110.

The method may further include the delivery of a biomaterial into the prepared disc space 110 or facet joint 74 to facilitate the formation of a bone fusion or alternatively, a partial arthrodesis between two adjoining vertebrae or between the facet joint articulating surfaces. As shown in FIG. 110, the biomaterial may be injected into disc space 110 using delivery device 102. The biomaterial may be similarly injected into the facet joint 74. The biomaterial is generally a non-curable, biocompatible, material that includes in its composition, a biologic agent. It is contemplated that the biomaterial may be a gel-like substance, or alternatively, the biomaterial may also have a paste-like consistency. This biologic agent may be chosen from a group of agents including, but not limited to, methylcellulose, carboxymethlycellulose, tri-calcium phosphate, calcium sulfate, hyaluranic acid, sodium hyaluranate, bio-active glass, collagen, hydroxyl appetite, calcium salts, fibrin, diglycidyl polyethyleneglycol, chitin derivatives including chitosan, polyvinylpyrrolidone (PVP), polycaprolactone (PCL), carboxymethycellulose and other cellulose derivatives. The biomaterial may also have in its composition a component for inducing bone growth and facilitating forming a biological fusion, or alternatively a partial arthrodesis between two adjacent endplates or facet joint articulating surfaces. Components contemplated for inducing bone growth or fusion generation may include a bone morphogenic protein (BMP), demineralized bone matrix (DBM), or growth factors. Further yet, the biomaterial may undergo a cell seeding procedure before its delivering into disc space 110 to increase its osteoinductive, osteoconductive, and/or osteogenic behavior in vivo.

The biomaterial may also include in its composition a contrast component that allows the physician-user to visualize the material during the delivery process to disc space 110 or facet joint 74 under direct fluoroscopy. This would allow the physician-user to determine whether the biomaterial is being placed in the correct location or whether sufficient volume of the biomaterial has been delivered within disc space 110 or facet joint 74. Generally, the biomaterial will be delivered in a single dose through delivery device 102. In the event insufficient biomaterial has been injected, subsequent additional dosages may be provided through delivery device 102.

The method may further include the delivery of autologous or allograft materials into the prepared disc space 110 or facet joint 74 to facilitate the formation of a bone fusion or alternatively, a partial arthrodesis between two adjoining vertebrae or between the facet joint articulating surfaces. As shown in FIG. 110, the materials may be injected into disc space 110 using delivery device 102. The materials may be similarly injected into the facet joint 74. The autologous materials include, but are not limited to bone graft, bone marrow aspirate, concentrated or unconcentrated blood products or platelet rich plasma. The autologous materials may be obtained from the patient via open surgical, needle based aspiration or blood drawing and concentrating techniques from the patient at the time of the spine procedure or during a prior procedure. It is contemplated that the autologous materials may delivered without modification or may be concentrated or combined with the biomaterials listed above or other agents to adjust consistency or improve the biologic response

The method may also include withdrawing delivery device 102 from disc space 110 or facet joint 74 and removing it through the skin exit location (not shown). A removable sterile bandage is usually placed over the skin exit location wound to prevent infection.

Post-procedure, the method provides for the patient to wear a temporary external back brace, spine isolation device or support mechanism sized for the levels that may be impacted by the percutaneous spine procedure for a time prescribed by the physician-user. The external support mechanism is configured to substantially restrict motion at a certain spine level to, thereby allow bone growth, fusion or an arthrodesis to form.

In various embodiments, the materials and configurations of the components can vary depending on the properties and functionality desired for the particular component.

While the invention has been described in detail and with references to specific embodiments thereof, it will be apparent to one skilled in the art that various changes and modifications can be made therein without departing from the spirit and scope thereof. Thus, it is intended that the present invention covers the modifications and variations of this invention provided they come within the scope of the appended claims and their equivalents.

What is claimed is:

1. A tool for causing blood flow into a disc space, the disc space being defined in part by at least one vertebral endplate, the tool comprising:
a first cutting element, the first cutting element being selectively disposable in a delivery configuration and in a deployed configuration, the first cutting element including a free end and being configured to engage the at least one vertebral endplate;
a second cutting element, the second cutting element being selectively disposable in a delivery configuration and in a deployed configuration, the second cutting element including a free end and being configured to engage the at least one vertebral endplate; and
a deflecting element, the deflecting element including a body portion having a deflecting surface, the deflecting surface being configured to direct the first cutting element and the second cutting element outwardly as each of the first cutting element and the second cutting element engages the deflecting surface.

2. The tool of claim 1, wherein the deflecting element is disposed between the first cutting element and the second cutting element.

3. The tool of claim 1, wherein the first cutting element has a distal end, the second cutting element has a distal end, and a portion of the deflecting element extends beyond the distal ends of the first and second cutting elements when the first and second cutting elements are in their delivery configurations in which they are moved to the disc space.

4. The tool of claim 1, wherein the first cutting element, the second cutting element, and the deflecting element are configured to be moved substantially simultaneously along a delivery device to the disc space.

5. The tool of claim 1, wherein the deflecting element is configured to move relative to and engage the first cutting element and the second cutting element so that each of the first cutting element and the second cutting element moves from its delivery configuration to its deployed configuration in which it is spaced apart from the other cutting element.

6. The tool of claim 1, wherein the first cutting element and the second cutting element collectively define an outer diameter within the range of 0.8 mm to 3.4 mm when the first cutting element and the second cutting element are in their delivery configurations.

7. The tool of claim 1, wherein the first cutting element and the second cutting element are configured to be moved along a delivery device to the disc space, the delivery device having an outer diameter, and the first cutting element and the second cutting element collectively define an outer diameter within the relative range of 1.0 times the outer diameter of the delivery device to 3.0 times the outer diameter of the delivery device when the first cutting element and the second cutting element are in their deployed configurations.

8. The tool of claim 1, wherein the first cutting element, the second cutting element, and the deflecting element can be rotated at the same time.

9. A system for inducing blood flow into a disc space, the disc space being defined in part by a vertebral endplate, the system comprising:
a cutting mechanism, the cutting mechanism being configured to engage the vertebral endplate, the cutting mechanism being selectively disposable in a delivery configuration in which the cutting mechanism has a first profile and in a deployed configuration in which the cutting mechanism has a second profile, the second profile being larger than the first profile, the cutting mechanism including at least one free end;
an expander mechanism, the expander mechanism being configured to engage the at least one free end of the cutting mechanism to move the cutting mechanism from its delivery configuration to its deployed configuration; and
a control mechanism, the control mechanism being coupled to the cutting mechanism, the control mechanism being configured to be used to impart motion to the cutting mechanism in its deployed configuration in which the at least one free end engages the vertebral endplate.

10. The system of claim 9, wherein the cutting mechanism includes a first cutting component and a second cutting component, and the expander mechanism is disposed between the first cutting component and the second cutting component.

11. The system of claim 10, wherein each of the first cutting component and the second cutting component is selectively disposable in a delivery configuration and in a deployed configuration and is configured to engage the vertebral endplate, the first cutting component contacting the second cutting component in its delivery configuration and being spaced away from the second cutting component in its deployed configuration.

12. The system of claim 9, wherein the cutting mechanism has a distal end and the expander mechanism has a distal end, the distal end of the expander mechanism being disposed beyond the distal end of the cutting mechanism when the cutting mechanism is in its delivery configuration.

13. The system of claim 9, wherein the expander mechanism includes an actuator, and engagement of the actuator with the at least one free end causes the cutting mechanism to move to its deployed configuration in which the at least one free end extends radially outwardly.

14. The system of claim 9, wherein the control mechanism is configured to rotate the cutting mechanism.

15. The system of claim 9, wherein the cutting mechanism includes a first cutting element and a second cutting element, the first cutting element including a free end and the second cutting element including a free end, the expander mechanism being configured to engage the first cutting element and the second cutting element to cause the free ends of the cutting elements to move outwardly, the free ends being configured to engage the vertebral endplate.

16. A site preparation device for inducing blood flow into a disc space, the disc space being defined in part by at least one vertebral endplate, the site preparation device comprising:
a delivery device, the delivery device including an elongate body having a proximal end, a distal end, and a channel extending from the proximal end to the distal end, the body including a longitudinal axis; and
a tool, the tool being configured to be inserted through the channel of the body, the tool including:
a cutting element, the cutting element including a proximal end and a distal end, the distal end being a free end, the cutting element being selectively disposable in a delivery position substantially aligned with the longitudinal axis of the shaft and in a deployed position extending away from the longitudinal axis of the shaft, the cutting element being configured to engage the at least one vertebral endplate and cause blood to flow into the disc space; and
an actuating element, the actuating element being movable to engage the cutting element to move the cutting element from its delivery position to its deployed position.

17. The site preparation device of claim 16, wherein the cutting element is a first cutting element, and the tool includes a second cutting element having its own proximal end and distal end, the distal end of the second cutting element being a free end, the actuating element being configured to engage the second cutting element to move the second cutting element from a delivery position to a deployed position.

18. The site preparation device of claim 17, wherein the actuating element is configured to engage the free end of the first cutting element and the free end of the second cutting element substantially simultaneously.

19. The site preparation device of claim 17, wherein the channel of the delivery device defines an inner diameter, the first cutting element and the second cutting element are disposable proximate to each other in their delivery positions and collectively defining an outer diameter in those positions, the outer diameter being substantially the same as the inner diameter of the channel.

20. The site preparation device of claim 19, wherein the actuating element includes a support portion and an actuator portion, the support portion being configured to be disposed between the first cutting element and the second cutting element when the first cutting element and the second cutting element are in their delivery positions and disposed in the channel of the delivery device.

21. The site preparation device of claim 20, wherein the actuator portion is configured so that it has an outer dimension substantially the same as the inner diameter of the channel and the outer diameter defined by the first cutting element and the second cutting element.

22. A method of performing a percutaneous spine procedure on a patient, the method comprising:
inserting a delivery device into a spinal column of a patient;
establishing a percutaneous pathway using the delivery device leading from a skin exit location to a disc space defined by at least one vertebral endplate;
introducing a preparation device through the delivery device to the disc space, the preparation device including a support portion and a cutting portion, the cutting portion of the preparation device being selectively disposable in a delivery configuration and in a deployed configuration relative to the support portion;
preparing the disc space by engaging the cutting portion of the preparation device with at least one vertebral endplate; and
delivering a biomaterial through the delivery device to the prepared disc space to facilitate forming at least a partial arthrodesis between two adjacent endplates.

23. The method of claim 22, wherein the biomaterial comprises a biocompatible, gel-like material that conforms to a geometry and maintains a defined shape after delivery to facilitate forming at least a partial arthrodesis between two adjacent endplates.

24. The method of claim 22, where the biocompatible, gel-like material includes a contrast material.

25. The method of claim 23, where the biocompatible, gel-like material is a non-curable material.

26. The method of claim 23, wherein the biocompatible, gel-like material includes a biologic agent.

27. The method of claim 26, wherein the biologic agent comprises at least one of methylcellulose, carboxymethlycellulose, tri-calcium phosphate, calcium sulfate, hyaluranic acid, sodium hyaluranate, bio-active glass, collagen, calcium salts, hydroxyl appetite, diglycidyl polyethyleneglycol, chitin derivatives including chitosan polyvinylpyrrolidone (PVP), polycaprolactone (PCL), carboxymethycellulose and other cellulose derivatives.

28. The method of claim 22, wherein the biomaterial includes material for inducing bone growth and facilitating forming at least a partial arthrodesis between two adjacent endplates.

29. The method of claim 28, wherein the material for inducing bone growth is chosen from bone morphogenic protein (BMP), demineralized bone matrix (DBM), and growth factors.

30. The method of claim 22, further comprising seeding the biomaterial with cells before delivering the biomaterial through the delivery device to the prepared disc space.

31. The method of claim 22, wherein the preparing further comprises preparing the disc space by damaging the at least one vertebral endplate to create a bleeding fusion bed to receive the biomaterial.

32. The method of claim 22, wherein the inserting further comprises inserting the delivery device using at least one of an extrapedicular discographic approach or a lateral approach to access the spinal column of a patient.

33. The method of claim 32, which the extrapedicular discographic approach may be at least one of unilateral or bilateral relative to the spinal column.

34. The method of claim 32, wherein the lateral approach may be at least one of unilateral or bilateral relative to the spinal column.
